(19) United States
(12) Patent Application Publication
(10) Pub. No.: US 2015/0258039 A1
(43) Pub. Date: Sep. 17, 2015

(54) PREPARATION OF GGA AND DERIVATIVES THEREOF AND THEIR CO-CRYSTALLIZATION WITH UREA OR THIOUREA

(71) Applicant: COYOTE PHARMACEUTICALS, INC., Menlo Park, CA (US)

(72) Inventors: Abril-Hörpel Obsidiana, Menlo Park, CA (US); William Haag, Menlo Park, CA (US); Ann Pierson, Menlo Park, CA (US)

(73) Assignee: COYOTE PHARMACEUTICALS, INC., Menlo Park, CA (US)

(21) Appl. No.: 14/433,018

(22) PCT Filed: Sep. 30, 2013
(86) PCT No.: PCT/US2013/062708

§ 371 Date: Apr. 1, 2015

Related U.S. Application Data
(63) Continuation of application No. 13/815,805, filed on Mar. 15, 2013, now abandoned.
(60) Provisional application No. 61/708,570, filed on Oct. 1, 2012.

(51) Int. Cl. A61K 31/045; C07C 45/29; C07C 45/42; A61K 47/18; A61K 47/20

(52) U.S. Cl. CPC A61K 31/045; A61K 47/18; A61K 47/20; C07C 45/42; C07C 45/29; C07B 2200/13; C07B 2200/09

(57) ABSTRACT

Provided herein are compositions comprising co-crystals of GGA or a GGA derivative (including salts and tauotomers thereof) with urea or thiourea, and processes related to such co-crystals. Also provided herein are methods, compounds, and compositions related to preparing trans GGA and derivatives thereof, and intermediates thereto.

### FIELD OF THE INVENTION

This invention provides compositions comprising co-crystals or co-precipitates of geranylgeranylacetone (GGA) or a GGA derivative (including salts and tauotomers thereof) with urea or thiourea. Preferably, such co-crystals or co-precipitates are enriched in the all trans isomer of the GGA or the GGA derivative, compared to the relative amount of the trans isomer in the mixtures of cis and trans isomers of GGA or the GGA derivatives employed to prepare such co-crystals. This invention also relates to facile processes for isolating substantially pure cis and trans, preferably the trans, GGA and GGA derivatives from the mixture of the corresponding cis and trans isomers. Furthermore, this invention relates to preparing trans GGA and derivatives thereof, and intermediates thereto.

### STATE OF THE ART

GGA has the formula:

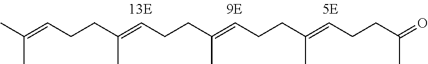 [SMILES: CC(=O)CC/C=C(\C)CC/C=C(\C)CC/C=C(\C)CCC=C(C)C]

and is reported to have neuroprotective and related effects. See, for example, PCT Pat. App. Pub. Nos. WO 2012/031028, WO/2013/052148, WO/2013/130648, WO/2013/130242, and WO/2013/130654, each of which is incorporated herein by reference in its entirety. All trans (or 5E, 9E, 13E) GGA is, surprisingly, more effective as a neuroprotective agent compared to 5-cis (or 5Z, 9E, 13E) GGA. However, conventional synthesis of GGA or GGA derivatives can provide a mixture of cis and trans isomers of GGA or GGA derivatives, such as the 5E and the 5Z isomers, which can be difficult, if not impossible to separate, especially in a scale amenable to large scale manufacturing. Furthermore, the process of separating the isomers and the compounds or compositions should be robust and reproducible enough to provide compounds and compositions repeatedly with the same or substantially the same specification as required in the regulated pharmaceutical industry.

### SUMMARY OF THE INVENTION

In some aspects, this invention provides compositions comprising co-crystals or co-precipitates of GGA or a GGA derivative (including salts and tauotomers thereof) with urea and/or thiourea, and processes related to such co-crystals. Preferably, the co-crystals include substantially more of the all-trans (hereinafter “trans”) form or substantially only the trans form of the GGA or the GGA derivative. As used herein, “substantially” in the context of cis/trans configurations refers to at least 80%, more preferably at least 90%, yet more preferably at least 95%, and most preferably at least 99% of the desired configuration. The co-crystals can include at least 80%, more preferably at least 90%, yet more preferably at least 95%, and most preferably at least 99% of the trans isomer.

Without being bound by theory, urea and thiourea form channels in the solid state which channels are shaped to preferably accommodate a trans isomer of GGA or a GGA derivative over a corresponding cis isomer. Thus, when a mixture of trans and cis isomers of GGA or a GGA derivative is co-crystallized with urea or thiourea, the trans isomers of GGA and GGA derivatives can be selectively complexed and/or isomerized from a cis isomer within these channels, and provide a higher relative amount of the trans isomer (over the corresponding cis isomer) in the co-crystal compared to the relative amount of the trans isomer in the starting mixture.

In a related aspect, this invention also provides, at least 80%, more preferably, at least 90%, yet more preferably, at least 95%, and most preferably, at least 99% of the trans isomer of GGA or a GGA derivative.

In certain important composition aspects, this invention relates to co-crystals of GGA and the following GGA derivatives, which are GGA intermediates:

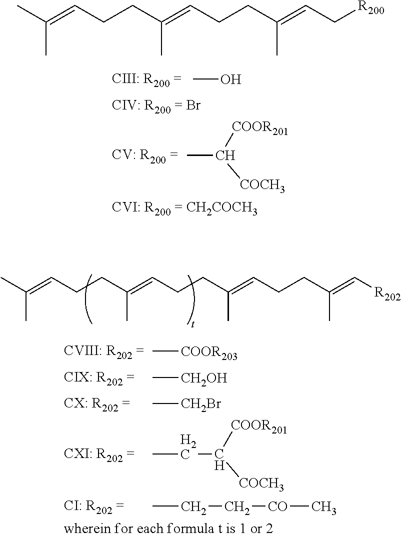

with urea or thiourea. When the bromide is employed, urea can be the preferred crystallizing agent. In certain embodiments where the co-crystal contains GGA, at least 90%, more preferably, at least 95%, yet more preferably at least 99%, and most preferably, at least 99.5% of the GGA or the GGA derivative is present as a trans isomer. Within these aspects, in a preferred embodiment, the co-crystal includes thiourea as the channel forming agent.

In a composition aspect, provided herein is a co-crystal or co-precipitate comprising

GGA or a GGA derivative, and

urea and/or thiourea,

wherein the GGA or the GGA derivative exists at least 80%, or at least 90%, or at least 95%, or at least 99% in the trans isomer. In one embodiment, provided herein is the co-crystal or co-precipitate of this invention admixed with a composition comprising GGA or the GGA derivative, wherein the GGA or the GGA derivative in the composition exists substantially less in the trans form compared to the GGA or the GGA derivative that is complexed as part of the co-crystal or the co-precipitate. In another embodiment, the co-crystal or co-precipitate provided herein is crystalline.

In another aspect, provided herein is a crystalline GGA derivative existing in the trans form or substantially in the trans form.

In another aspect, provided herein are cis-trans mixtures of GGA and GGA derivative existing substantially in the cis form.

This invention also provides processes for preparing co-crystals of GGA or GGA derivatives, and for preparing GGA or a GGA derivative containing at least 80%, more preferably, at least 90%, yet more preferably, at least 95%, and most preferably, at least 99% of the trans isomer of GGA or a GGA derivative.

In certain important process embodiments, this invention provides processes for preparing co-crystals of and the following GGA derivatives:

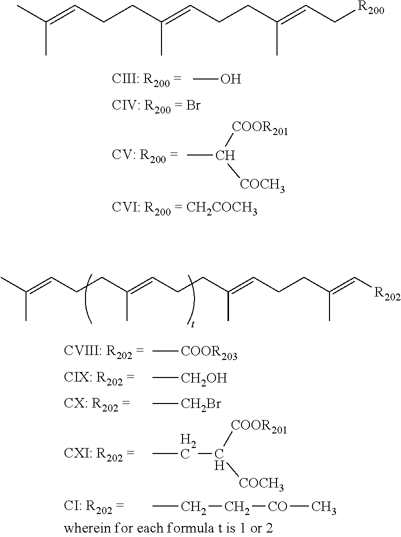

with urea or thiourea, comprising contacting GGA or the above mentioned GGA derivative with urea or thiourea to provide co-crystals of GGA or the GGA derivative with urea or thiourea. In certain embodiments, such co-crystals contain at least 90%, more preferably, at least 95%, yet more preferably at least 99%, and most preferably, at least 99.5% of the GGA or the GGA derivative as a trans isomer.

In certain preferred embodiments, the contacting is performed in a solvent. In certain other embodiments, for example, when the GGA or the GGA derivative exists in a liquid state, such a liquid, containing substantially no solvent, can be contacted with solid urea or thiourea. The GGA or the GGA derivative, and the urea or thiourea is contacted preferably in a molar ratio of about 1:1. An excess, such as 50, 100, or 150 mole % of the urea or the thiourea can be used to drive the co-crystal formation. A less than 1 molar equivalent of the urea or the thiourea can also be used. The co-crystal formation can be monitored by analyzing the cis/trans content of the reaction liquid, e.g., by HPLC. Other methods for complexing other compounds are well known in the art and can be adapted by the skilled artisan in view of this disclosure to prepare the co-crystals of this invention.

In another process aspect, this invention provides processes for preparing GGA or a GGA derivative, wherein at least 80%, more preferably, at least 90%, yet more preferably at least 95%, and most preferably, at least 99% of the GGA or the GGA derivative is present as a trans isomer, the processes comprise isolating from a co-crystal of GGA or a GGA derivative with urea or thiourea, wherein at least 80%, more preferably, at least 90%, yet more preferably at least 95%, and most preferably, at least 99% of the GGA is present as a trans isomer, the GGA or the GGA derivative.

In certain important process embodiments, this invention provides processes for preparing GGA the following GGA derivatives:

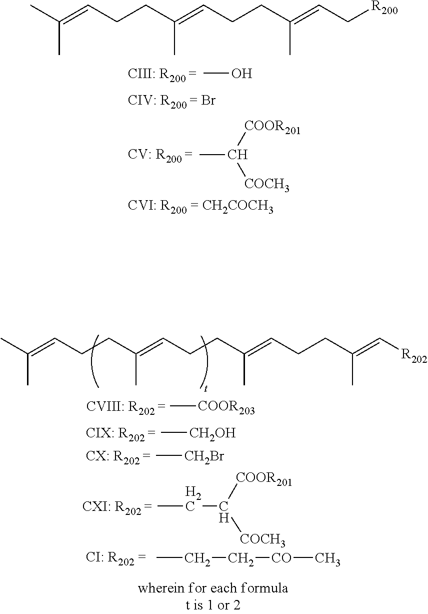

wherein at least 90%, more preferably, at least 95%, yet more preferably at least 99%, and most preferably, at least 99.5% of the GGA or the GGA derivative is present as a trans isomer, the processes comprising isolating from a co-crystal of GGA or the GGA derivative with urea or thiourea wherein at least 90%, more preferably, at least 95%, yet more preferably at least 99%, and most preferably, at least 99.5% of the GGA or the GGA derivative is present as a trans isomer, the GGA or the GGA derivative. In any of the embodiments described, each R₂₀₁ and R₂₀₃ is independently hydrogen or C₁-C₆ alkyl.

As provided herein above, in a preferred embodiment, t is 1. As provided herein above, in another preferred embodiment, t is 2.

For the isolating, the co-crystal can be decomposed with a solvent such as water, and extracted with a solvent in which the GGA is soluble but urea or thiourea is not soluble. Alternatively, for a solid GGA derivative, its co-crystal can be decomposed with a solvent in which the derivative is insoluble or substantially insoluble, such as methanol, and the precipitated solid GGA derivative isolated by filtration. Other isolation methods well known in the art can be adapted by the skilled artisan in view of this disclosure to isolate GGA and GGA derivatives from their corresponding co-crystals as provided herein.

In a process aspect, provided herein is a process of preparing a co-crystal or a co-precipitate of GGA or a GGA derivative, and urea and/or thiourea, wherein the GGA or the GGA derivative exists at least 80%, or at least 90%, or at least 95%, or at least 99% as the trans isomer, the process comprising contacting a mixture of cis and trans isomers of GGA or the GGA derivative, in which mixture the percentage of the trans isomer is lower than that in the complexed co-crystal, with a composition comprising urea and/or thiourea under conditions sufficient to form the co-crystal or the co-precipitate, to provide the co-crystal or the co-precipitate. In one embodiment, the process further comprises isolating the GGA or the GGA derivative from the co-crystal, to provide the GGA or the GGA derivative having at least 80%, or at least 90%, or at least 95%, or at least 99% of the trans isomer.

In another aspect, provided herein is a process of separating GGA or a GGA derivative existing in the trans form or substantially in the trans form, the process comprising contacting a co-crystal or co-precipitate comprising GGA or the GGA derivative, and urea and/or thiourea, wherein the GGA or the GGA derivative complexed in the co-crystal or the co-precipitate exists in the trans form or substantially in the trans form, with a solvent that selectively dissolves either the GGA or the GGA derivative, or the urea and/or the thiourea, under conditions sufficient for such dissolution. The purity of the GGA or the GGA derivative thus dissolved and isolated can be determined by various methods, including by HPLC. The isolated GGA or the GGA derivative can be further purified by washing with a solvent that selectively dissolves urea or thiourea over GGA or the GGA derivative, or extracting with a solvent that dissolves the GGA or the GGA derivative selectively over urea or thiourea.

While the disclosure refers to urea and thiourea in particular, it will be apparent to the skilled artisan that alkylated and other derivatives of urea and thiourea, that are known or are capable of forming channels similar to urea and thiourea, are also contemplated to be useful in this invention.

In various method aspects as provided herein, and preferably herein above, GGA or GGA derivative is obtained substantially in a cis form comprising separating the co-crystal or co-precipitate from the corresponding mother liquor, and obtaining the GGA or the GGA derivative substantially in the cis form in the mother liquor. The solvents can be removed from the mother liquor to provide the GGA or the GGA derivative substantially in the cis form.

In some preferred embodiments, the GGA, geranylgeranyl alcohol, or the another GGA derivative utilized herein exist less than 50%, less than 80%, less than 90%, or less than 95% as the cis isomer. In some preferred embodiments, the GGA, geranylgeranyl alcohol, or the another GGA derivative utilized herein exist less than 50%, less than 80%, less than 90%, or less than 95% as the trans isomer.

In various aspects, provided herein are processes of syntheses of GGA and derivatives thereof, and intermediates thereto.

In one aspect, a process for preparing a compound of formula (XXXI) is provided,

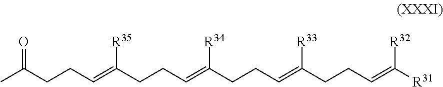

said process comprising:
hydrolyzing a compound of formula (XXXII):

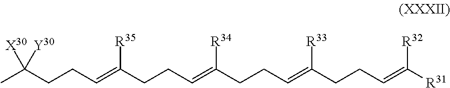

wherein:
X³⁰ and Y³⁰ are each independently OR³⁶, SR³⁶, or X³⁰ and Y³⁰ together with the carbon atom they are attached to form a ring of formula:

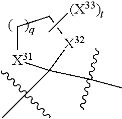

wherein each R³⁶ is independently C₁-C₆ alkyl,
each X³¹ and X³² are independently O, or S; q is 1 or 2; each X³³ is independently C₁-C₆ alkyl; t is 0, 1, 2, or 3, and
each of R³¹, R³², R³³, R³⁴, and R³⁵ is independently H or C₁-C₆ alkyl or R³¹ and R³² together with the carbon atom they are joined to form a C₅-C₆ cycloalkyl optionally substituted with 1-3 C₁-C₆ alkyl.

In further aspects, a process for preparing a compound of formula (XXXII) is provided:

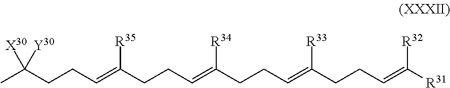

said process comprising:
contacting a compound of formula (XXXIII):

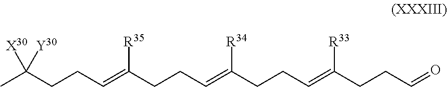

wherein:
the variables are defined as in formula (XXXII) above
with a compound of formula:

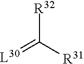

wherein L³⁰ is P(R^{z})₃, P(O)(R^{z})₂, SO₂R^{z}, Si(R^{z})₃, preferably P(R^{z})₃; and wherein is R^{z} is a C₁-C₆ alkyl group or an aryl group;
under conditions suitable for olefination of compound of formula (XXXIII) to produce a compound of formula (XXXII).

In further aspects, a process for preparing a compound of formula (YXI) is provided:

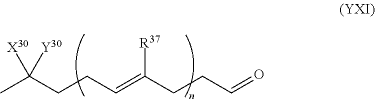

wherein R³⁷ is defined as R³³, R³⁴ or R³⁵ above and n is 0-4 or 1-4, said process comprising: oxidizing a compound of formula (YXII):

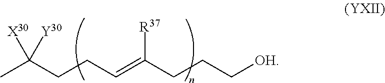

In still further aspects, a process for preparing a compound of formula (YXII)

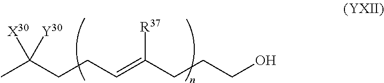

said process comprising:
reducing a compound of formula (YXIII)

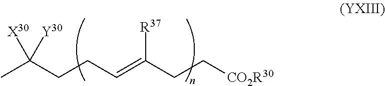

wherein R³⁰ is C₁-C₆ alkyl.

In further aspects, a process for preparing a compound of formula (YXIII):

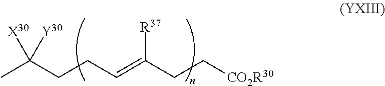

said process comprising:
contacting an orthoacetate of formula CH₃—C(OR³⁰)₃ wherein R⁴⁰ is C₁-C₆ alkyl with a compound of formula (YXIV):

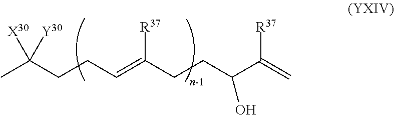

In further aspects, a process for preparing a compound of formula (YXIV):

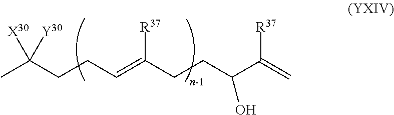

said process comprising:
contacting of a compound of formula (YXV):

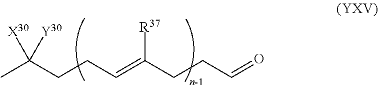

with a compound of formula

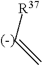

In each of the embodiments above, X³⁰ and Y³⁰ are as defined in formula (XXXII) above, R³⁷ is independently hydrogen or C₁-C₆ alkyl, n is 1-5, R³⁰ is hydrogen or C₁-C₆ alkyl, preferably an alkyl group.

### DETAILED DESCRIPTION OF THE INVENTION

### Definitions

This invention relates to compositions of GGA and GGA derivatives co-crystallized with urea or thiourea and processes related thereto. Before describing this invention in detail, the following terms will be defined.

It must be noted that as used herein and in the appended claims, the singular forms “a”, “an”, and “the” include plural referents unless the context clearly dictates otherwise. Thus, for example, reference to “a solvent” includes a plurality of such solvents.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition or process consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques. The term “about” when used before a numerical designation, e.g., temperature, time, amount, and concentration, including range, indicates approximations which may vary by (+) or (−) 10%, 5% or 1%.

As used herein, Cₘ-Cₙ, such as C₁-C₁₀, C₁-C₆, or C₁-C₄ when used before a group refers to that group containing m to n carbon atoms.

Geranylgeranyl acetone (GGA) refers to a compound of the formula:

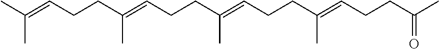 [SMILES: CC(=O)CC/C=C(\C)CC/C=C(\C)CC/C=C(\C)CCC=C(C)C]

wherein compositions comprising the compound are mixtures of geometrical isomers of the compound.

The 5-trans isomer of geranylgeranyl acetone refers to a compound of the formula:

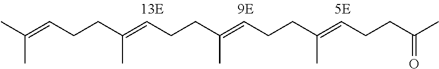 [SMILES: CC(=O)CC/C=C(\C)CC/C=C(\C)CC/C=C(\C)CCC=C(C)C]

wherein the number 5 carbon atom is in the 5-trans (5E) configuration.

The 5-cis isomer of geranylgeranyl acetone refers to a compound of the formula:

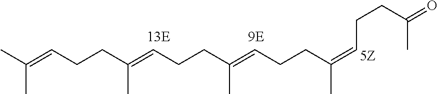 [SMILES: CC(=O)CC/C=C(/C)CC/C=C(\C)CC/C=C(\C)CCC=C(C)C]

wherein the number 5 carbon atom is in the 5-cis (5Z) configuration.

The term “alkoxy” refers to —O-alkyl.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

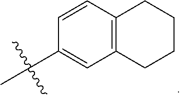 [SMILES: *c1ccc2c(c1)CCCC2]

The term “—CO₂H ester” refers to an ester formed between the —CO₂H group and an alcohol, preferably an aliphatic alcohol. A preferred example included —CO₂R^{E}, wherein R^{E} is alkyl or aryl group optionally substituted with an amino group.

“Co-crystal,” or as sometimes referred to herein “co-precipitate” refers to a solid, preferably a crystalline solid, comprising GGA or a GGA derivative, and urea or thiourea, more preferably, where, the GGA or the GGA derivative reside within the urea or thiourea lattice, such as in channels formed by urea or thiourea.

“Complexed” refers to GGA or a GGA derivative bound by certain quantifiable intermolecular forces, non-limiting examples of which include hydrogen bonding and Van-Der Waals' interactions, and also by entropic effects.

The term “chiral moiety” refers to a moiety that is chiral. Such a moiety can possess one or more asymmetric centers. Preferably, the chiral moiety is enantiomerically enriched, and more preferably a single enantiomer. Non limiting examples of chiral moieties include chiral carboxylic acids, chiral amines, chiral amino acids, such as the naturally occurring amino acids, chiral alcohols including chiral steroids, and the likes.

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

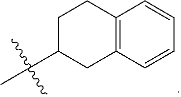 [SMILES: *C1CCc2ccccc2C1]

The term “halo” refers to F, Cl, Br, and/or I.

The term “heteroaryl” refers to a monovalent, aromatic mono-, bi-, or tricyclic ring having 2-14 ring carbon atoms and 1-6 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

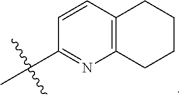 [SMILES: *c1ccc2c(n1)CCCC2]

The term “heterocyclyl” or heterocycle refers to a non-aromatic, mono-, bi-, or tricyclic ring containing 2-10 ring carbon atoms and 1-6 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that they ring is non-aromatic. Nonlimiting examples of heterocyclyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

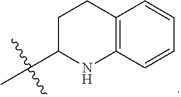 [SMILES: *C1CCc2ccccc2N1]

The term “hydrolyzing” refers to breaking an R^{H}—O—CO—, R^{H}—O—CS—, or an R^{H}—O—SO₂-moiety to an R^{H}—OH, preferably by adding water across the broken bond. A hydrolyzing is performed using various methods well known to the skilled artisan, non limiting examples of which include acidic and basic hydrolysis. A non-limiting example of hydrolyzing includes hydrolyzing a ketal, a thioketal and the likes to the corresponding ketone. A hydrolyzing is performed using various methods well known to the skilled artisan, non-limiting examples of which include acidic hydrolysis. A variety of acids such as protic acids and Lewis acids can be used for the hydrolysis.

The term “oxidizing” or “oxidation” refers to taking one or more electron away from a bond or an atom, preferably taking two electrons away from a bond or an atom. Non-limiting examples of oxidation include conversion of an alcohol to an aldehyde.

The term “oxo” refers to a C═O group, and to a substitution of 2 geminal hydrogen atoms with a C═O group.

The term “olefination” refers to conversion of a bond to the corresponding olefinic derivative. For example, without limitation, conversion of C═O to C═CR^{x}R^{y}, wherein R^{x} and R^{y} are alkyl groups.

The term “pharmaceutically acceptable” refers to safe and non-toxic for in vivo, preferably, human administration.

The term “pharmaceutically acceptable salt” refers to a salt that is pharmaceutically acceptable.

The term “reducing” or “reduction” refers to adding one or more electron across a bond or an atom, preferably adding two electrons to a bond or an atom. Non-limiting examples of reduction include conversion of a carboxylic acid or an ester thereof to an alcohol.

The term “salt” refers to an ionic compound formed between an acid and a base. When the compound provided herein contains an acidic functionality, such salts include, without limitation, alkai metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionally, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisalfate, mono-, di-, and tribasic phosphate, mesylate, tosylate, and the likes.

The term “substantially pure trans isomer” or grammatical equivalents thereof refers to a trans isomer that is by molar amount 95%, preferably 96%, more preferably 99%, and still more preferably 99.5% or more a trans isomer with the rest being the corresponding cis isomer. The term “substantially pure cis isomer” or grammatical equivalents thereof refers to a cis isomer that is by molar amount 95%, preferably 96%, more preferably 99%, and still more preferably 99.5% or more a cis isomer with the rest being the corresponding trans isomer.

“Trans” in the context of GGA and GGA derivatives refer to the GGA scaffold as illustrated below:

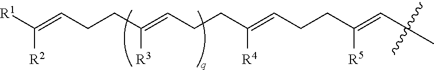

wherein R¹-R⁵ is defined herein and q is 0-2. As shown, each double bond is in a trans or E configuration. In contrast, a cis form of GGA or a GGA derivative will contain one or more of these bonds in a cis or Z configuration.

### GGA Derivatives

GGA derivatives useful in this invention include those described in U.S. patent application Ser. No. 13/815,831, US Patent Application Publication No. US 2006/0052347, U.S. Pat. No. 5,453,524, PCT Publication Nos. WO 2012/026813, WO 2012/031028 WO/2013/052148, WO/2013/130648, WO/2013/130242, and WO/2013/130654, each of which are incorporated herein by reference in its entirety. These and other GGA derivatives utilized herein are structurally shown below.

In one aspect, the GGA derivative utilized herein is of Formula I:

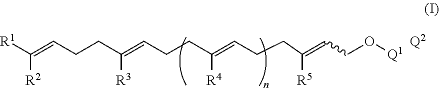

wherein
n is 1 or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups; each of R³, R⁴, and R⁵ independently are hydrogen or C₁-C₆ alkyl;

### Q¹ is —(C═O)—, —(C═S)—, or —S(O₂)—;

Q² is hydrogen, R⁶, —O—R⁶, —NR⁷R⁸, or is a chiral moiety;

### R⁶ is:

C₁-C₆ alkyl, optionally substituted with —CO₂H or an ester thereof, C₁ alkoxy, oxo, —CR═CR₂, —C≡CR, C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups, optionally substituted with 1-3 halo, preferably, fluoro, groups, or R₁₈ and R₁₉ together with the nitrogen atom to which they are attached to form a 5-7 membered heterocycle; each R⁷ and R⁸ are independently hydrogen or defined as R⁶; [symbol]
refers to a mixture of cis and trans isomers at the corresponding position wherein at least 80% and, preferably, no more than 95% of the compound of Formula (I) is present as a trans isomer;
or a salt or a tautomer thereof.

In one embodiment, the GGA derivative utilized is of Formula (I-A):

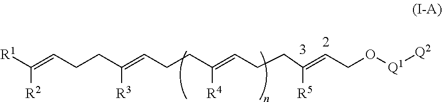

as a substantially pure trans isomer around the 2,3 double bond wherein, n, R¹-R⁵, Q¹, and Q² are defined as in Formula (I) above.

In another embodiment, n is 1. In another embodiment, n is 2.

In another embodiment, the GGA derivative utilized is of Formula (I-B):

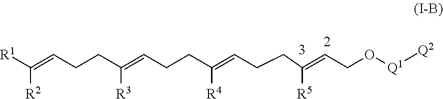

as a substantially pure trans isomer around the 2,3 double bond wherein, R¹-R⁵, Q¹, and Q² are defined as in Formula (I) above.

In another embodiment, the GGA derivative utilized is of Formula II:

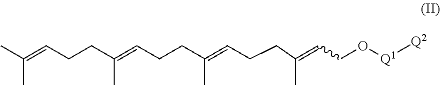

wherein Q¹ and Q² are defined as in Formula (I) above.

In another embodiment, the GGA derivative utilized is of Formula (II-A), (II-B), or (II-C):

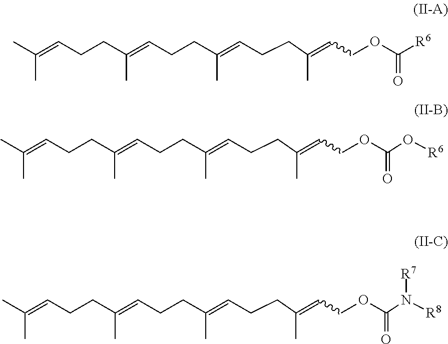

wherein R⁶-R⁸ are defined as in Formula (I) above.

In another embodiment, the GGA derivative utilized is of Formula (II-D), (II-E), or (II-F):

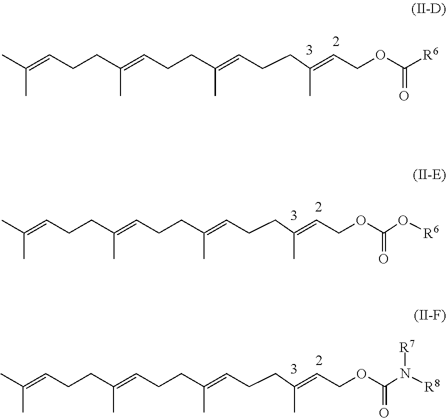

as a substantially pure trans isomer around the 2,3 double bond wherein R⁶-R⁸ are defined as in Formula (I) above.

In a preferred embodiment, R⁶ is C₆-C₁₀ aryl, such as naphthyl. In another preferred embodiment, R⁶ is a heteroaryl, such as quinolinyl.

In another aspect, the GGA derivative utilized in this invention is of Formula (II):

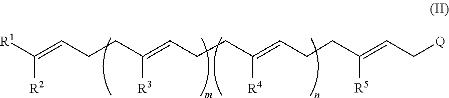

or a pharmaceutically acceptable salt thereof, wherein m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;

### Q is —X—CO—NR¹⁸R¹⁹, —X—CS—NR¹⁸R¹⁹, or —X—SO₂—NR¹⁸R¹⁹;

### X is —O—, —S—, —NR⁷—, or —CR⁸R⁹;

R⁷ is hydrogen or together with R¹⁸ or R¹⁹ and the intervening atoms form a 5-7 membered heterocyclic ring optionally substituted with 1-3 C₁-C₆ alkyl groups;
each R⁸ and R⁹ independently are hydrogen, C₁-C₆ alkyl, —COR⁸¹ or —CO₂R⁸¹, or R⁸ together with R¹⁸ or R¹⁹ and the intervening atoms form a 5-7 membered heterocyclyl ring optionally substituted with 1-3 C₁-C₆ alkyl groups; and
each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, or R₁₈ and R₁₉ together with the nitrogen atom they are attached to form a 5-7 membered heterocycle.

As used herein, the compound of Formula (II) includes optical isomers such as enantiomers and diastereomers. As also used herein, an ester refers preferably to a phenyl or a C₁-C₆ alkyl ester, which phenyl or alkyl group is optionally substituted with a amino group.

In one embodiment, the compound of Formula (II) is of formula:

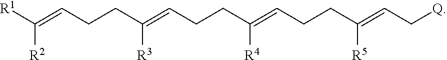

wherein R¹, R², R³, R⁴, R⁵, and Q are defined as in any aspect or embodiment herein.

In another embodiment, the GGA derivative utilized is of formula:

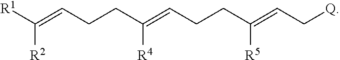

wherein R¹, R², R⁴, R⁵, and Q are defined as in any aspect and embodiment herein.

In one embodiment, the compound of Formula (II) is of formula:

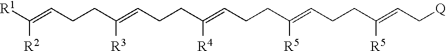

wherein R¹, R², R³, R⁴, R⁵, and Q are defined as in any aspect or embodiment herein.

In another embodiment, the GGA derivative utilized is of formula:

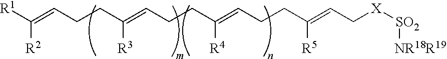

wherein R¹, R², R⁴, R⁵, m, n, X, R¹⁸ and R¹⁹ are defined as in any aspect and embodiment herein.

In another embodiment, the GGA derivative utilized is of formula:

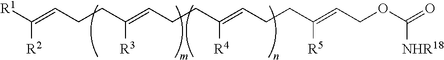

wherein R¹, R², R⁴, R⁵, m, n, and R¹⁸ are defined as in any aspect and embodiment herein.

In another embodiment, the GGA derivative utilized is of formula:

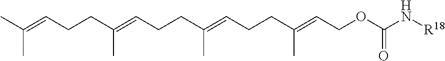

wherein R¹⁸ is defined as in any aspect and embodiment herein.

In another embodiment, the GGA derivative utilized is of formula:

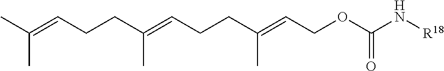

wherein R¹⁸ is defined as in any aspect and embodiment herein.

In another embodiment, the GGA derivative utilized is of formula:

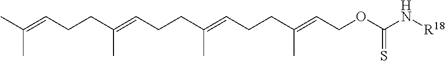

wherein R¹⁸ is defined as in any aspect and embodiment herein.

In another embodiment, the GGA derivative utilized is of formula:

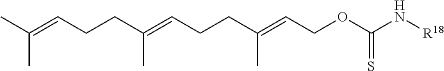

wherein R¹⁸ is defined as in any aspect and embodiment herein.

In another embodiment, the GGA derivative utilized is of formula:

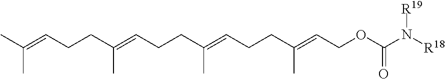

wherein R¹⁸ and R¹⁹ are defined as in any aspect and embodiment herein.

In another embodiment, the GGA derivative utilized is of formula:

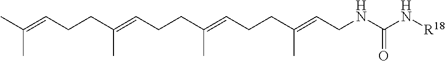

wherein R¹⁸ is defined as in any aspect and embodiment herein.

In another embodiment, the GGA derivative utilized is of formula:

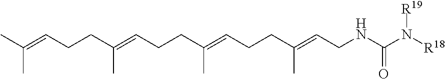

wherein R¹⁸ and R¹⁹ are defined as in any aspect and embodiment herein.

In one embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

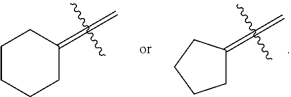 [SMILES: *=C1CCCCC1 ; *=C1CCCC1]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, Q is —X—CO—NR¹⁸R¹⁹. In another embodiment, Q is —X—CS—NR¹⁸R¹⁹. In another embodiment, Q is —X—SO₂—NR¹⁸R¹⁹. In another embodiment, Q is —OCONHR¹⁸, —OCONR¹⁸R¹⁹, NHCONHR¹⁸, NHCONR¹⁸R¹⁹, —OCSNHR¹⁸, —OCSNR¹⁸R¹⁹, —NHCSNHR¹⁸, or —NHCSNR¹⁸R¹⁹.

In another embodiment, X is —O—. In another embodiment, X is —NR⁷—. In another embodiment, X is or —CR⁸R⁹.

In another embodiment, one of R¹⁸ and R¹⁹ is hydrogen. In another embodiment, one or both of R¹⁸ and R¹⁹ are C₁-C₆ alkyl. In another embodiment, one or both of R¹⁸ and R¹⁹ are C₁-C₆ alkyl, optionally substituted with an R²⁰ group. In another embodiment, one or both of R¹⁸ and R¹⁹ are C₃-C₁₀ cycloalkyl. In another embodiment, one or both of R¹⁸ and R¹⁹ are C₃-C₁₀ cycloalkyl substituted with 1-3 alkyl groups. In another embodiment, one or both of R¹⁸ and R¹⁹ are C₃-C₈ heterocyclyl. In another embodiment, one or both of R¹⁸ and R¹⁹ are C₆-C₁₀ aryl. In another embodiment, one or both of R¹⁸ and R¹⁹ are C₂-C₁₀ heteroaryl. In another embodiment, R₁₈ and R₁₉ together with the nitrogen atom they are attached to form a 5-7 membered heterocycle.

In another embodiment, R²⁰ is —CO₂H or an ester thereof. In another embodiment, R²⁰ is C₃-C₈. In another embodiment, R²⁰ is cycloalkyl. In another embodiment, R²⁰ is C₃-C₈ heterocyclyl. In another embodiment, R²⁰ is C₆-C₁₀ aryl. In another embodiment, R²⁰ is or C₂-C₁₀ heteroaryl.

In another embodiment, examples of compounds utilized by this invention include certain compounds tabulated below.

Chemical Structure

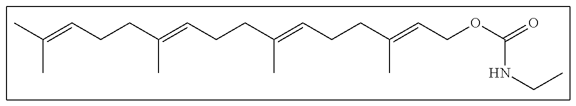 [SMILES: CCNC(=O)OC/C=C(\C)CC/C=C(\C)CC/C=C(\C)CCC=C(C)C]

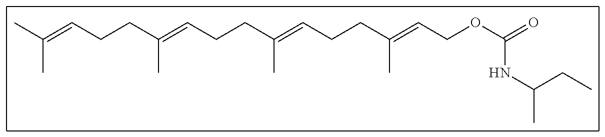 [SMILES: CCC(C)NC(=O)OC/C=C(\C)CC/C=C(\C)CC/C=C(\C)CCC=C(C)C]

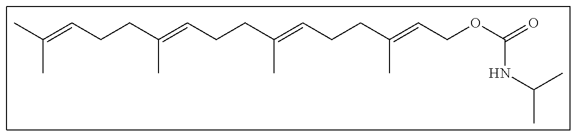 [SMILES: CC(C)=CCC/C(C)=C/CC/C(C)=C/CC/C(C)=C/COC(=O)NC(C)C]

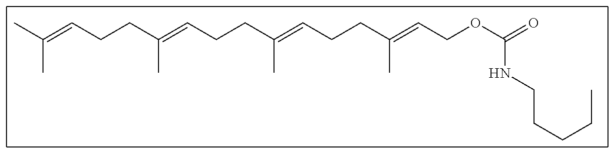 [SMILES: CCCCCNC(=O)OC/C=C(\C)CC/C=C(\C)CC/C=C(\C)CCC=C(C)C]

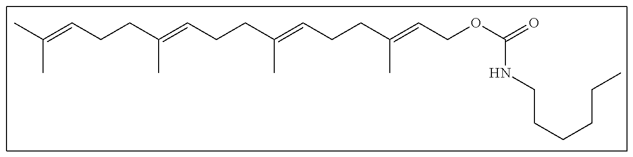 [SMILES: CCCCCCNC(=O)OC/C=C(\C)CC/C=C(\C)CC/C=C(\C)CCC=C(C)C]

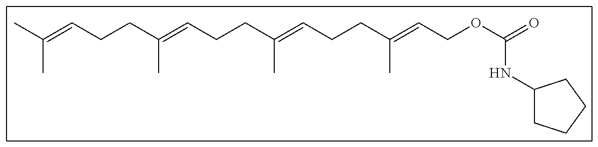 [SMILES: CC(C)=CCC/C(C)=C/CC/C(C)=C/CC/C(C)=C/COC(=O)NC1CCCC1]

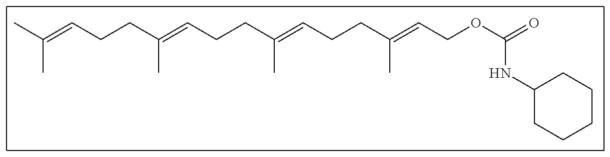 [SMILES: CC(C)=CCC/C(C)=C/CC/C(C)=C/CC/C(C)=C/COC(=O)NC1CCCCC1]

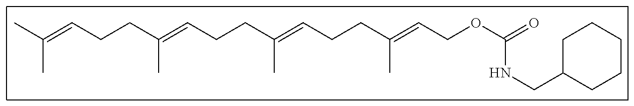 [SMILES: CC(C)=CCC/C(C)=C/CC/C(C)=C/CC/C(C)=C/COC(=O)NCC1CCCCC1]

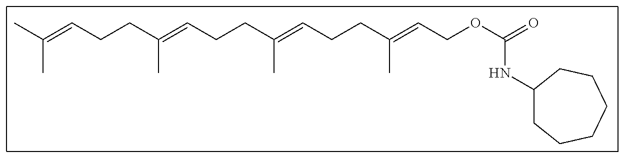 [SMILES: CC(C)=CCC/C(C)=C/CC/C(C)=C/CC/C(C)=C/COC(=O)NC1CCCCCC1]

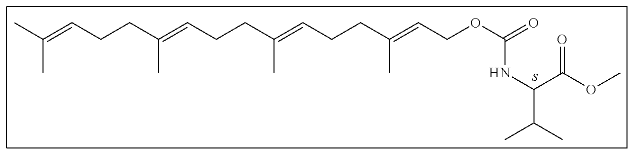

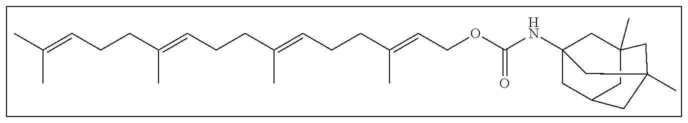 [SMILES: CC(C)=CCC/C(C)=C/CC/C(C)=C/CC/C(C)=C/COC(=O)NC12CC3CC(C)(CC(C)(C3)C1)C2]

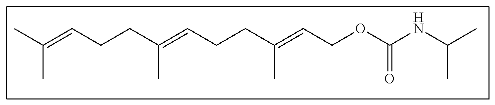 [SMILES: CC(C)=CCC/C(C)=C/CC/C(C)=C/COC(=O)NC(C)C]

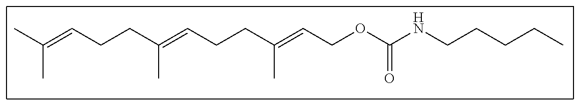 [SMILES: CCCCCNC(=O)OC/C=C(\C)CC/C=C(\C)CCC=C(C)C]

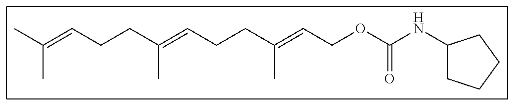 [SMILES: CC(C)=CCC/C(C)=C/CC/C(C)=C/COC(=O)NC1CCCC1]

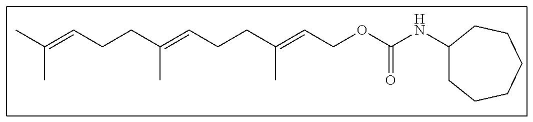 [SMILES: CC(C)=CCC/C(C)=C/CC/C(C)=C/COC(=O)NC1CCCCCC1]

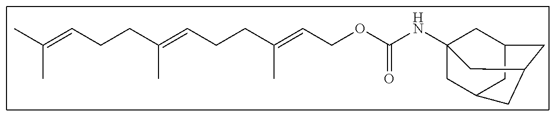 [SMILES: CC(C)=CCC/C(C)=C/CC/C(C)=C/COC(=O)NC12CC3CC(CC(C3)C1)C2]

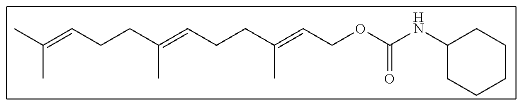 [SMILES: CC(C)=CCC/C(C)=C/CC/C(C)=C/COC(=O)NC1CCCCC1]

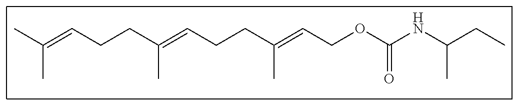 [SMILES: CCC(C)NC(=O)OC/C=C(\C)CC/C=C(\C)CCC=C(C)C]

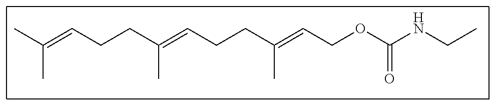 [SMILES: CCNC(=O)OC/C=C(\C)CC/C=C(\C)CCC=C(C)C]

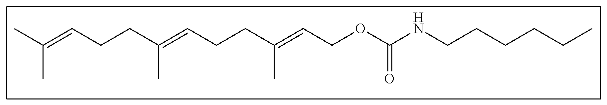 [SMILES: CCCCCCNC(=O)OC/C=C(\C)CC/C=C(\C)CCC=C(C)C]

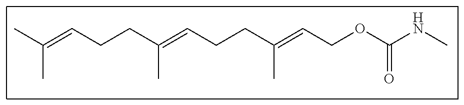 [SMILES: CNC(=O)OC/C=C(\C)CC/C=C(\C)CCC=C(C)C]

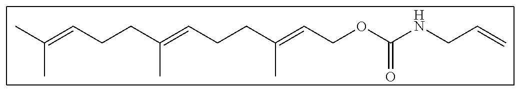 [SMILES: C=CCNC(=O)OC/C=C(\C)CC/C=C(\C)CCC=C(C)C]

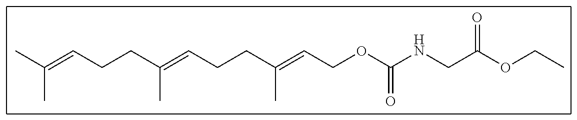 [SMILES: CCOC(=O)CNC(=O)OC/C=C(\C)CC/C=C(\C)CCC=C(C)C]

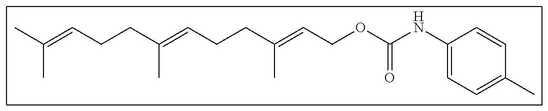 [SMILES: CC(C)=CCC/C(C)=C/CC/C(C)=C/COC(=O)Nc1ccc(C)cc1]

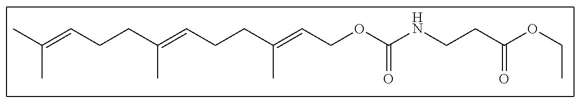 [SMILES: CCOC(=O)CCNC(=O)OC/C=C(\C)CC/C=C(\C)CCC=C(C)C]

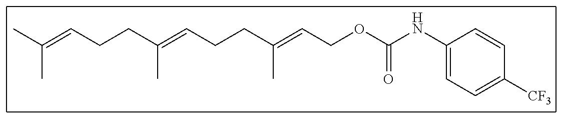 [SMILES: CC(C)=CCC/C(C)=C/CC/C(C)=C/COC(=O)Nc1ccc(C(F)(F)F)cc1]

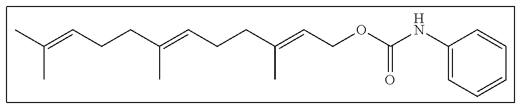 [SMILES: CC(C)=CCC/C(C)=C/CC/C(C)=C/COC(=O)Nc1ccccc1]

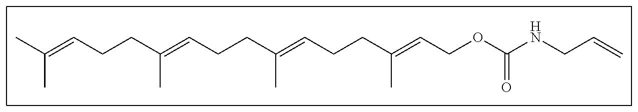 [SMILES: C=CCNC(=O)OC/C=C(\C)CC/C=C(\C)CC/C=C(\C)CCC=C(C)C]

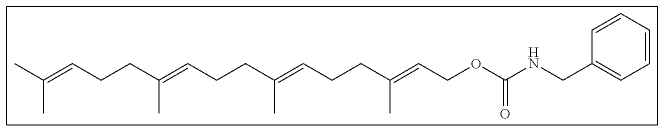 [SMILES: CC(C)=CCC/C(C)=C/CC/C(C)=C/CC/C(C)=C/COC(=O)NCc1ccccc1]

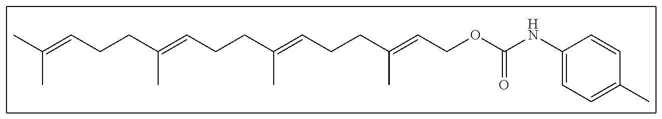 [SMILES: CC(C)=CCC/C(C)=C/CC/C(C)=C/CC/C(C)=C/COC(=O)Nc1ccc(C)cc1]

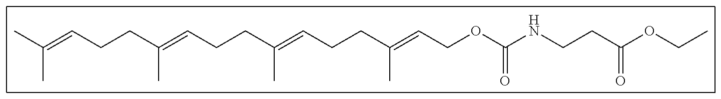 [SMILES: CCOC(=O)CCNC(=O)OC/C=C(\C)CC/C=C(\C)CC/C=C(\C)CCC=C(C)C]

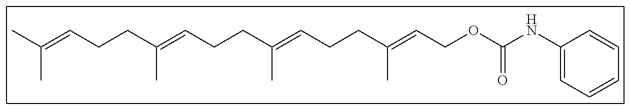 [SMILES: CC(C)=CCC/C(C)=C/CC/C(C)=C/CC/C(C)=C/COC(=O)Nc1ccccc1]

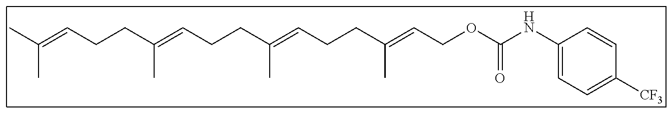 [SMILES: CC(C)=CCC/C(C)=C/CC/C(C)=C/CC/C(C)=C/COC(=O)Nc1ccc(C(F)(F)F)cc1]

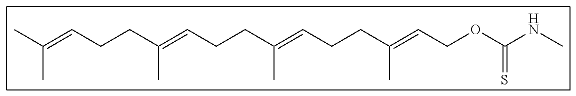 [SMILES: CNC(=S)OC/C=C(\C)CC/C=C(\C)CC/C=C(\C)CCC=C(C)C]

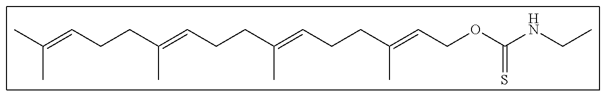 [SMILES: CCNC(=S)OC/C=C(\C)CC/C=C(\C)CC/C=C(\C)CCC=C(C)C]

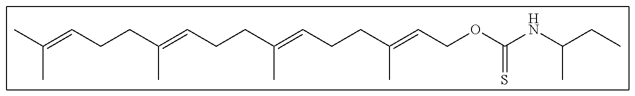 [SMILES: CCC(C)NC(=S)OC/C=C(\C)CC/C=C(\C)CC/C=C(\C)CCC=C(C)C]

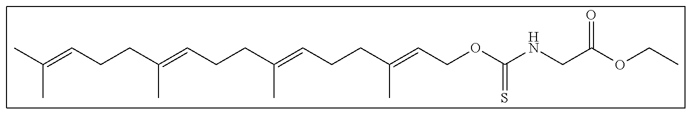 [SMILES: CCOC(=O)CNC(=S)OC/C=C(\C)CC/C=C(\C)CC/C=C(\C)CCC=C(C)C]

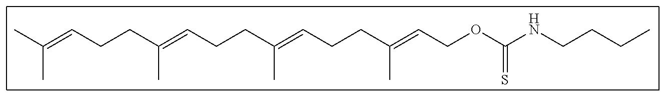 [SMILES: CCCCNC(=S)OC/C=C(\C)CC/C=C(\C)CC/C=C(\C)CCC=C(C)C]

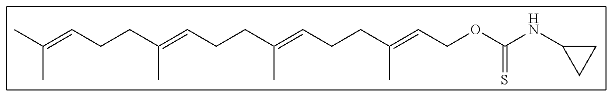 [SMILES: CC(C)=CCC/C(C)=C/CC/C(C)=C/CC/C(C)=C/COC(=S)NC1CC1]

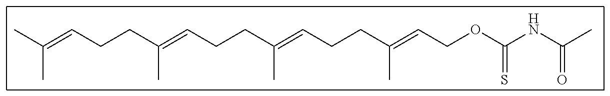 [SMILES: CC(=O)NC(=S)OC/C=C(\C)CC/C=C(\C)CC/C=C(\C)CCC=C(C)C]

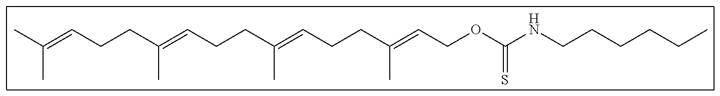 [SMILES: CCCCCCNC(=S)OC/C=C(\C)CC/C=C(\C)CC/C=C(\C)CCC=C(C)C]

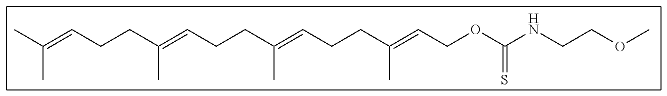 [SMILES: COCCNC(=S)OC/C=C(\C)CC/C=C(\C)CC/C=C(\C)CCC=C(C)C]

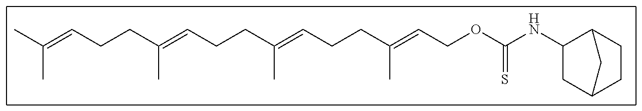 [SMILES: CC(C)=CCC/C(C)=C/CC/C(C)=C/CC/C(C)=C/COC(=S)NC1CC2CCC1C2]

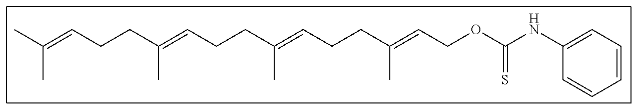 [SMILES: CC(C)=CCC/C(C)=C/CC/C(C)=C/CC/C(C)=C/COC(=S)Nc1ccccc1]

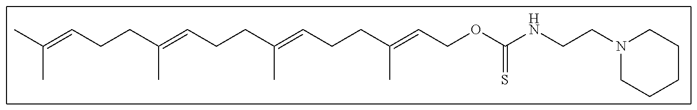 [SMILES: CC(C)=CCC/C(C)=C/CC/C(C)=C/CC/C(C)=C/COC(=S)NCCN1CCCCC1]

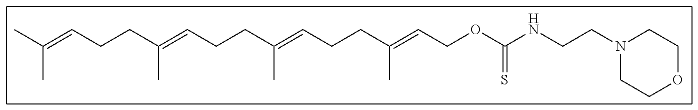 [SMILES: CC(C)=CCC/C(C)=C/CC/C(C)=C/CC/C(C)=C/COC(=S)NCCN1CCOCC1]

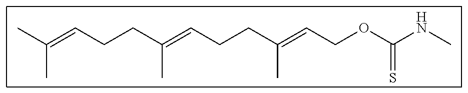 [SMILES: CNC(=S)OC/C=C(\C)CC/C=C(\C)CCC=C(C)C]

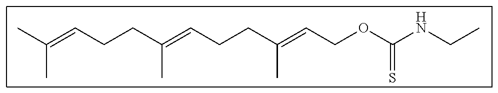 [SMILES: CCNC(=S)OC/C=C(\C)CC/C=C(\C)CCC=C(C)C]

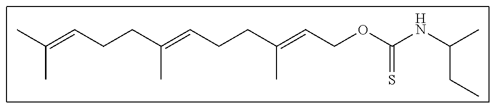 [SMILES: CCC(C)NC(=S)OC/C=C(\C)CC/C=C(\C)CCC=C(C)C]

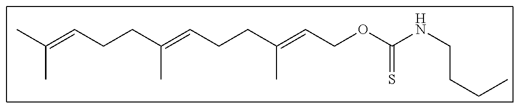 [SMILES: CCCCNC(=S)OC/C=C(\C)CC/C=C(\C)CCC=C(C)C]

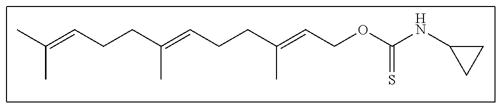 [SMILES: CC(C)=CCC/C(C)=C/CC/C(C)=C/COC(=S)NC1CC1]

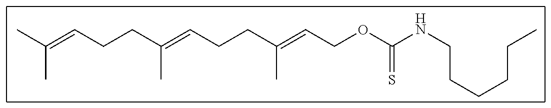 [SMILES: CCCCCCNC(=S)OC/C=C(\C)CC/C=C(\C)CCC=C(C)C]

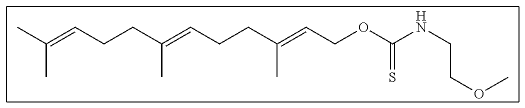 [SMILES: COCCNC(=S)OC/C=C(\C)CC/C=C(\C)CCC=C(C)C]

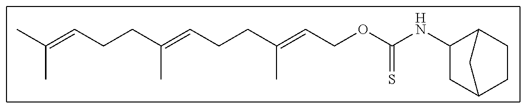 [SMILES: CC(C)=CCC/C(C)=C/CC/C(C)=C/COC(=S)NC1CC2CCC1C2]

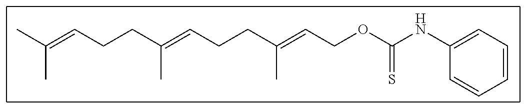 [SMILES: CC(C)=CCC/C(C)=C/CC/C(C)=C/COC(=S)Nc1ccccc1]

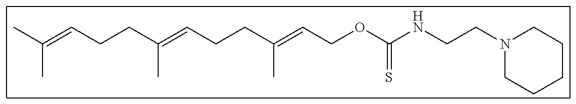 [SMILES: CC(C)=CCC/C(C)=C/CC/C(C)=C/COC(=S)NCCN1CCCCC1]

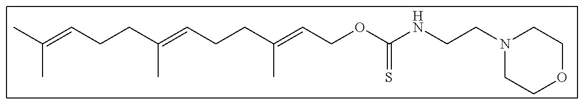 [SMILES: CC(C)=CCC/C(C)=C/CC/C(C)=C/COC(=S)NCCN1CCOCC1]

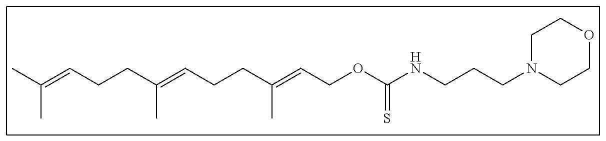 [SMILES: CC(C)=CCC/C(C)=C/CC/C(C)=C/COC(=S)NCCCN1CCOCC1]

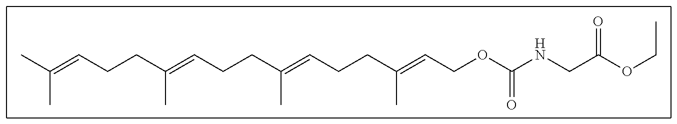 [SMILES: CCOC(=O)CNC(=O)OC/C=C(\C)CC/C=C(\C)CC/C=C(\C)CCC=C(C)C]

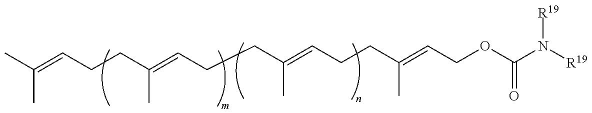

m = 0, n = 1, R¹⁸ = cyclohexyl, and R¹⁹ = methyl
m = 1, n = 1, R¹⁸ = cyclohexyl, and R¹⁹ = methyl
m = 1, n = 2, R¹⁸ = cyclohexyl, and R¹⁹ = methyl
m = 0, n = 1, R¹⁸ = n-pentyl, and R¹⁹ = methyl
m = 1, n = 1, R¹⁸ = n-pentyl, and R¹⁹ = methyl
m = 1, n = 2, R¹⁸ = n-pentyl, and R¹⁹ = methyl

In another aspect, the GGA derivative utilized is of formula:

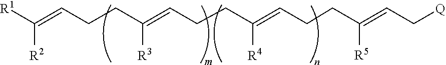

wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;
Q is —X—CO—NR¹⁸R¹⁹ or —X—SO₂—NR¹⁸R¹⁹;
X is —O—, —NR⁷—, or —CR⁸R⁹;
R⁷ is hydrogen or together with R¹⁸ or R¹⁹ and the intervening atoms form a 5-7 membered ring optionally substituted with 1-3 C₁-C₆ alkyl groups;
each R⁸ and R⁹ independently are hydrogen, C₁-C₆ alkyl, —COR⁸¹ or —CO₂R⁸¹, or R⁸ together with R¹⁸ or R¹⁹ and the intervening atoms form a 5-7 membered cycloalkyl or heterocyclyl ring optionally substituted with 1-3 C₁-C₆ alkyl groups;
each R¹⁸ and R¹⁹ independently is hydrogen, C₁-C₆ alkyl, optionally substituted with —CO₂H or an ester thereof, C₃-C₈ cycloalkyl, C₃-C₈ heterocyclyl, C₆-C₁₀ aryl, or is C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R₁₈ and R₁₉ together with the nitrogen atom they are attached to form a 5-7 membered heterocycle.

In another aspect, the GGA derivative utilized herein is of Formula III:

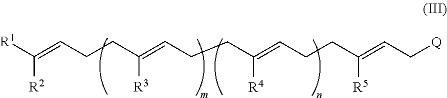

wherein

m is 0 or 1;

n is 0, 1, or 2;

each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;

each of R³, R⁴, and R⁵ independently are hydrogen or C₁-C₆ alkyl;

Q is selected from the group consisting of:

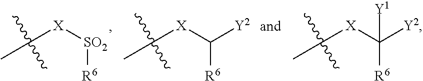

when X is bonded via a single bond, X is —O—, —NR⁷—, or —CR⁸R⁹—, and when X is bonded via a double bond, X is —CR⁸—;

Y¹ is hydrogen or —O—R¹⁰, Y² is —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹⁷);

R⁶ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups, or R⁶ is —NR¹⁸R¹⁹;

R⁷ is hydrogen or together with R⁶ and the intervening atoms form a 5-7 membered ring optionally substituted with 1-3 C₁-C₆ alkyl groups;

each R⁸ and R⁹ independently are hydrogen, C₁-C₆ alkyl, —COR⁸¹ or —CO₂R⁸¹, or R⁸ together with R⁶ and the intervening atoms form a 5-7 membered cycloalkyl or heterocyclyl ring optionally substituted with 1-3 C₁-C₆ alkyl groups;

R¹⁰ is C₁-C₆ alkyl;

R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a heterocycle optionally substituted with 1-3 C₁-C₆ alkyl groups;

R¹³ is C₁-C₆ alkyl or C₃-C₁₀cycloalkyl optionally substituted with 1-3 C₁-C₆ alkyl groups;

R¹⁴ is hydrogen, C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;

each R¹⁵ independently are hydrogen, C₃-C₁₀ cycloalkyl, C₁-C₆ alkyl optionally substituted with 1-3 substituents selected from the group consisting of —CO₂H or an ester thereof, aryl, or C₃-C₈ heterocyclyl, or two R¹⁵ groups together with the nitrogen atom they are bonded to form a 5-7 membered heterocycle;

R¹⁶ is hydrogen or C₁-C₆ alkyl;

R¹⁷ is hydrogen, C₁-C₆ alkyl substituted with 1-3 hydroxy groups, —CHO, or is CO₂H or an ester thereof;

each R¹⁸ and R¹⁹ independently is hydrogen, C₁-C₆ alkyl, optionally substituted with —CO₂H or an ester thereof, C₃-C₁₀ cycloalkyl, C₃-C₈ heterocyclyl, C₆-C₁₀ aryl, or C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R₁₈ and R₁₉ together with the nitrogen atom they are attached to form a 5-7 membered heterocycle; and

each R⁸¹ independently is C₁-C₆ alkyl.

In one embodiment, m is 0. In another embodiment, m is 1. In another embodiment, n is 0. In another embodiment, n is 1. In another embodiment, n is 2.

In one embodiment, the compound of Formula (III) is of formula:

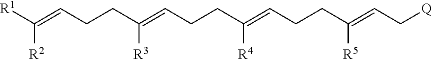

wherein R¹, R², R³, R⁴, R⁵, R⁶, X, Y¹, and Y² are defined as in any aspect or embodiment herein.

In one embodiment, the GGA derivative utilized is of formula:

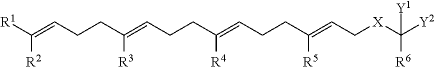

wherein R¹, R², R³, R⁴, R⁵, R⁶, X, Y¹, and Y² are defined as in any aspect and embodiment herein.

In another embodiment, the GGA derivative utilized is of formula:

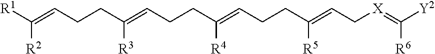

wherein R¹, R², R³, R⁴, R⁵, R⁶, X, and Y² are defined as in any aspect and embodiment herein.

In another embodiment, the GGA derivative utilized is of formula:

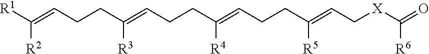

wherein R¹, R², R³, R⁴, R⁵, R⁶ and X are defined as in any aspect and embodiment herein.

In another embodiment, the GGA derivative utilized is of formula:

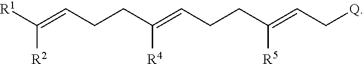

wherein R¹, R², R⁴, R⁵, and Q are defined as in any aspect and embodiment herein.

In another embodiment, the GGA derivative utilized is of formula:

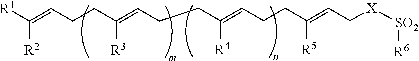

wherein R¹, R², R⁴, R⁵, m, n, X, and R⁶ are defined as in any aspect and embodiment herein.

In another embodiment, the GGA derivative utilized is of formula:

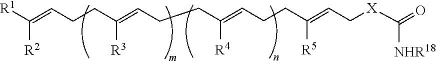

wherein R¹, R², R⁴, R⁵, m, n, and R¹⁸ are defined as in any aspect and embodiment herein.

In another embodiment, the GGA derivative utilized is of formula:

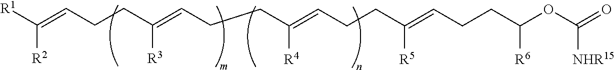

wherein R¹, R², R⁴, R⁵, R⁶, m, n, and R¹⁵ are defined as in any aspect and embodiment herein.

In another embodiment, each R¹ and R² are C₁-C₆ alkyl. In another embodiment, each R¹ and R² are methyl, ethyl, or isopropyl. In another embodiment, R¹ and R² together with the carbon atom they are attached to form a 5-6 membered ring optionally substituted with 1-3 C₁-C₆ alkyl groups. In another embodiment, R¹ and R² together with the carbon atom they are attached to form a ring that is:

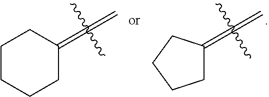 [SMILES: *=C1CCCCC1 ; *=C1CCCC1]

In another embodiment, R³, R⁴, and R⁵ are C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, X is O. In another embodiment, X is —NR⁷. In another embodiment, R⁷ is hydrogen. In another embodiment, R⁷ together with R⁶ and the intervening atoms form a 5-7 membered ring optionally substituted with 1-3 C₁-C₆ alkyl groups. In another embodiment, X is —CR⁸R⁹—. In another embodiment, X is —CR⁸—. In another embodiment, each R⁸ and R⁹ independently are hydrogen, C₁-C₆ alkyl, —COR⁸¹, or —CO₂R⁸¹. In another embodiment, R⁸ is hydrogen, and R⁹ is hydrogen, C₁-C₆ alkyl, —COR⁸¹, or —CO₂R⁸¹.

In another embodiment, R⁹ is hydrogen. In another embodiment, R⁹ C₁-C₆ alkyl. In another embodiment, R⁹ is methyl. In another embodiment, R⁹ is —CO₂R⁸¹. In another embodiment, R⁹ is —COR⁸¹.

In another embodiment, R⁸ together with R⁶ and the intervening atoms form a 5-7 membered ring. In another embodiment, the moiety:

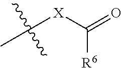

which is “Q,” has the structure:

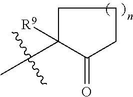

wherein R⁹ is hydrogen, C₁-C₆ alkyl, or —CO₂R⁸¹ and n is 1, 2, or 3. Within these embodiments, in certain embodiments, R⁹ is hydrogen or C₁-C₆ alkyl. In one embodiment, R⁹ is hydrogen. In another embodiment, R⁹ is C₁-C₆ alkyl.

In another embodiment, R⁶ is C₁-C₆ alkyl. In another embodiment, R⁶ is methyl, ethyl, butyl, isopropyl, or tertiary butyl. In another embodiment, R⁶ is C₁-C₆ alkyl substituted with 1-3 alkoxy or 1-5 halo group. In another embodiment, R⁶ is alkyl substituted with an alkoxy group. In another embodiment, R⁶ is alkyl substituted with 1-5, preferably, 1-3, halo, preferably fluoro, groups.

In another embodiment, R⁶ is NR¹⁸R¹⁹. In a preferred embodiment, R¹⁹ is H. In a preferred embodiment, R¹⁸ is C₁-C₆ alkyl, optionally substituted with a group selected from the group consisting of —CO₂H or an ester thereof, C₃-C₁₀ cycloalkyl, C₃-C₈ heterocyclyl, C₆-C₁₀ aryl, or C₂-C₁₀ heteroaryl. In another preferred embodiment, R¹⁸ is C₃-C₁₀ cycloalkyl, C₃-C₈ heterocyclyl, C₆-C₁₀ aryl, or C₂-C₁₀ heteroaryl. In a more preferred embodiment, R¹⁸ is C₃-C₁₀ cycloalkyl.

In another embodiment, R⁶ is C₂-C₆ alkenyl or C₂-C₆ alkynyl. In another embodiment, R⁶ is C₃-C₁₀ cycloalkyl. In another embodiment, R⁶ is C₃-C₁₀ cycloalkyl substituted with 1-3 C₁-C₆ alkyl groups. In another embodiment, R⁶ is cyclopropyl, cyclobutyl, cyclopentyl, cyclohexyl, or adamentyl. In another embodiment, R⁶ is C₆-C₁₀ aryl or C₂-C₁₀ heteroaryl. In another embodiment, R⁶ is a 5-7 membered heteroaryl containing at least 1 oxygen atom. In another embodiment, R⁶ is C₆-C₁₀ aryl, C₃-C₈ heterocyclyl, or C₂-C₁₀ heteroaryl, wherein each aryl, heterocyclyl, or heteroaryl is optionally substituted with 1-3 C₁-C₆ alkyl groups.

In another embodiment, Y² is —O—R¹¹. In another embodiment, Y¹ and Y² are joined to form ═NR¹³. In another embodiment, Y¹ and Y² are joined to form ═NOR¹⁴. In another embodiment, Y¹ and Y² are joined to form (═O). In another embodiment, Y¹ and Y² are joined to form ═CR¹⁶R¹⁷.

In another embodiment, Q is —CR⁹COR⁶. In another embodiment, R⁶ is C₁-C₆ alkyl optionally substituted with an alkoxy group. In another embodiment, R⁶ is C₃-C₈ cycloalkyl. In another embodiment, R⁹ is hydrogen. In another embodiment, R⁹ is C₁-C₆ alkyl. In another embodiment, R⁹ is CO₂R⁸¹. In another embodiment, R⁹ is COR⁸¹.

In another embodiment, Q is —CH₂—CH(O—CONHR¹⁵)—R⁶. In another embodiment, R¹⁵ is C₃-C₈ cycloalkyl. In another embodiment, R¹⁵ is C₁-C₆ alkyl optionally substituted with 1-3 substituents selected from the group consisting of —CO₂H or an ester thereof, aryl, or C₃-C₈ heterocyclyl. In a preferred embodiment within these embodiments, R⁶ is C₁-C₆ alkyl.

In another embodiment, Q is —O—CO—NHR¹⁸. Within these embodiments, in another embodiment, R¹⁸ is C₁-C₆ alkyl, optionally substituted with —CO₂H or an ester thereof, C₃-C₈ cycloalkyl, C₃-C₈ heterocyclyl, C₂-C₁₀ aryl, or C₂-C₁₀ heteroaryl. In yet another embodiment, R¹⁸ is C₃-C₈ cycloalkyl, C₃-C₈ heterocyclyl, C₂-C₁₀ aryl, or C₂-C₁₀ heteroaryl.

In another embodiment, R¹⁴ is hydrogen. In another embodiment, R¹⁴ is C₁-C₆ alkyl optionally substituted with a —CO₂H or an ester thereof or a C₆-C₁₀ aryl optionally substituted with 1-3 alkyl groups. In another embodiment, R¹⁴ is C₂-C₆ alkenyl. In another embodiment, R¹⁴ is C₂-C₆ alkynyl In another embodiment, R¹⁴ is C₃-C₆ cycloalkyl optionally substituted with 1-3 alkyl groups. In another embodiment, R¹⁴ is C₃-C₈ heterocyclyl optionally substituted with 1-3 alkyl groups.

In another embodiment, preferably, R¹⁶ is hydrogen. In another embodiment, R¹⁷ is CO₂H or an ester thereof. In another embodiment, R¹⁷ is C₁-C₆ alkyl substituted with 1-3 hydroxy groups. In another embodiment, R¹⁷ is C₁-C₃ alkyl substituted with 1 hydroxy group. In another embodiment, R¹⁷ is —CH₂OH.

In another embodiment, R¹⁰ and R¹¹ together with the intervening carbon atom and oxygen atoms form a heterocycle of formula:

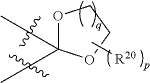

wherein q is 0 or 1, p is 0, 1, 2, or 3, and R²⁰ is C₁-C₆ alkyl.

In another embodiment, q is 1. In another embodiment, q is 2. In another embodiment, p is 0. In another embodiment, p is 1. In another embodiment, p is 2. In another embodiment, p is 3.

In another embodiment, examples of compounds utilized by this invention include certain compounds tabulated below.

TABLE 1
Chemical Structure

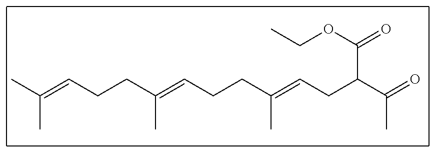 [SMILES: CCOC(=O)C(C/C=C(\C)CC/C=C(\C)CCC=C(C)C)C(C)=O]

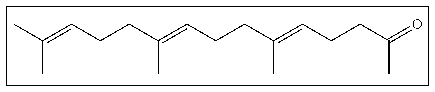 [SMILES: CC(=O)CC/C=C(\C)CC/C=C(\C)CCC=C(C)C]

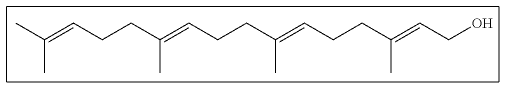 [SMILES: CC(C)=CCC/C(C)=C/CC/C(C)=C/CC/C(C)=C/CO]

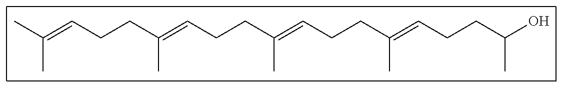 [SMILES: CC(C)=CCC/C(C)=C/CC/C(C)=C/CC/C(C)=C/CCC(C)O]

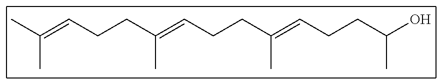 [SMILES: CC(C)=CCC/C(C)=C/CC/C(C)=C/CCC(C)O]

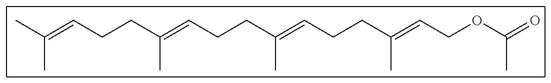 [SMILES: CC(=O)OC/C=C(\C)CC/C=C(\C)CC/C=C(\C)CCC=C(C)C]

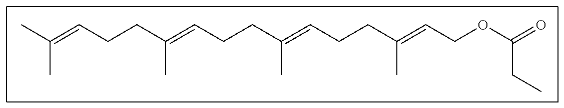 [SMILES: CCC(=O)OC/C=C(\C)CC/C=C(\C)CC/C=C(\C)CCC=C(C)C]

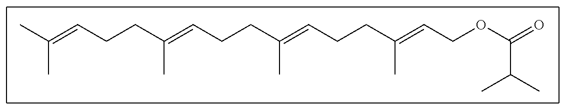 [SMILES: CC(C)=CCC/C(C)=C/CC/C(C)=C/CC/C(C)=C/COC(=O)C(C)C]

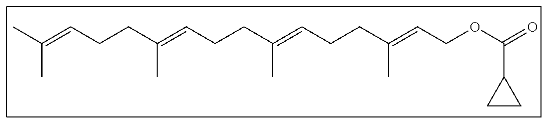 [SMILES: CC(C)=CCC/C(C)=C/CC/C(C)=C/CC/C(C)=C/COC(=O)C1CC1]

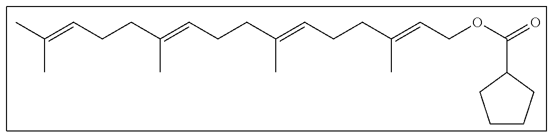 [SMILES: CC(C)=CCC/C(C)=C/CC/C(C)=C/CC/C(C)=C/COC(=O)C1CCCC1]

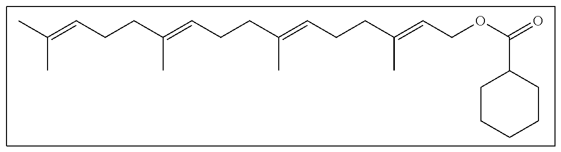 [SMILES: CC(C)=CCC/C(C)=C/CC/C(C)=C/CC/C(C)=C/COC(=O)C1CCCCC1]

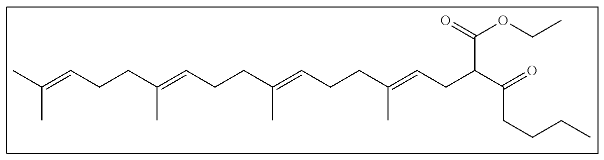 [SMILES: CCCCC(=O)C(C/C=C(\C)CC/C=C(\C)CC/C=C(\C)CCC=C(C)C)C(=O)OCC]

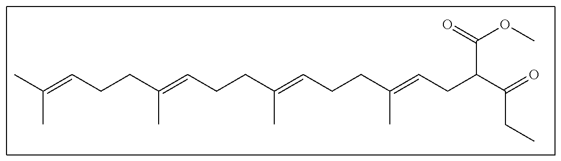 [SMILES: CCC(=O)C(C/C=C(\C)CC/C=C(\C)CC/C=C(\C)CCC=C(C)C)C(=O)OC]

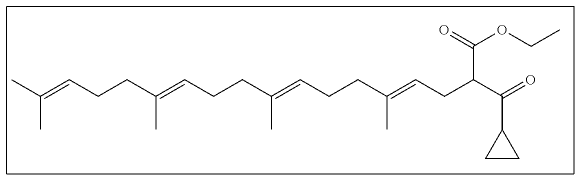 [SMILES: CCOC(=O)C(C/C=C(\C)CC/C=C(\C)CC/C=C(\C)CCC=C(C)C)C(=O)C1CC1]

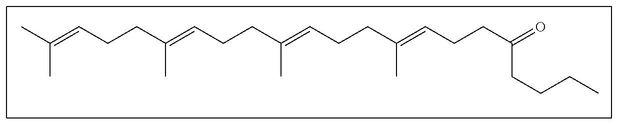 [SMILES: CCCCC(=O)CC/C=C(\C)CC/C=C(\C)CC/C=C(\C)CCC=C(C)C]

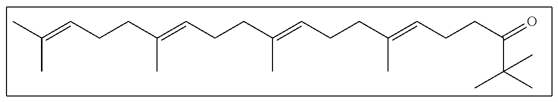 [SMILES: CC(C)=CCC/C(C)=C/CC/C(C)=C/CC/C(C)=C/CCC(=O)C(C)(C)C]

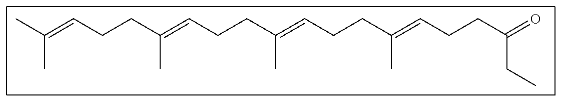 [SMILES: CCC(=O)CC/C=C(\C)CC/C=C(\C)CC/C=C(\C)CCC=C(C)C]

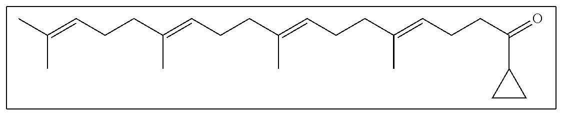 [SMILES: CC(C)=CCC/C(C)=C/CC/C(C)=C/CC/C(C)=C/CCC(=O)C1CC1]

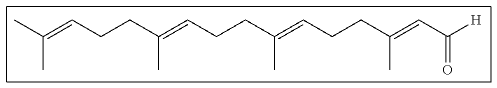 [SMILES: CC(C)=CCC/C(C)=C/CC/C(C)=C/CC/C(C)=C/C=O]

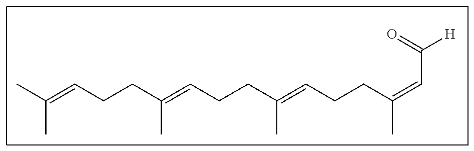 [SMILES: CC(C)=CCC/C(C)=C/CC/C(C)=C/CC/C(C)=C\C=O]

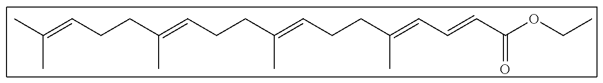 [SMILES: CCOC(=O)/C=C/C=C(\C)CC/C=C(\C)CC/C=C(\C)CCC=C(C)C]

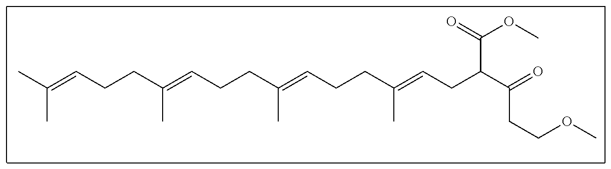 [SMILES: COCCC(=O)C(C/C=C(\C)CC/C=C(\C)CC/C=C(\C)CCC=C(C)C)C(=O)OC]

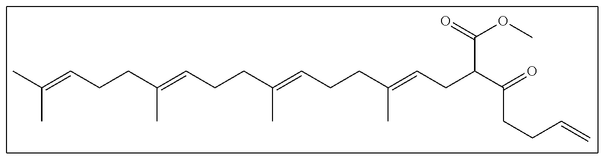 [SMILES: C=CCCC(=O)C(C/C=C(\C)CC/C=C(\C)CC/C=C(\C)CCC=C(C)C)C(=O)OC]

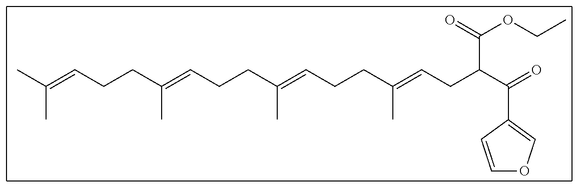 [SMILES: CCOC(=O)C(C/C=C(\C)CC/C=C(\C)CC/C=C(\C)CCC=C(C)C)C(=O)c1ccoc1]

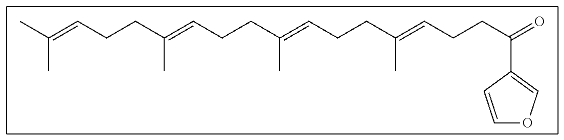 [SMILES: CC(C)=CCC/C(C)=C/CC/C(C)=C/CC/C(C)=C/CCC(=O)c1ccoc1]

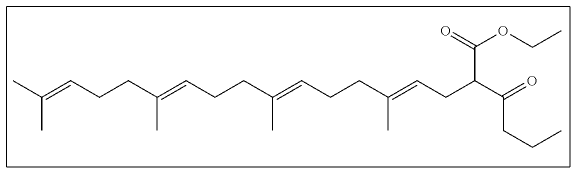 [SMILES: CCCC(=O)C(C/C=C(\C)CC/C=C(\C)CC/C=C(\C)CCC=C(C)C)C(=O)OCC]

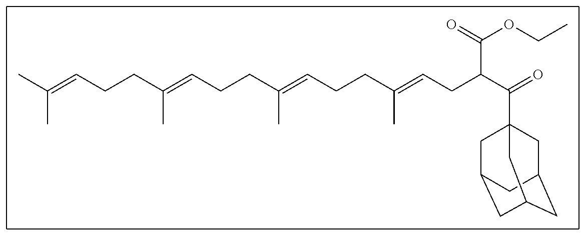 [SMILES: CCOC(=O)C(C/C=C(\C)CC/C=C(\C)CC/C=C(\C)CCC=C(C)C)C(=O)C12CC3CC(CC(C3)C1)C2]

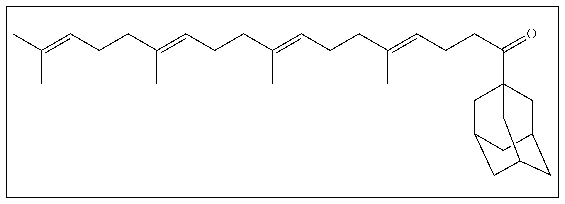 [SMILES: CC(C)=CCC/C(C)=C/CC/C(C)=C/CC/C(C)=C/CCC(=O)C12CC3CC(CC(C3)C1)C2]

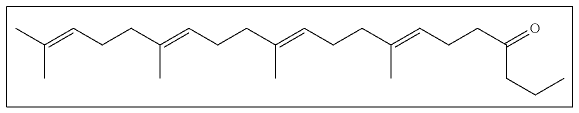 [SMILES: CCCC(=O)CC/C=C(\C)CC/C=C(\C)CC/C=C(\C)CCC=C(C)C]

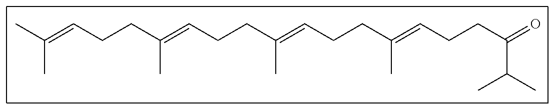 [SMILES: CC(C)=CCC/C(C)=C/CC/C(C)=C/CC/C(C)=C/CCC(=O)C(C)C]

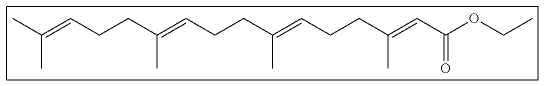 [SMILES: CCOC(=O)/C=C(\C)CC/C=C(\C)CC/C=C(\C)CCC=C(C)C]

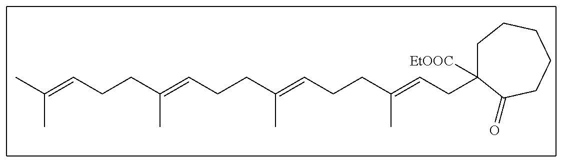 [SMILES: CCOC(=O)C1(C/C=C(\C)CC/C=C(\C)CC/C=C(\C)CCC=C(C)C)CCCCCC1=O]

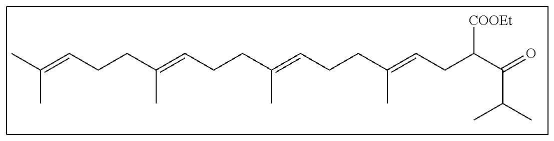 [SMILES: CCOC(=O)C(C/C=C(\C)CC/C=C(\C)CC/C=C(\C)CCC=C(C)C)C(=O)C(C)C]

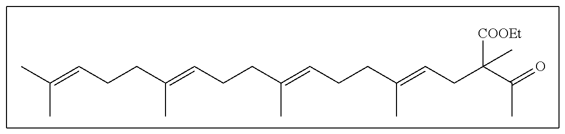 [SMILES: CCOC(=O)C(C)(C/C=C(\C)CC/C=C(\C)CC/C=C(\C)CCC=C(C)C)C(C)=O]

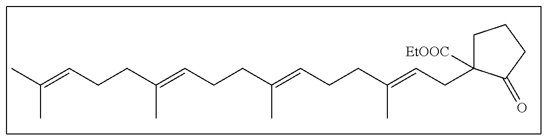 [SMILES: CCOC(=O)C1(C/C=C(\C)CC/C=C(\C)CC/C=C(\C)CCC=C(C)C)CCCC1=O]

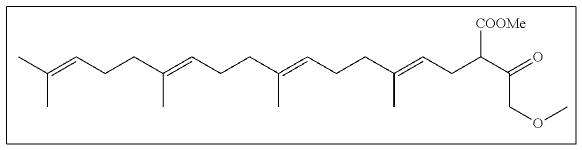 [SMILES: COCC(=O)C(C/C=C(\C)CC/C=C(\C)CC/C=C(\C)CCC=C(C)C)C(=O)OC]

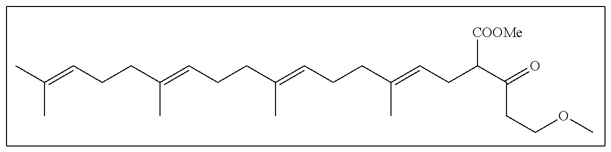 [SMILES: COCCC(=O)C(C/C=C(\C)CC/C=C(\C)CC/C=C(\C)CCC=C(C)C)C(=O)OC]

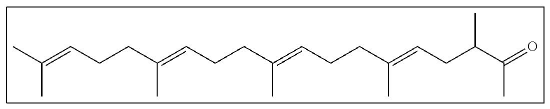 [SMILES: CC(=O)C(C)C/C=C(\C)CC/C=C(\C)CC/C=C(\C)CCC=C(C)C]

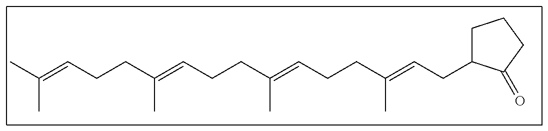 [SMILES: CC(C)=CCC/C(C)=C/CC/C(C)=C/CC/C(C)=C/CC1CCCC1=O]

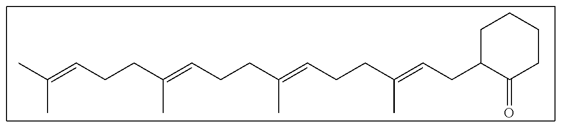 [SMILES: CC(C)=CCC/C(C)=C/CC/C(C)=C/CC/C(C)=C/CC1CCCCC1=O]

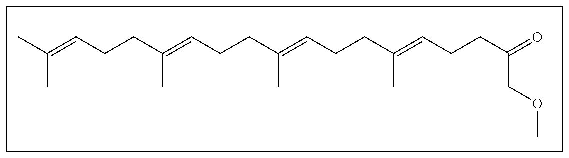 [SMILES: COCC(=O)CC/C=C(\C)CC/C=C(\C)CC/C=C(\C)CCC=C(C)C]

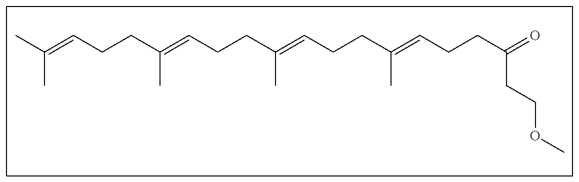 [SMILES: COCCC(=O)CC/C=C(\C)CC/C=C(\C)CC/C=C(\C)CCC=C(C)C]

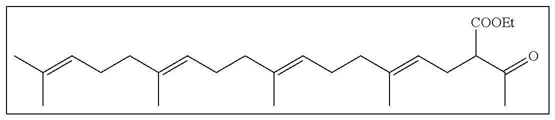 [SMILES: CCOC(=O)C(C/C=C(\C)CC/C=C(\C)CC/C=C(\C)CCC=C(C)C)C(C)=O]

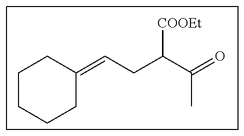 [SMILES: CCOC(=O)C(CC=C1CCCCC1)C(C)=O]

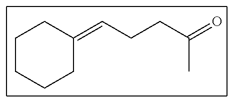

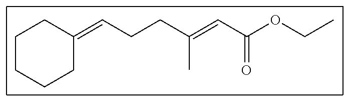 [SMILES: CCOC(=O)/C=C(\C)CCC=C1CCCCC1]

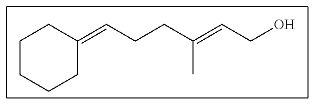

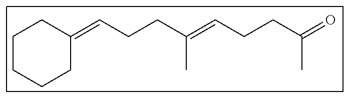 [SMILES: CC(=O)CC/C=C(\C)CCC=C1CCCCC1]

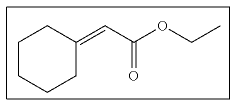

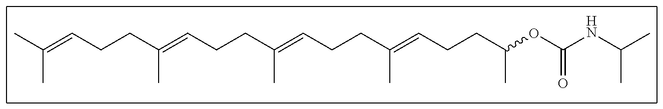 [SMILES: CC(C)=CCC/C(C)=C/CC/C(C)=C/CC/C(C)=C/CCC(C)OC(=O)NC(C)C]

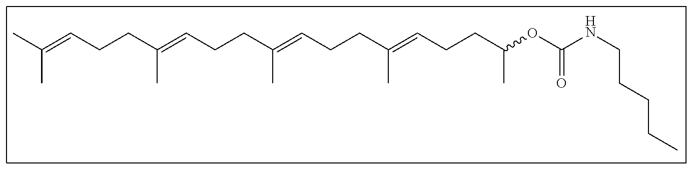 [SMILES: CCCCCNC(=O)OC(C)CC/C=C(\C)CC/C=C(\C)CC/C=C(\C)CCC=C(C)C]

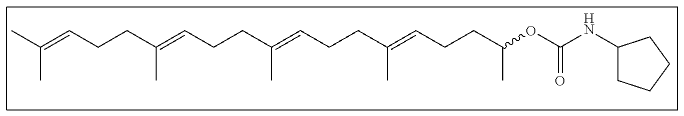 [SMILES: CC(C)=CCC/C(C)=C/CC/C(C)=C/CC/C(C)=C/CCC(C)OC(=O)NC1CCCC1]

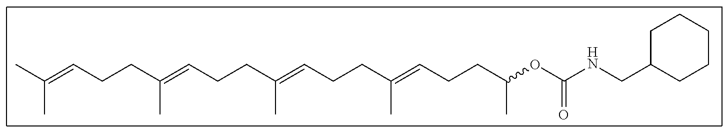 [SMILES: CC(C)=CCC/C(C)=C/CC/C(C)=C/CC/C(C)=C/CCC(C)OC(=O)NCC1CCCCC1]

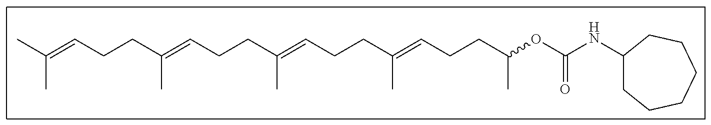 [SMILES: CC(C)=CCC/C(C)=C/CC/C(C)=C/CC/C(C)=C/CCC(C)OC(=O)NC1CCCCCC1]

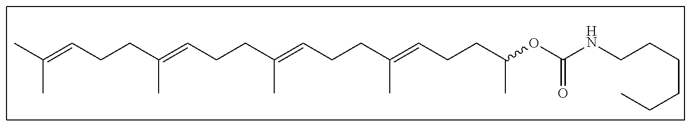 [SMILES: CCCCCCNC(=O)OC(C)CC/C=C(\C)CC/C=C(\C)CC/C=C(\C)CCC=C(C)C]

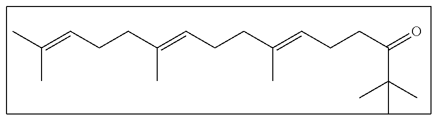 [SMILES: CC(C)=CCC/C(C)=C/CC/C(C)=C/CCC(=O)C(C)(C)C]

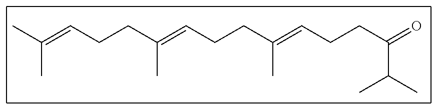 [SMILES: CC(C)=CCC/C(C)=C/CC/C(C)=C/CCC(=O)C(C)C]

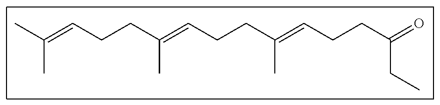 [SMILES: CCC(=O)CC/C=C(\C)CC/C=C(\C)CCC=C(C)C]

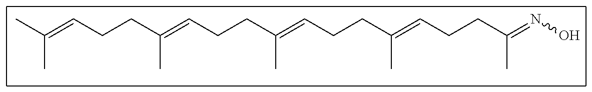 [SMILES: CC(C)=CCC/C(C)=C/CC/C(C)=C/CC/C(C)=C/CCC(C)=NO]

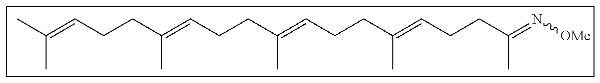 [SMILES: CON=C(C)CC/C=C(\C)CC/C=C(\C)CC/C=C(\C)CCC=C(C)C]

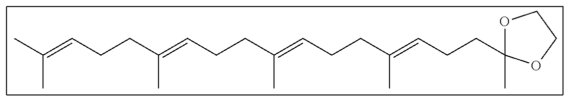 [SMILES: CC(C)=CCC/C(C)=C/CC/C(C)=C/CC/C(C)=C/CCC1(C)OCCO1]

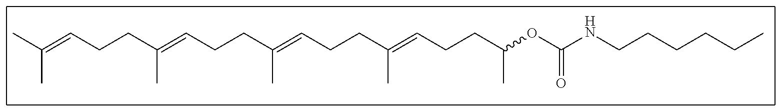 [SMILES: CCCCCCNC(=O)OC(C)CC/C=C(\C)CC/C=C(\C)CC/C=C(\C)CCC=C(C)C]

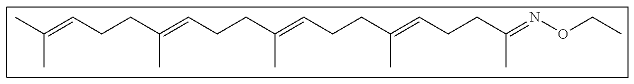 [SMILES: CCO/N=C(\C)CC/C=C(\C)CC/C=C(\C)CC/C=C(\C)CCC=C(C)C]

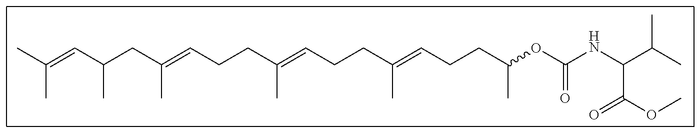 [SMILES: COC(=O)C(NC(=O)OC(C)CC/C=C(\C)CC/C=C(\C)CC/C=C(\C)CC(C)C=C(C)C)C(C)C]

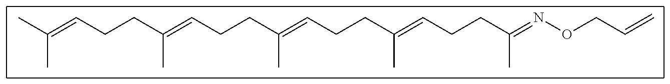 [SMILES: C=CCO/N=C(\C)CC/C=C(\C)CC/C=C(\C)CC/C=C(\C)CCC=C(C)C]

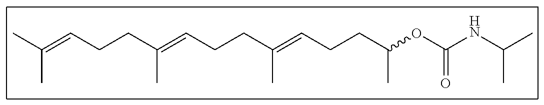 [SMILES: CC(C)=CCC/C(C)=C/CC/C(C)=C/CCC(C)OC(=O)NC(C)C]

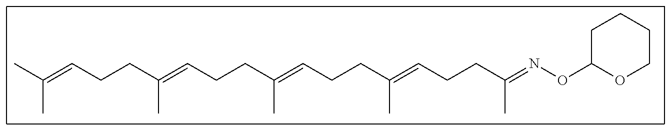 [SMILES: CC(C)=CCC/C(C)=C/CC/C(C)=C/CC/C(C)=C/CC/C(C)=N/OC1CCCCO1]

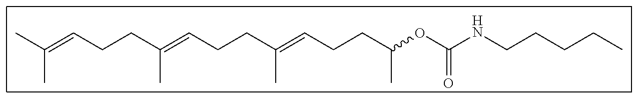 [SMILES: CCCCCNC(=O)OC(C)CC/C=C(\C)CC/C=C(\C)CCC=C(C)C]

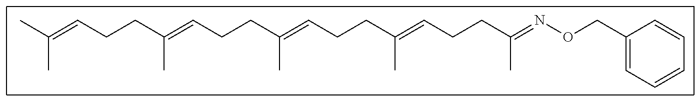 [SMILES: CC(C)=CCC/C(C)=C/CC/C(C)=C/CC/C(C)=C/CC/C(C)=N/OCc1ccccc1]

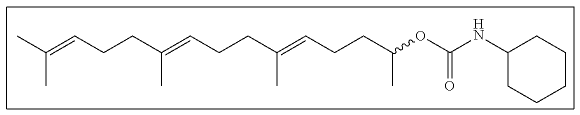 [SMILES: CC(C)=CCC/C(C)=C/CC/C(C)=C/CCC(C)OC(=O)NC1CCCCC1]

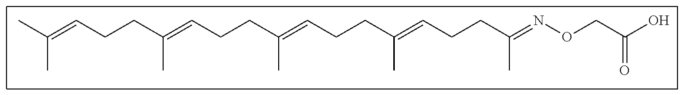 [SMILES: CC(C)=CCC/C(C)=C/CC/C(C)=C/CC/C(C)=C/CC/C(C)=N/OCC(=O)O]

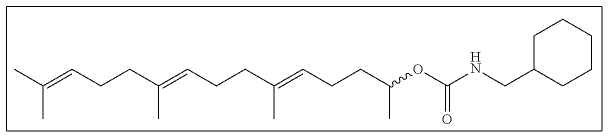 [SMILES: CC(C)=CCC/C(C)=C/CC/C(C)=C/CCC(C)OC(=O)NCC1CCCCC1]

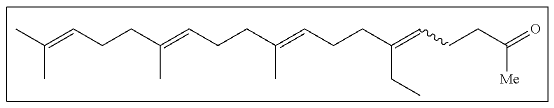 [SMILES: CCC(=CCCC(C)=O)CC/C=C(\C)CC/C=C(\C)CCC=C(C)C]

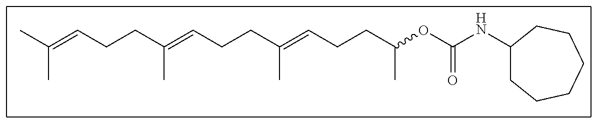 [SMILES: CC(C)=CCC/C(C)=C/CC/C(C)=C/CCC(C)OC(=O)NC1CCCCCC1]

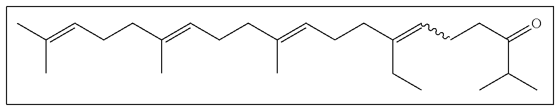 [SMILES: CCC(=CCCC(=O)C(C)C)CC/C=C(\C)CC/C=C(\C)CCC=C(C)C]

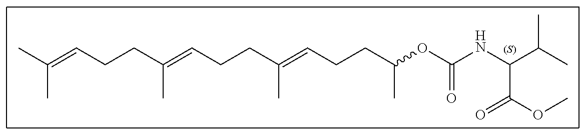

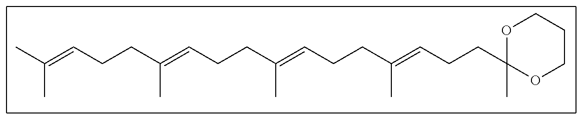 [SMILES: CC(C)=CCC/C(C)=C/CC/C(C)=C/CC/C(C)=C/CCC1(C)OCCCO1]

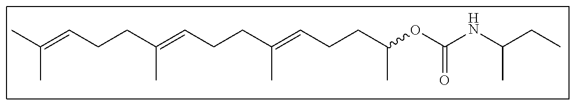 [SMILES: CCC(C)NC(=O)OC(C)CC/C=C(\C)CC/C=C(\C)CCC=C(C)C]

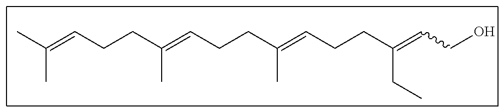 [SMILES: CCC(=CCO)CC/C=C(\C)CC/C=C(\C)CCC=C(C)C]

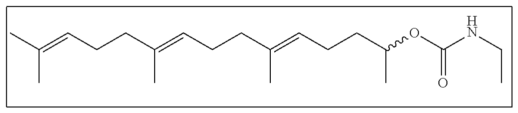 [SMILES: CCNC(=O)OC(C)CC/C=C(\C)CC/C=C(\C)CCC=C(C)C]

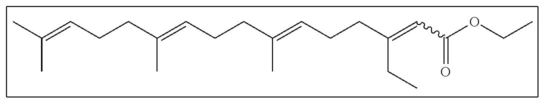 [SMILES: CCOC(=O)C=C(CC)CC/C=C(\C)CC/C=C(\C)CCC=C(C)C]

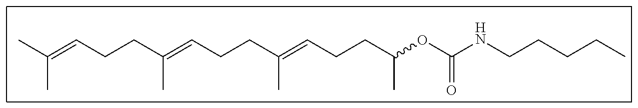 [SMILES: CCCCCNC(=O)OC(C)CC/C=C(\C)CC/C=C(\C)CCC=C(C)C]

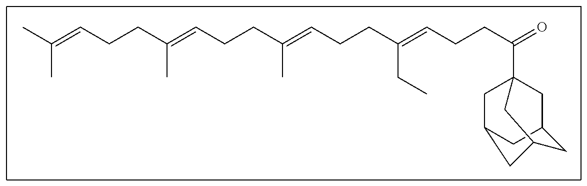 [SMILES: CC/C(=C\CCC(=O)C12CC3CC(CC(C3)C1)C2)CC/C=C(\C)CC/C=C(\C)CCC=C(C)C]

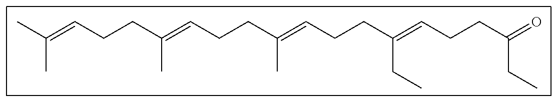 [SMILES: CCC(=O)CC/C=C(\CC)CC/C=C(\C)CC/C=C(\C)CCC=C(C)C]

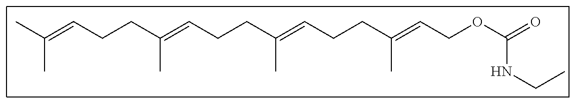 [SMILES: CCNC(=O)OC/C=C(\C)CC/C=C(\C)CC/C=C(\C)CCC=C(C)C]

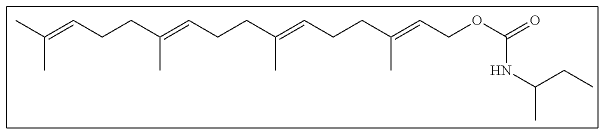 [SMILES: CCC(C)NC(=O)OC/C=C(\C)CC/C=C(\C)CC/C=C(\C)CCC=C(C)C]

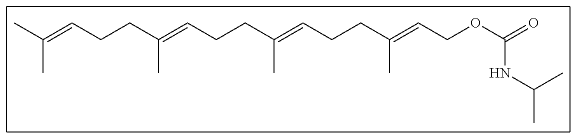 [SMILES: CC(C)=CCC/C(C)=C/CC/C(C)=C/CC/C(C)=C/COC(=O)NC(C)C]

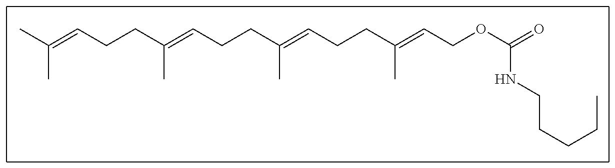 [SMILES: CCCCCNC(=O)OC/C=C(\C)CC/C=C(\C)CC/C=C(\C)CCC=C(C)C]

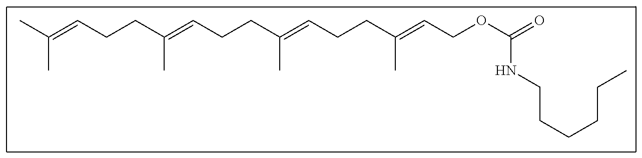 [SMILES: CCCCCCNC(=O)OC/C=C(\C)CC/C=C(\C)CC/C=C(\C)CCC=C(C)C]

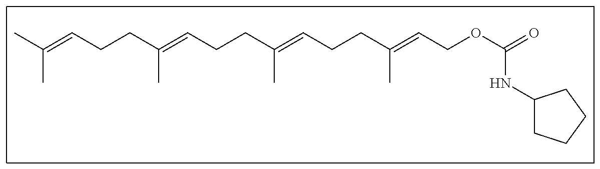 [SMILES: CC(C)=CCC/C(C)=C/CC/C(C)=C/CC/C(C)=C/COC(=O)NC1CCCC1]

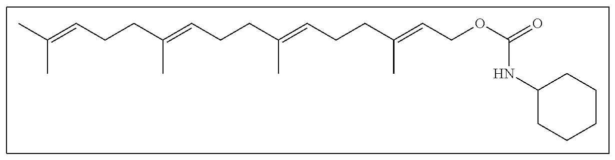 [SMILES: CC(C)=CCC/C(C)=C/CC/C(C)=C/CC/C(C)=C/COC(=O)NC1CCCCC1]

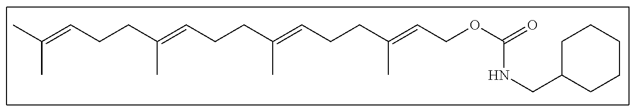 [SMILES: CC(C)=CCC/C(C)=C/CC/C(C)=C/CC/C(C)=C/COC(=O)NCC1CCCCC1]

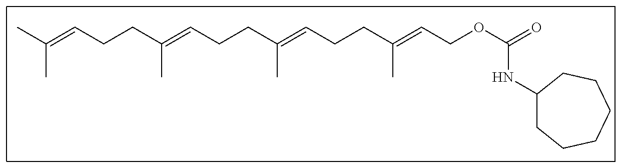 [SMILES: CC(C)=CCC/C(C)=C/CC/C(C)=C/CC/C(C)=C/COC(=O)NC1CCCCCC1]

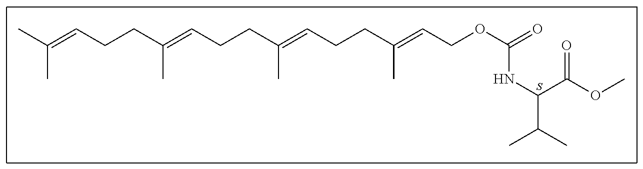

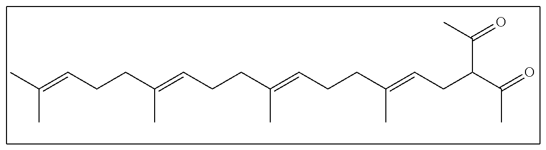 [SMILES: CC(=O)C(C/C=C(\C)CC/C=C(\C)CC/C=C(\C)CCC=C(C)C)C(C)=O]

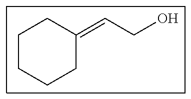

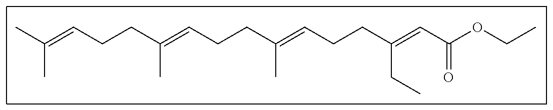 [SMILES: CCOC(=O)/C=C(\CC)CC/C=C(\C)CC/C=C(\C)CCC=C(C)C]

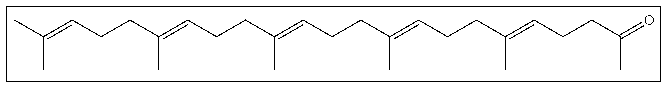 [SMILES: CC(=O)CC/C=C(\C)CC/C=C(\C)CC/C=C(\C)CC/C=C(\C)CCC=C(C)C]

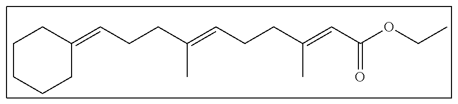 [SMILES: CCOC(=O)/C=C(\C)CC/C=C(\C)CCC=C1CCCCC1]

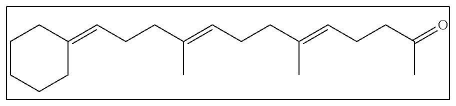 [SMILES: CC(=O)CC/C=C(\C)CC/C=C(\C)CCC=C1CCCCC1]

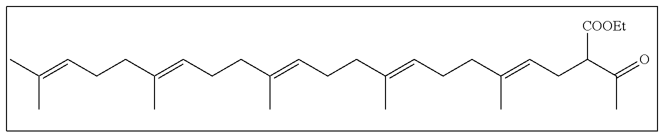 [SMILES: CCOC(=O)C(C/C=C(\C)CC/C=C(\C)CC/C=C(\C)CC/C=C(\C)CCC=C(C)C)C(C)=O]

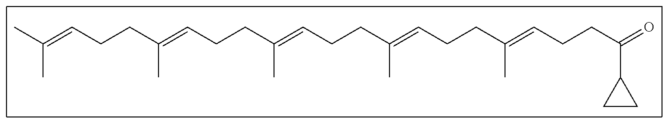 [SMILES: CC(C)=CCC/C(C)=C/CC/C(C)=C/CC/C(C)=C/CC/C(C)=C/CCC(=O)C1CC1]

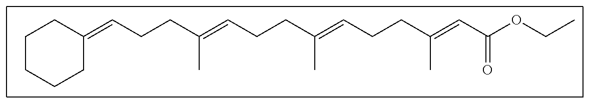 [SMILES: CCOC(=O)/C=C(\C)CC/C=C(\C)CC/C=C(\C)CCC=C1CCCCC1]

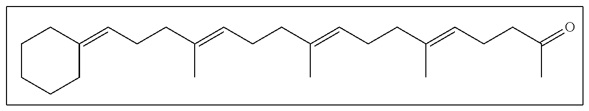 [SMILES: CC(=O)CC/C=C(\C)CC/C=C(\C)CC/C=C(\C)CCC=C1CCCCC1]

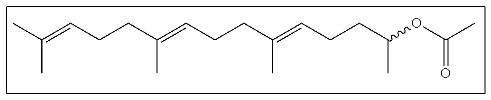 [SMILES: CC(=O)OC(C)CC/C=C(\C)CC/C=C(\C)CCC=C(C)C]

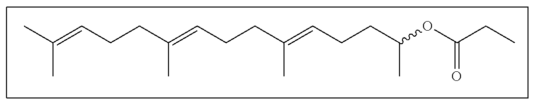 [SMILES: CCC(=O)OC(C)CC/C=C(\C)CC/C=C(\C)CCC=C(C)C]

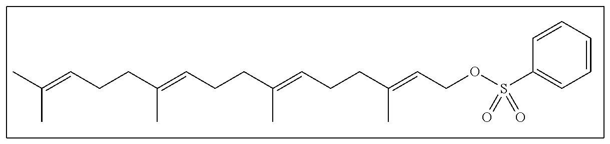 [SMILES: CC(C)=CCC/C(C)=C/CC/C(C)=C/CC/C(C)=C/COS(=O)(=O)c1ccccc1]

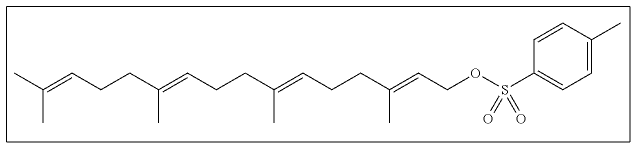 [SMILES: CC(C)=CCC/C(C)=C/CC/C(C)=C/CC/C(C)=C/COS(=O)(=O)c1ccc(C)cc1]

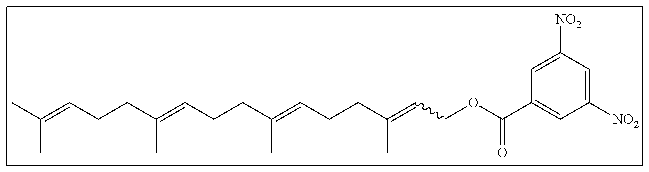 [SMILES: CC(C)=CCC/C(C)=C/CC/C(C)=C/CCC(C)=CCOC(=O)c1cc([N+](=O)[O-])cc([N+](=O)[O-])c1]

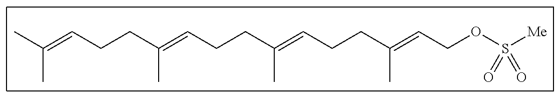 [SMILES: CC(C)=CCC/C(C)=C/CC/C(C)=C/CC/C(C)=C/COS(C)(=O)=O]

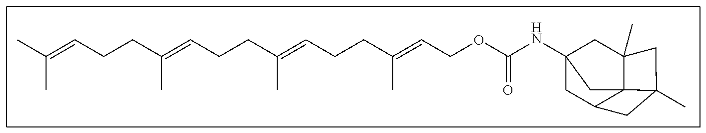 [SMILES: CC(C)=CCC/C(C)=C/CC/C(C)=C/CC/C(C)=C/COC(=O)NC12CC3CC4(C)CC(C)(C1)C34C2]

In one aspect, the GGA derivative is of Formula (IV):

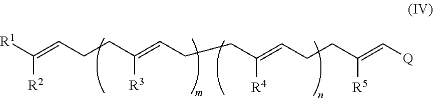

or a tautomer thereof, or a pharmaceutically acceptable salt of each thereof, wherein m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl, or R⁵ and Q together with the intervening carbon atoms form a 6 membered aryl ring, or a 5-8 membered cycloalkenyl ring, or a 5-14 membered heteroaryl or heterocycle, wherein each aryl, cycloalkenyl, heteroaryl, or heterocycle, ring is optionally substituted with 1-2 substituents selected from the group consisting of halo, hydroxy, oxo, —N(R¹⁰)₂, and C₁-C₆ alkyl group;
Q is a 6-10 membered aryl or a 5-14 membered heteroaryl or heterocycle containing up to 6 ring heteroatoms, wherein the heteroatom is selected from the group consisting of O, N, S, and oxidized forms of N and S, and further wherein the aryl, heteroaryl, or heterocyclyl ring is optionally substituted with 1-2 substituents selected from the group consisting of hydroxy, oxo, —N(R¹⁰)₂, and C₁-C₆ alkyl group, wherein the alkyl group is optionally substituted with 1-3 substituents selected from hydroxy, NH₂, —CO₂H or an ester or an amide thereof, a 5-9 membered heteroaryl containing up to 3 ring heteroatoms, wherein the heteroaryl is optionally substituted with 1-3 hydroxy, —N(R¹⁰)2, and C₁-C₆ alkyl group, and phenyl optionally substituted with 1-3 substituents selected from the group consisting of C₁-C₆ alkyl, hydroxy, and halo groups; and
wherein each R¹⁰ independently is hydrogen or C₁-C₆ alkyl.

As used herein, the compound of Formula (IV) includes tautomers and optical isomers such as enantiomers and diastereomers. As also used herein, an ester refers preferably to a phenyl or a C₁-C₆ alkyl ester, which phenyl or alkyl group is optionally substituted with a amino group. As used herein, an amide refers preferably to a moiety of formula —CON(R¹⁰)₂, wherein R¹⁰ is defined as above.

In some embodiment, Q is selected from a group consisting of oxazole, oxadiazole, oxazoline, azalactone, imidazole, diazole, triazole, and thiazole, wherein each heteroaryl or heterocycle is optionally substituted as disclosed above.

In one embodiment, the GGA derivative utilized is of formula:

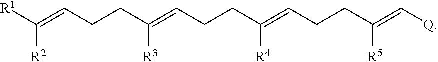

In another embodiment, the GGA derivative utilized is of formula:

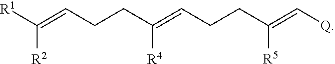

wherein R¹, R², R⁴, R⁵, and Q are defined as in any aspect and embodiment herein.

In another embodiment, Q is selected from the group consisting of:

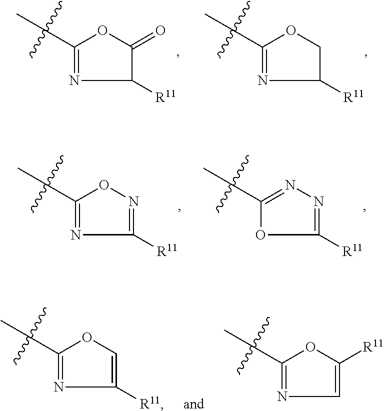

wherein R¹¹ is defined as above. In another embodiment, Q is phenyl, optionally substituted as described herein. In another embodiment, Q is benzimidazole, benzindazole, and such other 5-6 fused 9-membered bicyclic heteroaryl or heterocycle. In another embodiment, Q is quinoline, isoquinoline, and such other 6-6 fused 10 membered heteroaryl or heterocycle. In another embodiment, Q is benzodiazepine or a derivative thereof, such as, a benzodiazepinone. Various benzodiazepine and derivatives thereof are well known to the skilled artisan.

In another embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

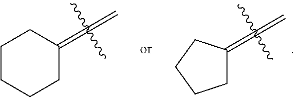 [SMILES: *=C1CCCCC1 ; *=C1CCCC1]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, this invention provides a compound selected from the group consisting of:

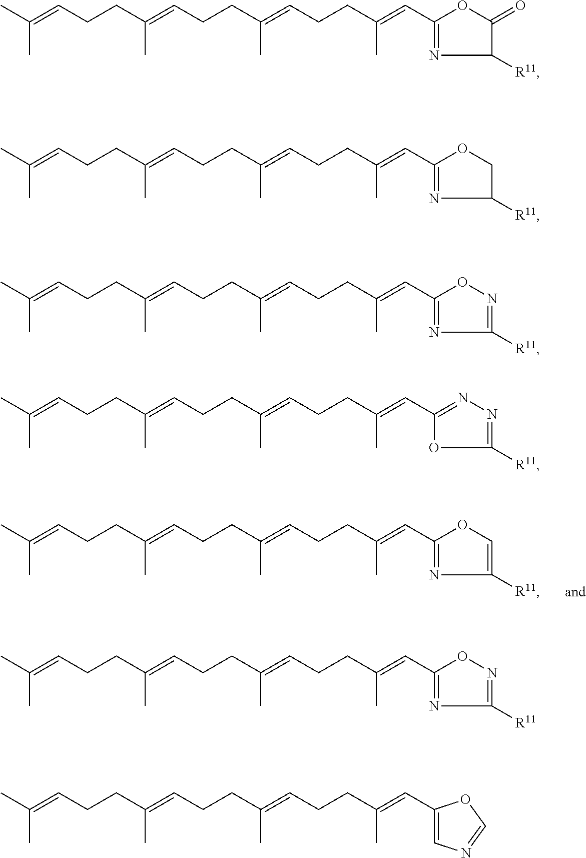

wherein R¹¹ is defined as above.
In another aspect, GGA derivatives utilized herein are of formula (V):

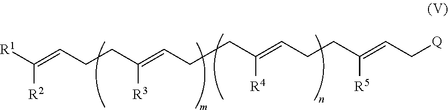

wherein
m is 0 or 1;
n is 0, 1, or 2;
each R¹ and R² independently are C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently is hydrogen or C₁-C₆ alkyl;
Q is selected from the group consisting of:

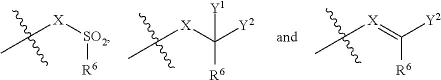

when X is bonded via a single bond, X is —O—, —NR⁷—, or —CR⁸R⁹—, and when X is bonded via a double bond, X is —CR⁸—;
Y¹ is hydrogen or —OR¹⁰;
Y² is —OR¹¹, —NHR¹², or —O—CO—NR¹³R¹⁴, or Y¹ and Y² are joined to form an oxo group (═O), an imine group (═NR¹⁵), a oxime group (═N—OR¹⁶), or a substituted or unsubstituted vinylidene (═CR¹⁸R¹⁹);
R⁶ is C₁-C₆ alkyl, C₁-C₆ alkyl substituted with 1-3 alkoxy or 1-5 halo groups, C₂-C₆ alkenyl, C₂-C₆ alkynyl, C₃-C₁₀ cycloalkyl, C₃-C₈ heterocyclyl, C₆-C₁₀ aryl, C₂-C₁₀ heteroaryl, or —NR²⁰R²¹, wherein each cycloalkyl or heterocyclyl is optionally substituted with 1-3 C₁-C₆ alkyl groups, and wherein each aryl or heteroaryl is optionally substituted independently with 1-3 nitro and C₁-C₆ alkyl groups;
R⁷ is hydrogen or together with R⁶ and the intervening atoms form a 5-7 membered ring optionally substituted with 1-3 C₁-C₆ alkyl groups;
each R⁸ and R⁹ independently are hydrogen, C₁-C₆ alkyl, —COR⁸¹, —CO₂R⁸¹, or —CONHR⁸², or R⁸ together with R⁶ and the intervening atoms form a 5-7 membered cycloalkyl or heterocyclyl ring optionally substituted with 1-3 C₁-C₆ alkyl groups;
R¹⁰ is C₁-C₆ alkyl;
each R¹¹ and R¹² independently are C₁-C₆ alkyl, C₃-C₁₀ cycloalkyl, —CO₂R¹⁷, or —CON(R¹⁷)₂;
R¹³ is:

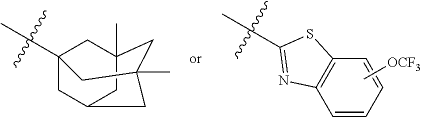

R¹⁴ is hydrogen or C₁-C₆ alkyl;
R¹⁵ is C₁-C₆ alkyl or C₃-C₁₀ cycloalkyl optionally substituted with 1-3 C₁-C₆ alkyl groups, or is:

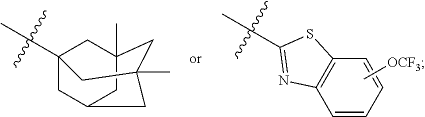

R¹⁶ is hydrogen, C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;
each R¹⁷ independently are hydrogen, C₃-C₁₀ cycloalkyl, C₁-C₆ alkyl optionally substituted with 1-3 substituents selected from the group consisting of —CO₂H or an ester thereof, aryl, C₃-C₈ heterocyclyl, or two R¹⁷ groups together with the nitrogen atom they are bonded to form a 5-7 membered heterocycle;
R¹⁸ is hydrogen or C₁-C₆ alkyl;
R¹⁹ is hydrogen, C₁-C₆ alkyl substituted with 1-3 hydroxy groups, —CHO, or is CO₂H or an ester thereof;
one or both of R²⁰ and R²¹ independently are hydrogen, C₁-C₆ alkyl, optionally substituted with —CO₂H or an ester thereof, C₃-C₁₀ cycloalkyl, C₃-C₈ heterocyclyl, C₂-C₁₀ aryl, or C₂-C₁₀ heteroaryl, or is C₃-C₁₀ cycloalkyl, C₃-C₈ heterocyclyl, C₆-C₁₀aryl, or C₂-C₁₀ heteroaryl, wherein each cycloalkyl, heterocyclyl, aryl, or heteroaryl is optionally substituted with 1-3 alkyl groups, or R²⁰ and R²¹ together with the nitrogen atom they are bonded to form a 5-7 membered heterocycle, and if only one of R²⁰ and R²¹ are defined as above, then the other one is

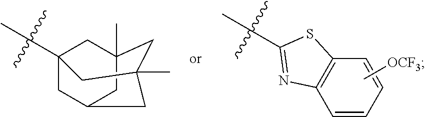

and
R⁶¹ is C₁-C₆ alkyl; and
R⁸² is:

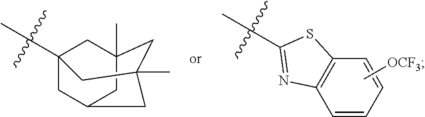

provided that, when X is bonded via a single bond, and R⁸ or R⁹ is not —CONHR⁸², Y¹ and Y² are joined to form an imine group (═NR¹⁵), and R¹⁵ is:

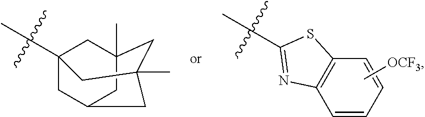

or Y² is —O—CO—NR¹³R¹⁴;
or provided that, when Q is:

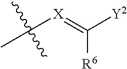

and R⁸ is not —CONHR⁸², Y² is —O—CO—NR¹³R¹⁴;
or provided that, when Q is —O—CO—NR²⁰R²¹, then at least one of R²⁰ and R²¹ is:

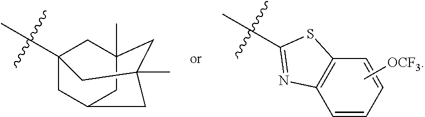

In one embodiment, the GGA derivative utilized are of formula:

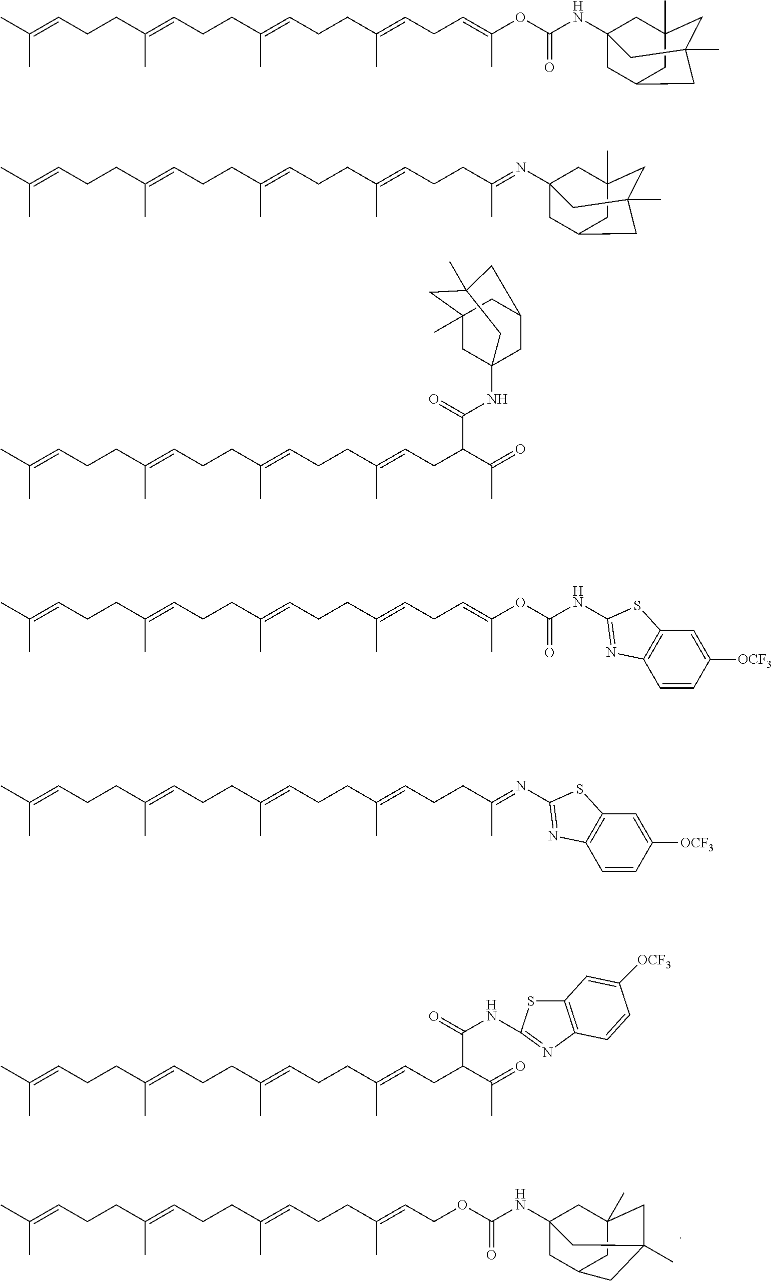

In certain aspects, the compound utilized is of Formula (XXI) or (XXII):

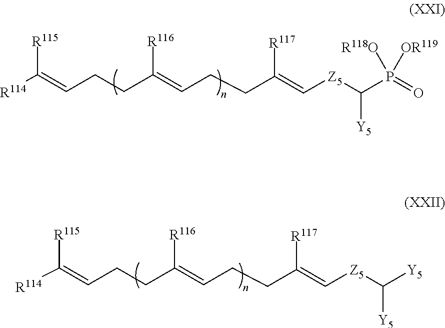

or a pharmaceutically acceptable salt thereof
wherein
R¹¹⁴ and R¹¹⁵ are independently selected from the group consisting of hydrogen, C₁-C₆ alkyl, C₃-C₇ cycloalkyl, C₂-C₆ alkenyl, C₁-C₆ alkynyl, optionally substituted C₆-C₂₀ aryl, optionally substituted C₆-C₂₀ aryl-C₁-C₆alkyl, optionally substituted heteroaryl and optionally substituted heteroaryl-C₁-C₆ alkyl, each heteroaryl having 2-14 ring carbon atoms and 1-6 ring heteroatoms selected preferably from N, O, S, and P, wherein each substituted aryl or substituted heteroaryl is independently substituted with 1-3 substituents selected from —OH, halo, C₁-C₆ alkyl, C₁-C₆ alkoxy, —NO₂, and —NR¹¹⁰R¹¹¹ groups; or R¹¹⁴ and R¹¹⁵ together with the carbon atom they are attached to form a C₃-C₇ cycloalkyl ring optionally substituted with 1-3 C₁-C₆ alkyl groups;
R¹¹⁶ and R¹¹⁷ independently are hydrogen or C₁-C₆ alkyl;
each R¹¹⁰ and R¹¹¹ is independently hydrogen, C₁-C₆ alkyl or C₆-C₂₀ aryl; or R¹¹⁰ and R¹¹¹ together with the nitrogen to which they are attached form a C₃-C₇ heterocycle;
wherein each aryl group of R¹¹⁰ and R¹¹¹ is optionally substituted with 1-3 C₁-C₆ alkyl, C₁-C₆ alkoxy, C₁-C₆ alkanoyl, C₁-C₆ alkanoyloxy, C₁-C₆ alkoxycarbonyl, halo, cyano, nitro, carboxy, trifluoromethyl, trifluoromethoxy, NR¹¹²R¹¹³ or S(O)₂NR¹¹²R¹¹³ groups, wherein each R¹¹² and R¹¹³ is independently hydrogen or C₁-C₆ alkyl;
each R¹¹⁸ and R¹¹⁹ are independently selected from the group consisting of a hydrogen, C₁-C₆ alkyl, and a group of Formula (XXIII):

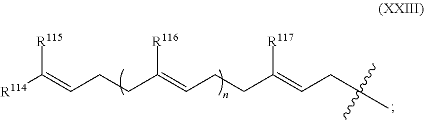

wherein R¹¹⁴-R¹¹⁷ and n are as defined as herein;
Y₅ is —P(═O)(OR¹¹⁸)(OR¹¹⁹), —CO₂R⁵²⁰ or —SO₂OR⁵²⁰, wherein R⁵²⁰ is selected from the group consisting of a hydrogen and C₁-C₆ alkyl;

### Z is

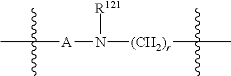

wherein R¹²¹ is hydrogen or C₁-C₆ alkyl; A is C₁-C₅ alkylene which may have a substituent selected from —OH, halo, C₁-C₆ alkyl, and C₁-C₆ alkoxy groups on each carbon;
r is 0, 1, 2, 3, 4 or 5; and

n is 0, 1, 2, 3, 4 or 5.

In one aspect, the GGA derivative utilized herein is of formula:

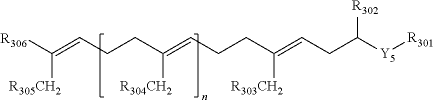

wherein R₃₀₁ is a lower (e.g. C₁-C₆) alkyl group, optionally substituted with 1 to 4 substituents selected from the group consisting of halogen, hydroxyl; lower alkyl; lower alkoxy; halogenated lower alkyl; halogenated lower alkoxy; cyano; a 5- or 6-membered (hetero) aromatic ring which may be substituted by hydroxyl, lower alkyl, lower alkoxy, halogen, amino, lower alkylamino; cyano, nitro; and other (substituted) (hetero) aromatic rings;
R₃₀₂ is hydrogen or C₁-C₄ alkyl; Both the R and S configurations are encompassed.
R₃₀₃, R₃₀₄ and R₃₀₅ are independently selected from hydrogen, substituted and nonsubstituted C₁-C₄ alkyl groups
R₃₀₆ is CH(O) or CₘH₂ₘ—X₆, wherein m is 1-3 and X₆ is —H, —OH or a 5- or 6-membered (hetero)aromatic ring; and
Y₅ is —C(O)— or —C(═NOR₇)— wherein R₃₀₇ is hydrogen or a C₁-C₄ alkyl group.

In another aspect, the GGA derivative utilized is of formula:

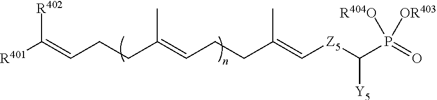

wherein R⁴⁰¹ and R⁴⁰² each represent a hydrogen atom, a lower alkyl, cycloalkyl, alkenyl or alkynyl group, an aryl group which may be substituted, an arylalkyl group in which the aryl group may be substituted, or a heteroaryl or heteroarylalkyl group: R⁴⁰³ and R⁴⁰⁴ each represent a hydrogen atom, a lower alkyl group or an alkali metal; Y₅ represents a group represented by the formula:

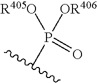

wherein R⁴⁰⁵ and R⁴⁰⁶ each represent a hydrogen atom, a lower alkyl group or an alkali metal or a group represented by the formula: —CO₂R⁴⁰⁷ (wherein R⁴⁰⁷ represents a hydrogen atom, a lower alkyl group or an alkali metal); Z₅ represents a group represented by the Formula: —(CH₂)₂— (wherein m is an integer of 0 to 3), a group represented by the formula: —(CH₂)ₚ—CH═CH—(CH₂)_{q}— (wherein p is 0 or 1 and q is 1 or 2) or a group represented by the Formula:

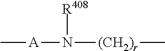

wherein R⁴⁰⁸ represents a hydrogen atom or a lower alkyl group; A represents an alkylene chain which has 1 to 5 carbon atoms and which may have a substituent on each carbon atom; and r is zero or an integer of 1 to 5); and n is zero or an integer of 1 to 5.

In some embodiments, the compounds separated herein include:

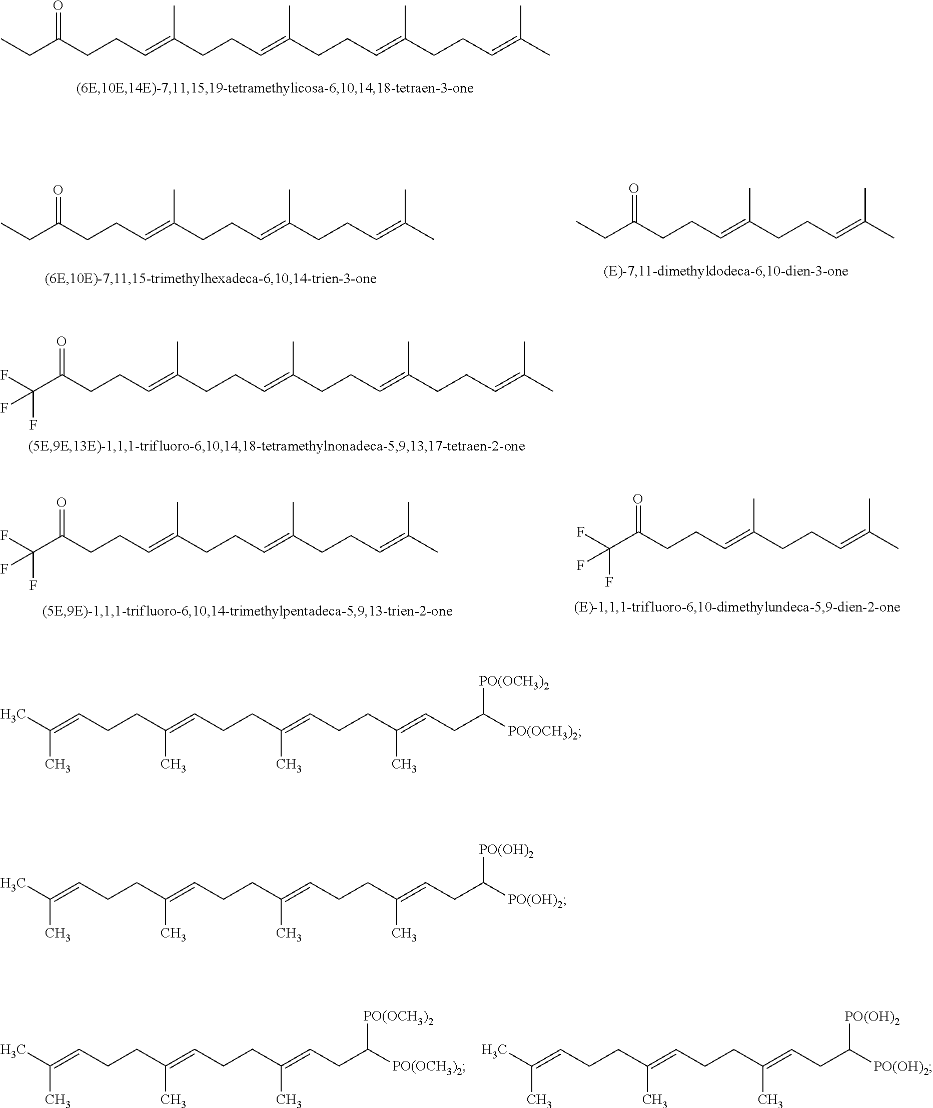

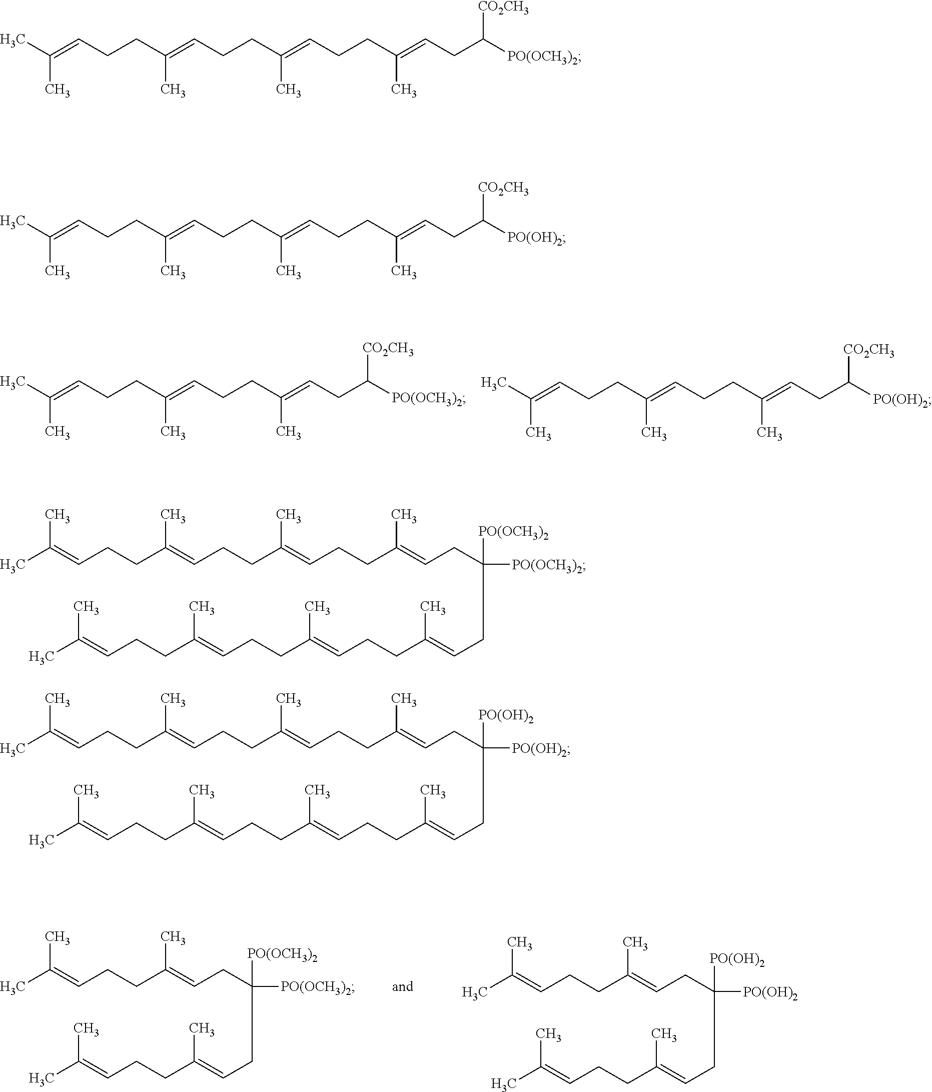

and the corresponding ethyl and other C₁-C₆ alkyl esters, and corresponding cis isomers.

In this disclosure, if a variable such as R¹ is used to denote more than one functionality, based on the formulas that variable refers to, the skilled artisan will readily appreciate the intended definition of that variable.

### Synthesis of GGA and GGA Derivatives

In various aspects, provided herein are processes for preparing compounds of formula (XXXI) and derivatives thereof, and intermediates used in their synthesis.

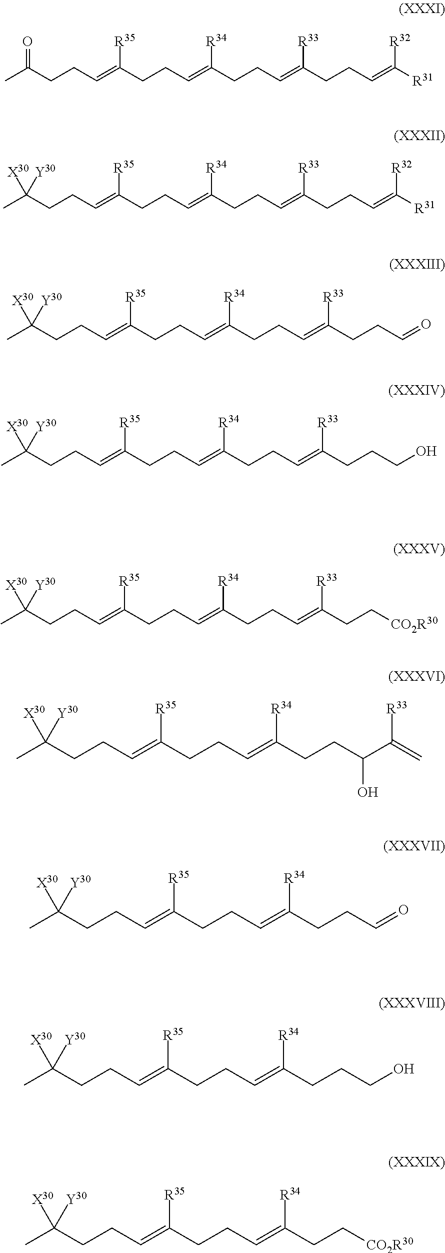

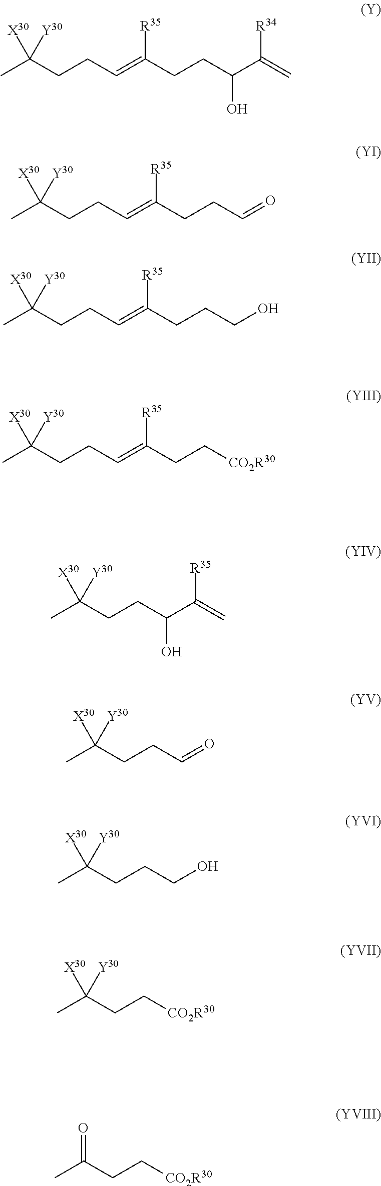

or a salt thereof, wherein the variables in the structures (XXXI)-(YVIII) are defined as in formula (II) above.

In one embodiment, X³⁰ and Y³⁰ together with the carbon atom they are attached to form a ring of formula:

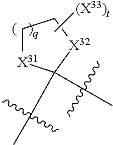

wherein each X³¹ and X³² are independently O, or S; q is 1 or 2; each X³³ is independently C₁-C₆ alkyl; and t is 0, 1, 2, or 3.

In one aspect, the GGA derivative has the formula YIX:

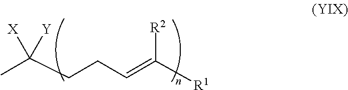

or a tautomer or pharmaceutically acceptable salt thereof, wherein X, Y, R³¹ and R³² are as defined herein; and
n is 1, 2, 3, 4 or 5.

In another aspect, the GGA derivative has the formula YX:

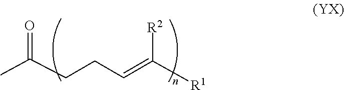

or a tautomer or pharmaceutically acceptable salt thereof, wherein R³¹, and n are as defined herein.

In another aspect, the GGA derivative has the formulas (YXI)-(YXV):

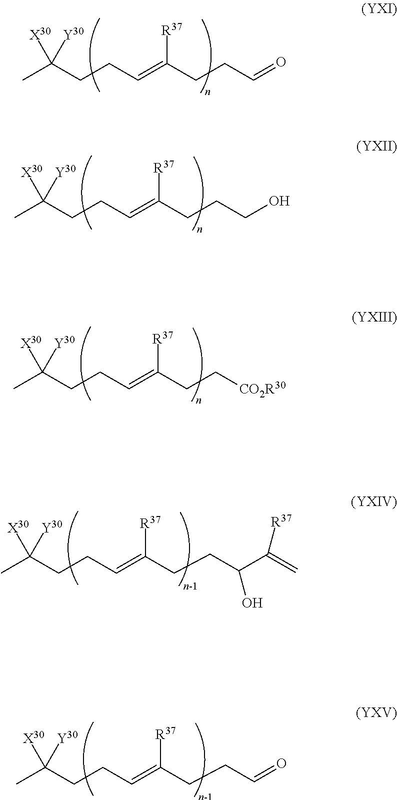

or a tautomer or pharmaceutically acceptable salt thereof, wherein:
X³⁰ and Y³⁰ are each independently OR³⁶, SR³⁶, or X³⁰ and Y³⁰ together with the carbon atom they are attached to form a 5-7 membered heterocyclic ring having 2 oxygen and/or sulfur atoms and optionally substituted with 1-3 C₁-C₆ alkyl groups,
each of R³⁶ is independently C₁-C₆ alkyl,
each of R³⁷ is independently H or C₁-C₆ alkyl and
n is an integer from 1 to 5.

In one embodiment, X³⁰ and Y³⁰ together with the carbon atom they are attached to form a cyclic ketal with two oxygen, two sulfur or one oxygen and one sulfur atom.

In one embodiment, X³⁰ and Y³⁰ together with the carbon atom they are attached to form a dioxolane, oxathiolane, dithiolane, dioxane, oxathiane or a dithiane ring.

This invention provides processes for the syntheses of GGA derivatives, cis-trans isomers, and sub-formulas thereof.

In one aspect, a process for preparing the GGA derivative of formula (XXXI) is provided. The process comprises contacting a compound of formula (XXXII) with an acid catalyst under conditions suitable for hydrolysis of compound of formula (XXXII) to produce a compound of formula (XXXI).

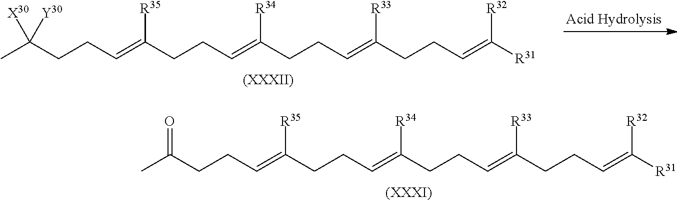

In one embodiment, the conditions suitable for hydrolysis of compound of formula (XXXII) to produce a compound of formula (XXXI) include contacting the compound of formula (XXXII) with an acid catalyst in an inert solvent at a suitable temperature. In one embodiment, the acid catalyst utilized in the process is selected from an aqueous acetic acid, formic acid, trifluoroacetic acid, sulfuric acid, hydrochloric acid, methanesulfonic acid, alkyl or aralkylsulfonic acid or a Lewis acid. The acid is preferably used in catalytic amount.

In another aspect, a process for preparing the GGA derivative of formula (XXXII) is provided. The process comprises subjecting a compound of formula (XXXIII) to an olefination reaction under conditions suitable for of compound of formula (XXXIII) to produce a compound of formula (XXXII).

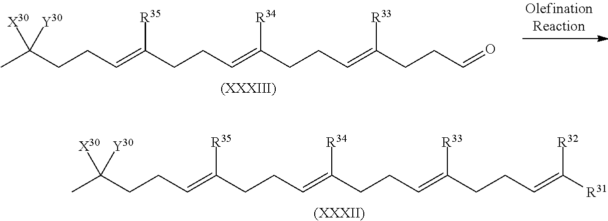

In one embodiment, the olefination is conducted using Wittig reaction. Wittig reaction or Wittig olefination refers to the reaction of a carbonyl compound, e.g. an aldehyde or a ketone, with a phosphonium ylide to an alkene. Typical Wittig reaction includes deprotonating a phosphonium salt by a base to form a phosphorane and reacting it with the aldehyde. The phosphonium salts or Wittig reagents utilized in the reaction can be obtained by reacting a phosphine, e.g., triphenylphosphine, with a primary or secondary halide under heated conditions, in the presence or absence of a solvent.

In one embodiment, the conditions suitable for olefination of compound of formula (XXXI) to produce a compound of formula (XXXII) include, for example, reacting the aldehyde of formula (III) with a phosphorane in a suitable solvent in the presence of a base. In one embodiment, the olefination of compound of formula (XXXIII) includes contacting compound (XXXIII) with a Wittig reagent, e.g., C(R³¹R³²)═P(R^{z})₃ wherein R^{z} is e.g., triphenyl group. In some embodiments, a composition comprising the compound of formula (XXXIII) with a Wittig reagent e.g., C(R³¹R³²)═PR^{z}, is provided. Suitable solvents include aliphatic or aromatic hydrocarbons, such as e.g., hexane, benzene or toluene, and ethers such as for example diethyl ether and tetrahydrofuran, or amides, such as e.g., dimethylformamide or hexamethylphosphoric acid triamide. In some cases alcohols or dimethyl sulphoxide can be used as solvent. Suitable bases for the Wittig reaction include metal alcoholates, such as for example sodium ethanolate, metal hydrides, such as for example sodium hydride, metal amides, such as e.g., sodium amide and organometalic compounds, such as for example phenyllithium or butyllithium. The vinyl group in the compound of formula (XXXII) can be formed with specificity and positional selectivity.

In one embodiment, the olefination is conducted using Wittig-Horner reaction. In other embodiments, the olefination is conducted using Peterson olefination reaction. Conditions suitable for these reactions will be apparent to one skilled in the art. For example, the Witting-Horner reaction can be conducted utilizing the Wittig reagent, as described for the Wittig reaction, but with lithium bases, such as e.g., n-butyl lithium, and at low temperatures. In Peterson olefination, for example, α-silylated carbanion is added to the carbonyl compound of formula (XXXIII) to give rise to two diastereomeric β-hydroxysilanes, which can be isolated and separately transformed further to alkenes.

In yet another aspect, a process for preparing the GGA derivative of formula (XXXIII) is provided. The process comprises oxidizing a compound of formula (XXXIV) under suitable conditions to produce a compound of formula (XXXIII).

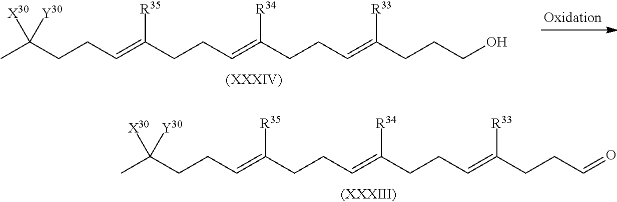

In another aspect, a process for preparing the GGA derivative of formula (XXXVII) is provided. The process comprises oxidizing a compound of formula (XXXVIII) under suitable conditions to produce a compound of formula (XXXVII).

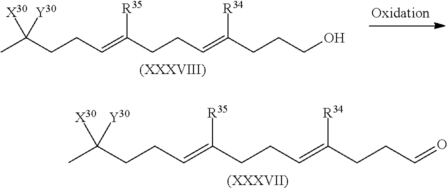

In another aspect, a process for preparing the GGA derivative of formula (YI) is provided. The process comprises oxidizing a compound of formula (YII) under suitable conditions to produce a compound of formula (YI).

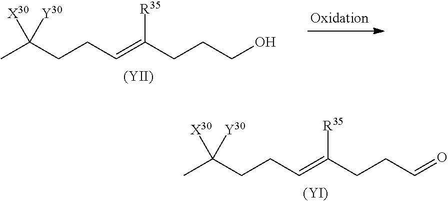

In another aspect, a process for preparing the GGA derivative of formula (XV) is provided. The process comprises oxidizing a compound of formula (YVI) under suitable conditions to produce a compound of formula (YV).

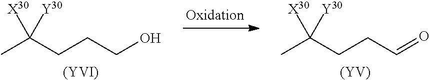

In some embodiments, conditions suitable for oxidation of compound of formula (XXXIV), (XXXVIII), (YII), (YVI) include, subjecting the compound to Moffatt oxidation. As will be appreciated by one skilled in the art, Moffat oxidation is the reaction of primary and secondary alcohols by dimethyl sulfoxide (DMSO) activated with a carbodiimide, such as dicyclohexylcarbodiimide (DCC) in presence of an acid to produce an alkoxysulfonium ylide which rearranges to generate aldehydes and ketones, respectively (K. E. Pfitzner and J. G. Moffatt, J. Am. Chem. Soc., 85, 3027 (1963)). Swern Oxidation may also be used, which in some embodiments employs DMSO and oxalyl chloride, at low temperatures, as is well known to the skilled artisan.

In some embodiments, other methods suitable for oxidation of an alcohol to an aldehyde can be utilized. For example, the alcohol can be oxidized under Parikh-Doering oxidation conditions using DMSO as the oxidant, activated by the sulfur trioxide pyridine complex in the presence of alkylamine base, e.g., triethylamine. In other embodiments, the alcohol compound can be oxidized under Swern oxidation conditions using oxalyl chloride, dimethyl sulfoxide (DMSO) and an organic base, such an alkylamine base, e.g., triethylamine.

In another aspect, a process for preparing the GGA derivative of formula (XXXIV) is provided. The process comprises reducing a compound of formula (XXXV) under suitable conditions to produce a compound of formula (XXXIV).

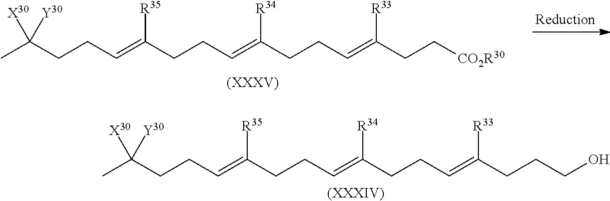

In another aspect, a process for preparing the GGA derivative of formula (XXXVIII) is provided. The process comprises oxidizing a compound of formula (XXXIX) under suitable conditions to produce a compound of formula (XXXVIII).

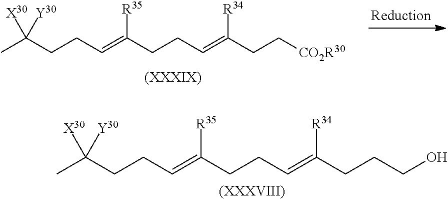

In another aspect, a process for preparing the GGA derivative of formula (XII) is provided. The process comprises oxidizing a compound of formula (XIII) under suitable conditions to produce a compound of formula (XII).

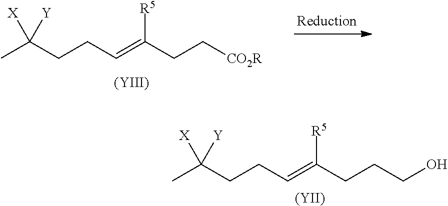

In another aspect, a process for preparing the GGA derivative of formula (YVI) is provided. The process comprises oxidizing a compound of formula (YVII) under suitable conditions to produce a compound of formula (YVI).

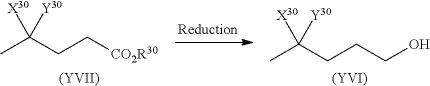

As will be appreciated by one skilled in the art, suitable reducing agents for the reduction of acid of formula (XXXV), (XXXIX) or (YIII) or (YVII) include reducing hydrides, preferably aluminum hydrides or borohydrides, more preferably metal aluminum hydrides in which the metal is a group I or group II metal such as lithium, sodium, potassium, calcium, magnesium or the like. Particularly preferred metal aluminum hydrides include lithium aluminum hydride (LAH), sodium aluminum hydride, and mixture thereof. The reduction is typically conducted in aprotic solvents such as ethers, e.g. tetrahydrofuran or aromatic hydrocarbons e.g., benzene and toluene, at low to reflux temperature using from about 0.5 to about 3.0 moles of hydride reducing agent per mole of compound of formula (XXXV). In one embodiment, the preferred reducing agent is lithium aluminum hydride. Suitable solvents include dioxane, toluene, diethyl ether, tetrahydrofuran (THF), dipropyl ether and the like. In one embodiment, the preferred solvent is diethyl ether or THF.

In another aspect, a process for preparing the GGA derivative of formula (XXXV) is provided. The process comprises reacting a compound of formula (XXXVI) under suitable conditions to produce a compound of formula (XXXV).

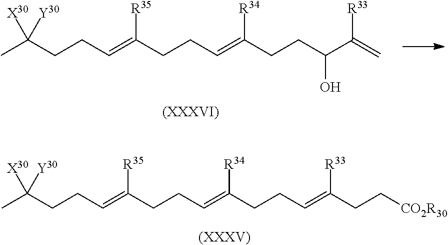

In another aspect, a process for preparing the GGA derivative of formula (XXXIX) is provided. The process comprises reacting a compound of formula (Y) under suitable conditions to produce a compound of formula (IX).

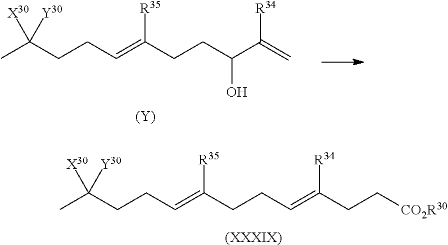

In another aspect, a process for preparing the GGA derivative of formula (YIII) is provided. The process comprises reacting a compound of formula (YIV) under suitable conditions to produce a compound of formula (YIII).

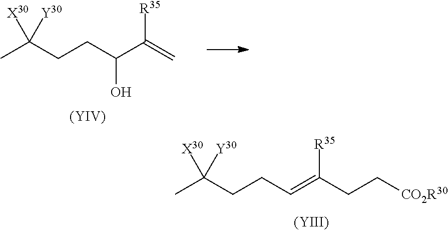

In some embodiments, suitable conditions include subjecting the allylic alcohol of formula (XXXVI), (Y) or (YIV) to Johnson-Claisen rearrangement to give the γ,δ-unsaturated ester, such as of formula (XXXV). In one embodiment, the compound of formula (XXXVI) is condensed with a tri-(C₁-C₆)alkyl orthoacetate and further the intermediate allyl-enol ether is rearranged, without isolation, in the presence of an acid in a reaction inert solvent. The orthoacetate utilized for the process is preferably selected from trimethyl orthoacetate and triethyl orthoacetate. In one embodiment, the orthoacetetate has the formula CH₃C(OR³⁰)₃, where R³⁰ is C₁-C₆ alkyl. In some embodiments, the acid is a weak acid, preferably a simple carboxylic acid such as a propionic acid or isobutyric acid, or an alkane or arene sulphonic acid e.g., p-toluene sulphonic acid. The process is carried out at an elevated temperature preferably the reflux temperature, under conditions where alcohol generated by the process can be removed from the reaction mixture.

In another aspect, a process for preparing the GGA derivative of formula (XXXVI) is provided. The process comprises alkenylating a compound of formula (XXXVII) with R³³C(—)═CH₂ under suitable conditions to produce a compound of formula (XXXVI).

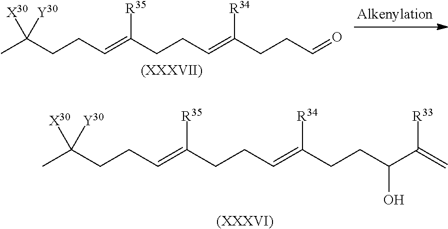

In another aspect, a process for preparing the GGA derivative of formula (Y) is provided. The process comprises alkenylating a compound of formula (YI) with R³⁴C(—)═CH₂ under suitable conditions to produce a compound of formula (Y).

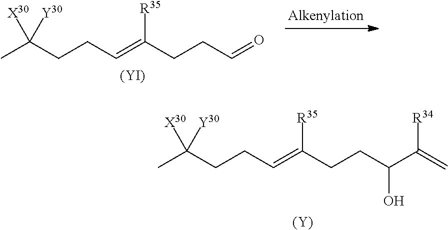

In another aspect, a process for preparing the GGA derivative of formula (YIV) is provided. The process comprises alkenylating a compound of formula (YV) with R³⁵C(—)═CH₂ under suitable conditions to produce a compound of formula (YIV).

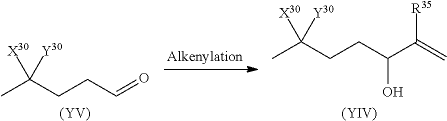

R³³, R³⁴, and R³⁵ are as defined herein above. In some embodiments, conditions suitable for the alkenylation of the aldehyde of formulas (XXXVII), (YI), and (YV) include, contacting the compound of formula (XXXVII) with an appropriate organometallic reagent in a reaction inert solvent. The organometallic reagent utilized for the C-alkenylation of carbonyl compounds to the allyl alcohol preferably contain magnesium halides or lithium moieties. Suitable inert solvents utilized in the process will be apparent to one skilled in the art. In one embodiment, the solvent is THF or diethyl ether.

In another aspect, a process for preparing the GGA derivative of formula (YVII) is provided. The process comprises reacting a compound of formula (YVIII) under suitable conditions to produce a compound of formula (YVII).

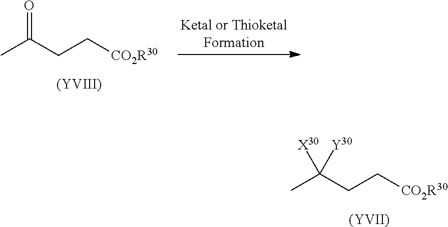

In some embodiments, conditions suitable for ketal, thioketal, or an oxa-thioketal (having an —O—C—S— moiety) formation include, reacting the carbonyl compound of formula (YVIII) with a suitable alcohol, alpha omega diol, a thiol, an alpha omega dithiol, or an omega hydroxy thiol solvent under acidic conditions. In one embodiment, the ester of Formula (YVIII) is reacted with a suitable alcohol such as e.g., ethylene glycol, mercaptoethanol and 1,2-dithioethanol in the presence of a suitable acid catalyst followed by azeotropic removal of water. Suitable acid catalysts include, e.g., strong mineral acids, such as sulfuric, hydrochloric, hydrofluoroboric, hydrobromic acids, p-toluenesulfonic acid, camphorosulfonic acid, methanesulfonic acid, and like. Various resins that contain protonated sulfonic acid groups are also useful as they can be easily recovered after completion of the reaction. Examples of acids also include Lewis acids. For example, boron trifluoride and various complexes of BF₃, such as e.g., BF₃ diethyl etherate. Silica, acidic-alumina, titania, zirconia, various acidic clays, and mixed aluminum or magnesium oxides can be used. Activated carbon derivatives comprising mineral acid, sulfonic acid, or Lewis acid derivatives can also be used.

In one aspect, a process for preparing the GGA derivative of formula (YX) is provided. The process comprises contacting a compound of formula (YIX) with an acid catalyst under conditions suitable for hydrolysis of compound of formula (YX) to produce a compound of formula (YIX).

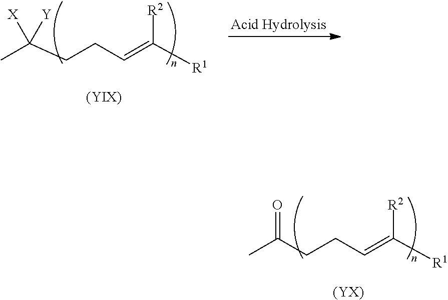

In one embodiment, the conditions suitable for hydrolysis of compound of formula (XXXII) to produce a compound of formula (XXXI) include contacting the compound of formula (XXXII) with an acid catalyst in a compatible solvent at a suitable temperature.

In one embodiment, the acid catalyst utilized in the process is selected from an aqueous acetic acid, formic acid, trifluoroacetic acid, sulfuric acid, hydrochloric acid, methanesulfonic acid, alkyl or aralkylsulfonic acid or a Lewis acid.

In one embodiment, the GGA prepared according to this invention is 5-trans GGA or substantially pure 5-trans GGA which is optionally free of cis GGA or is essentially free of cis GGA. In other embodiment, the GGA prepared according to this invention is 5-cis GGA or substantially pure 5-cis GGA which is optionally free of trans GGA or is essentially free of trans GGA.

The starting materials for the reactions described herein are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1 15 (John Wiley and Sons, 1991), Rodd's Chemistry of Carbon Compounds, Volumes 1 5 and Supplementals (Elsevier Science Publishers, 1989), Organic Reactions, Volumes 1 40 (John Wiley and Sons, 1991), March's Advanced Organic Chemistry, (John Wiley and Sons, 4^{th} Edition), and Larock's Comprehensive Organic Transformations (VCH Publishers Inc., 1989).

Levulinic esters, such as methyl levulinate or ethyl levulinate, can be converted to the corresponding ketal (X═Y═O), hemi-thioketal (X═O, Y═S), or dithio-ketal, (X═Y═S), by reacting with ethylene glycol, mercapto ethanol, or ethane-1,2-dithiol, under acidic conditions that facilitate removal of water. Typical acid catalysts include p-toluenesulfonic acid, or acetic acid and boron trifluoride-etherate. Solvents used for such transformations include benzene, toluene and methylene chloride. Water is removed by azeotropic distillation or by reaction with an ortho-ester such as triethyl ortho-formate or triethyl ortho-acetate. (Reaction 1)

Conversion of the ketal-ester, from Reaction 1 to the corresponding aldehyde, can be accomplished in a single step shown in Reaction 2 by reduction with a hindered active metal hydride such as di-isobutyl aluminum hydride at reduced temperatures in ether, followed by quenching with ethyl acetate to consume excess reagent. Temperatures for such reactions typically must be kept below −35° C. to minimize over-reduction to the alcohol. (Reaction 2)

Alternatively, the aldehyde, can be prepared in higher yield and greater purity in two separate steps. The first step, Reaction 3, involves complete reduction of the ester to the corresponding alcohol, with a strong reducing agent such as lithium aluminum hydride in diethyl ether or THF. This reduction is followed by oxidation of the alcohol to the aldehyde per Reaction 4, by one of the several methods listed below. (Reaction 4)

Use of chromium trioxide in pyridine for oxidation of alcohols to aldehydes is reported. Alternatively, this oxidation can be accomplished with dimethyl sulfoxide and any of a variety of dehydrating agents. Published examples include various acid chlorides, acid anhydrides, and carbodiimides.

These reactions typically require temperatures below −35° C. prevent side reactions. The method employing a sulfur trioxide-pyridine complex in the presence of triethylamine can be conducted at room temperature with minimal side reactions. (Reaction 5)

The aldehyde is reacted with 2-propenyl lithium or its Grignard equivalent, to give an allylic alcohol. This alcohol can be converted to olefinic esters in high yield with high stereoselectivity. (Reaction 6)

The product is then subjected to the transformations of Reactions 3 through 6 for two additional cycles to yield an ester. This ester is then reduced and oxidized as in Reactions 3 and 4 to give the alcohol in (Reaction 7) and the aldehyde in (Reaction 8).

The terminal olefin can be added by a Wittig reaction using propylidene triphenylphosphosphineylide, generated from the commercially available isopropyl(triphenyl)phosphonium bromide as shown in Reaction 9.

The final product is produced by hydrolysis of the ketal, hemithioketal, or the dithioketal.

Other methods for making GGA or certain GGA derivatives utilized herein are described in PCT publication no. WO 2012/031028 and PCT application no. PCT/US2012/027147, each of which is incorporated herein by reference in its entirety. Other GGA derivatives can be prepared by appropriate substitution of reagents and starting materials, as will be well known to the skilled artisan upon reading this disclosure.

The reactions are preferably carried out in a suitable inert solvent that will be apparent to the skilled artisan upon reading this disclosure, for a sufficient period of time to ensure substantial completion of the reaction as observed by thin layer chromatography, ¹H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure.

The compounds utilized in this invention are synthesized, e.g., from a compound of formula (III-A):

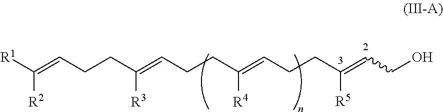

wherein n, R¹-R⁵ and [symbol] are defined as in Formula (I) above, following various well known methods upon substitution of reactants and/or altering reaction conditions as will be apparent to the skilled artisan upon reading this disclosure. The compound of Formula (III-A) is itself prepared by methods well known to a skilled artisan, for example, and without limitation, those described in PCT Pat. App. Pub. No. WO 2012/031028 and PCT Pat. App. No. PCT/US2012/027147 (each supra). An illustrative and non-limiting method for synthesizing a compound of Formula (III-A), where n is 1, is schematically shown below.

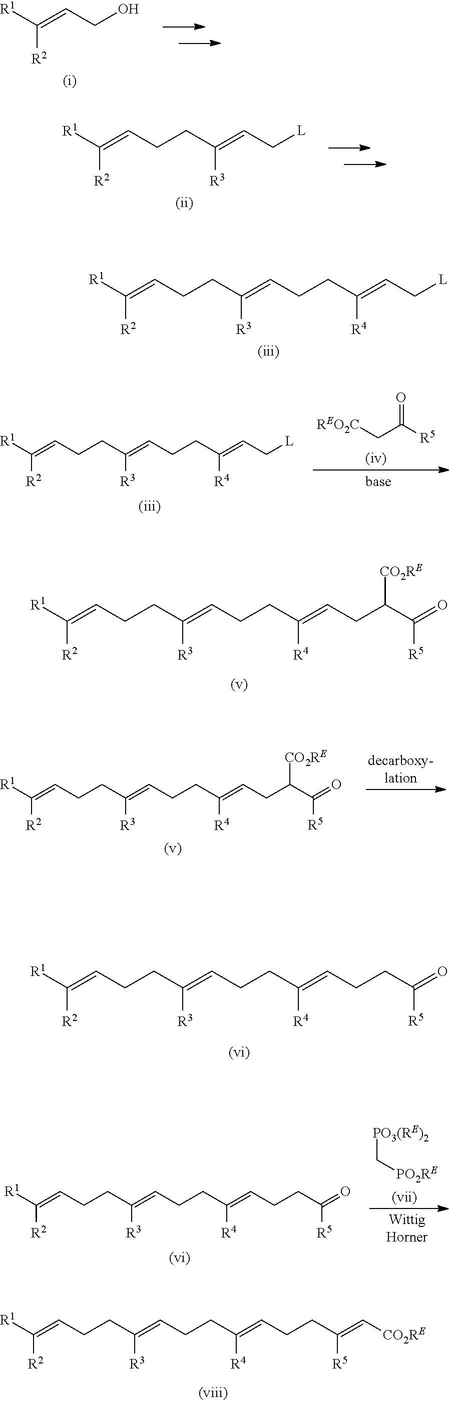

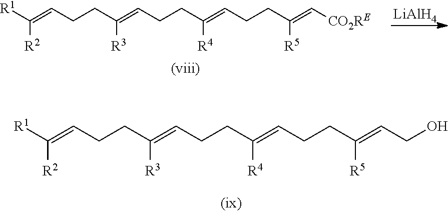

Starting compound (iii), which is synthesized from compound (i) by adding isoprene derivatives as described here, is alkylated with a beta keto ester (iv), in the presence of a base such as an alkoxide, to provide the corresponding beta-ketoester (v). Compound (v) upon alkaline hydrolysis followed by decarboxylation provides ketone (vi). Keto compound (vi) is converted, following a Wittig Horner reaction with compound (vii), to the conjugated ester (viii). Compound (viii) is reduced, for example with LiAlH₄, to provide alcohol (ix).

As will be apparent to the skilled artisan, a compound of Formula (III), where n is 2, is synthesized by repeating the reaction sequence of alkylation with a beta-keto ester, hydrolysis, decarboxylation, Wittig-Horner olefination, and LiAlH₄ reduction.

Certain illustrative and non-limiting synthesis of compounds utilized in this invention are schematically shown below. Compounds where Q¹ is —(C═S)— or —SO₂— are synthesized by substituting the carbonyl group of the reactants employed, as will be apparent to the skilled artisan.

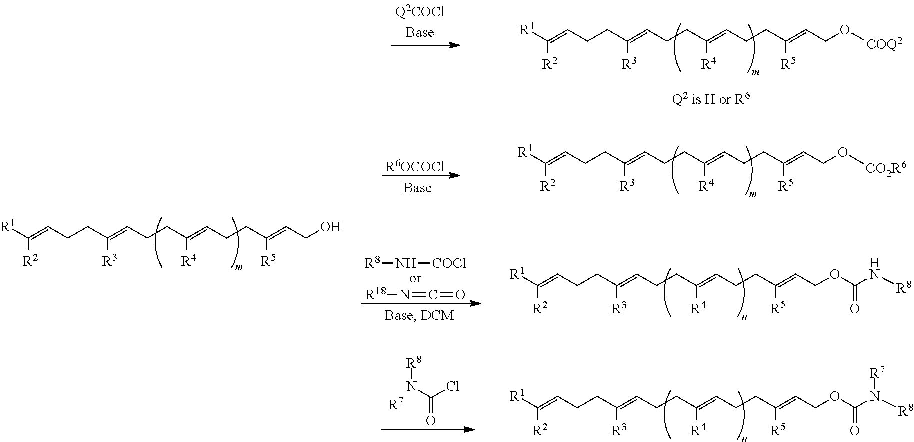

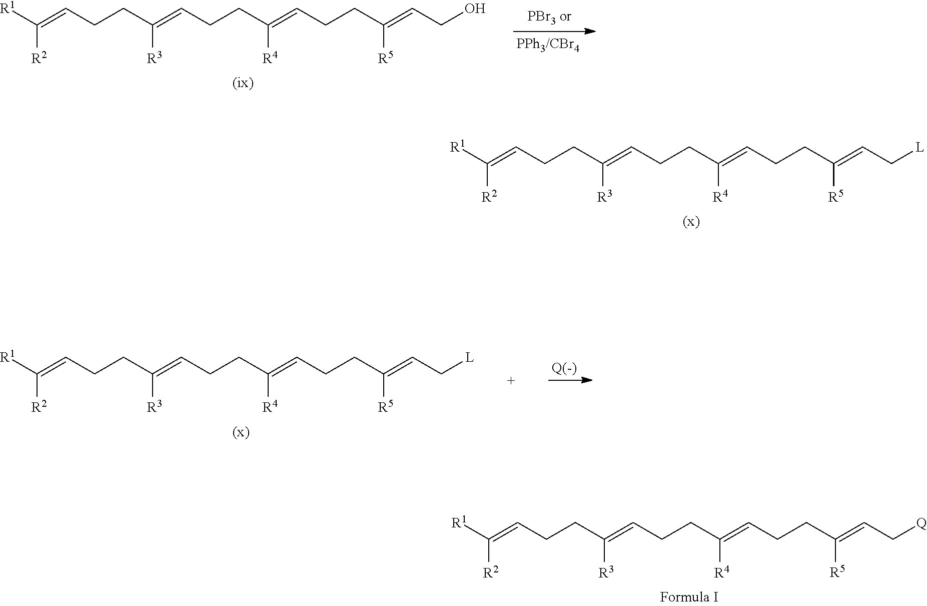

As shown above, R^{E} is alkyl.

Compound (ix) with alcohol functionality is an intermediate useful for preparing the compounds utilized in this invention. Compound (x), where L is an R^{s}SO₂— group is made by reacting compound (ix) with R^{s}SO₂Cl in the presence of a base. The transformation of compound (iii) to compound (x) illustrates methods of adding isoprene derivatives to a compound, which methods are suitable to make compound (iii) from compound (i). Intermediate (ix) containing various R¹-R⁵ substituents are prepared according to this scheme as exemplified herein below. The transformation of compound (iii) to compound (x) illustrates methods of adding isoprene derivatives to a compound, which methods are suitable to make compound (iii) from compound (i).

The intermediates prepared above are converted to the compounds utilized in this invention as schematically illustrated below:

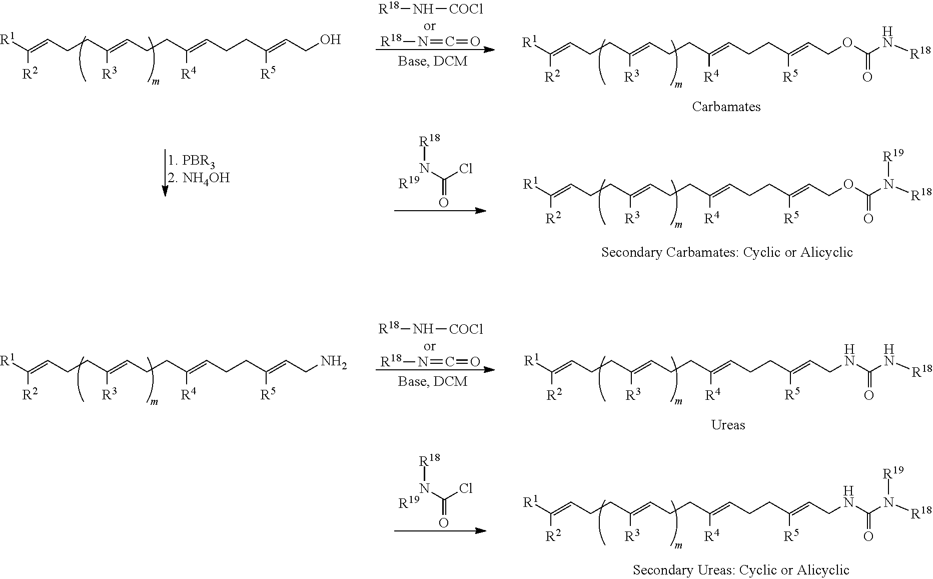

As used herein, for example, and without limitation, m is 0 or 1 and R¹-R⁵ are as defined herein, and are preferably alkyl, or more preferably methyl. Intermediate (ixa), prepared according to the scheme herein above, is converted to amino intermediate (ixb) via the corresponding bromide. Intermediates (ixa) and (ixb) are converted to the compounds utilized in this invention by reacting with suitable isocyanates or carbamoyl chlorides, which are prepared by art known methods. The thiocarbamates and thioureas of this invention are prepared according to the methods described above and replacing the isocyanates or the carbamoyl chlorides with isothiocyanates (R¹⁸—N═C═S) or thiocarbamoyl chlorides (R¹⁸—NH—C(═S)Cl or R¹⁸R¹⁹N—C(═S)Cl). These and other compounds utilized in this invention are also prepared by art known methods, which may require optional modifications as will be apparent to the skilled artisan upon reading this disclosure. Intermediates for synthesizing compounds utilized in this invention containing various R¹-R⁵ substituents are illustrated in the examples section and/or are well known to the skilled artisan.

Certain GGA derivatives utilized herein are synthesized as schematically shown below.

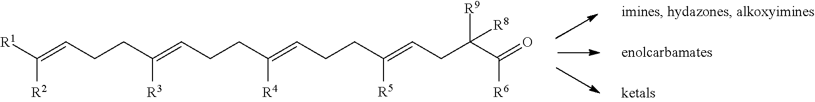

Certain compounds utilized herein are obtained by reacting compound (x) with the anion Q(−), which can be generated by reacting the compound QH with a base. Suitable nonlimiting examples of bases include hydroxide, hydride, amides, alkoxides, and the like. Various compounds utilized in this invention, wherein the carbonyl group is converted to an imine, a hydrazone, an alkoxyimine, an enolcarbamate, a ketal, and the like, are prepared following well known methods.

Other methods for making the compounds utilized in this invention are schematically illustrated below:

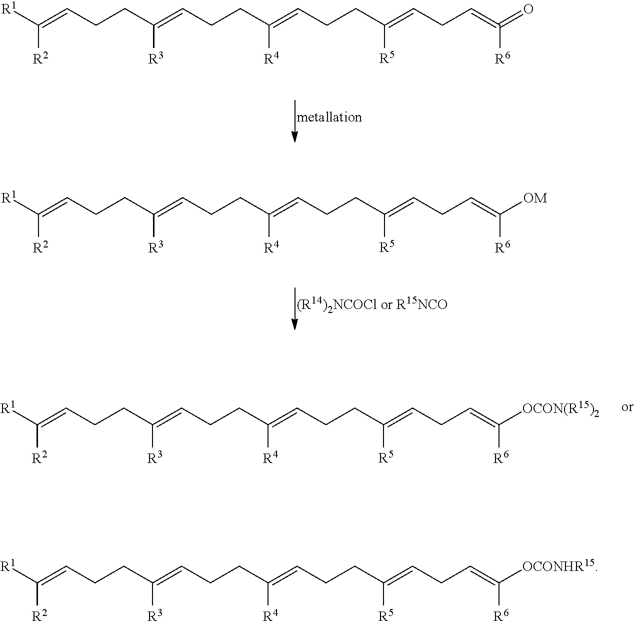

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine (R¹⁴)₂NH with phosgene or an equivalent reagent well known to the skilled artisan.

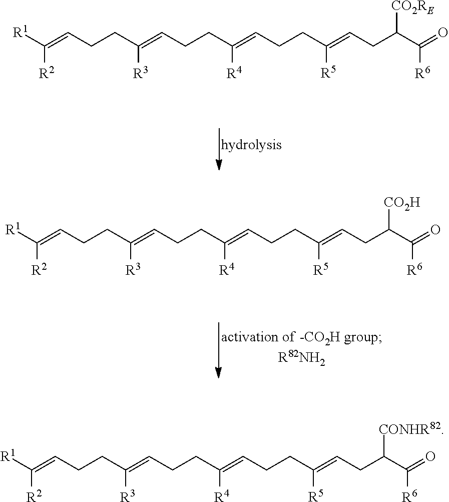

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine.

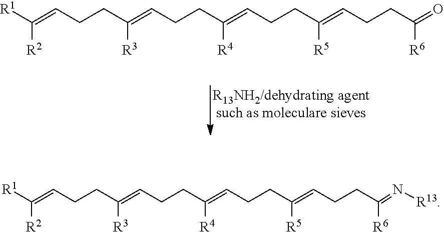

Various other compounds utilized in this invention are prepared from the compounds made in the scheme above based on art known methods.

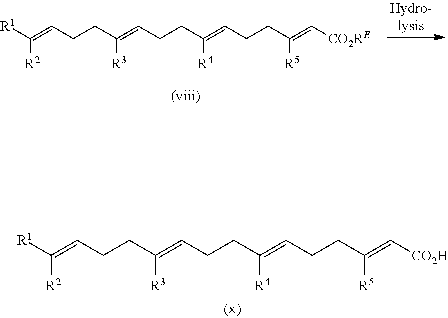

As shown above, R^{E} is alkyl.

The intermediates prepared above are converted to the compounds utilized in this invention as schematically illustrated below:

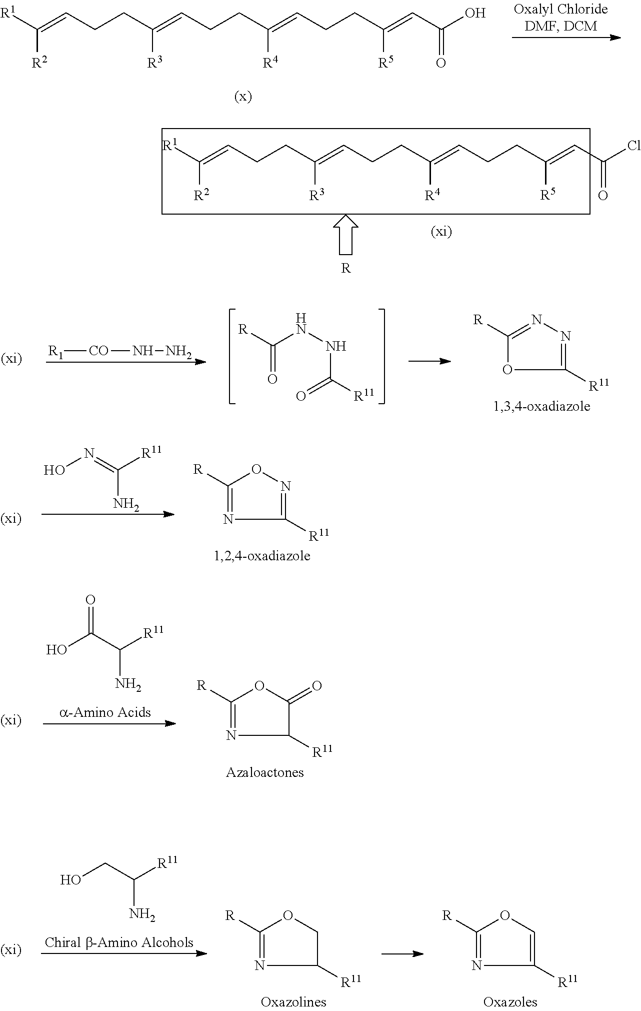

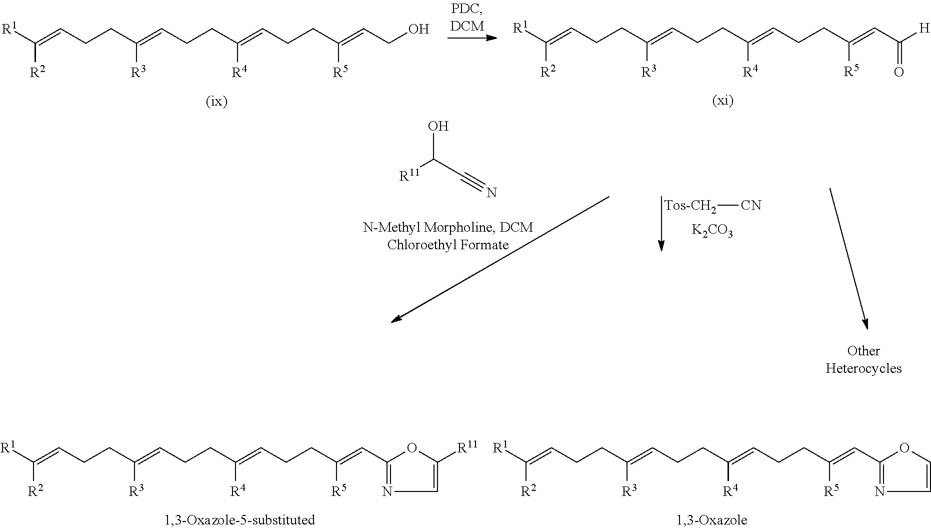

Compound (viii) is hydrolyzed to the carboxylic acid (x), which is then converted to the acid chloride (xi). Compound (xi) is reacted with a suitable nucleophile such as a hydrazide, a hydroxylamine, an amino alcohol, or an amino acid, and the intermediate dehydrated to provide a compound of Formula (I). Alternatively, the allylic alcohol (ix) is oxidized to the aldehyde (xi), which is then reacted with a cyanohydrin or cyanotosylmethane to provide further compounds utilized in this invention.

GGA derivatives utilized in this invention can also be synthesized employing art known methods and those disclosed here by alkene-aryl, alkene-heteroaryl, or alkene-akene couplings such as Heck, Stille, or Suzuki coupling. Such methods can use (vi) to prepare intermediate (xii) that can undergo Heck, Stille, or Suzuki coupling under conditions well known to the skilled artisan to provide compounds utilized in this invention.

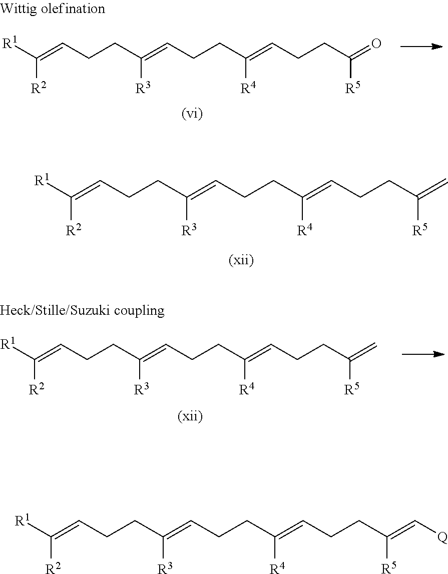

Higher and lower isoprenyl homologs of intermediates (x), (xi), and (xii), which are prepared following the methods disclosed here, can be similarly utilized to prepare other compounds utilized in this invention.

Compounds utilized in this invention are also prepared as shown below

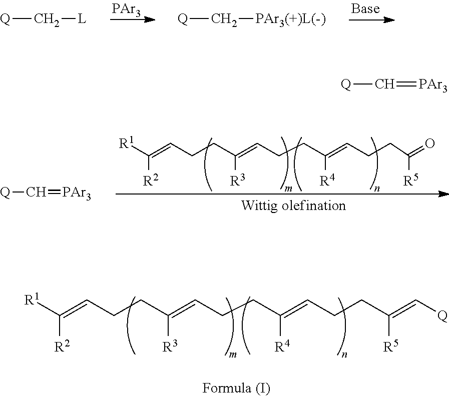

L and Q are as defined herein, Ar is a preferably an aryl group such as phenyl, the base employed is an alkoxide such as tertiarybutoxide, a hydride, or an alkyl lithium such as n-butyl lithium. Methods of carrying out the steps shown above are well known to the skilled artisan, as are conditions, reagents, solvents, and/or additives useful for performing the reactions and obtaining the compound of Formula (I) in the desired stereochemistry.

Other methods for making the compounds utilized in this invention are schematically illustrated below:

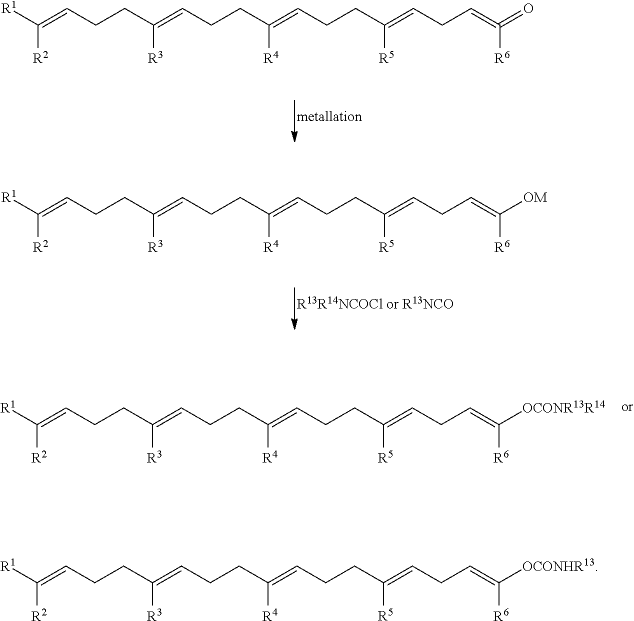

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine R¹³R¹⁴NH with phosgene or an equivalent reagent well known to the skilled artisan.

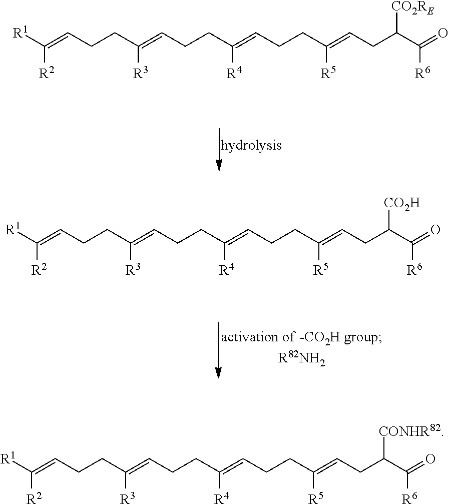

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine. Certain other methods of preparing the conjugates are shown below.

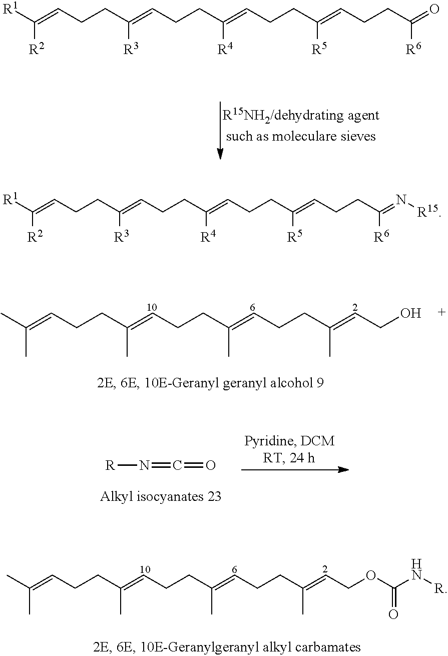

As shown above, R is a memantine or a riluzole residue.

### EXAMPLES

### Preparation of Co-Crystals of this Invention and Isolating GGA or a GGA Derivative with an Increased Trans Isomer Content

### Example 1

GGA or a derivative thereof (about 10 g) containing a mixture of cis and trans isomers (the starting mixture) is added to a 250 mL three necked round bottom flask containing isopropanol (about 75 mL). To this mixture is added thiourea (about 3 g), and optionally heated at about 90-95° C. for about 15 h. The solvent is distilled at reduced pressure at about 55-60° C. to provide the co-crystals of this invention.

If the GGA derivative is a solid, methanol (about 50 mL) is added to the co-crystals, stirred at 55-60° C. for about 20 min and gradually cooled to about 20-25° C. The solid formed is filtered, washed with methanol and dried under reduced pressure for about 3 h to obtain the GGA derivative having a higher percent of the trans isomer than in the starting mixture.

If the GGA derivative is a liquid, and for GGA, the co-crystals are decomposed with water and the GGA or the GGA derivative is extracted with light petroleum, or another suitable solvent as will be apparent to the skilled artisan.

### Example 2

GGA or a GGA derivative (a few mg) is dissolved in a little toluene (0.5-1.0 ml) and a saturated solution of thiourea in methanol (2-3 ml) is added. The co-crystals formed are collected after a few hours by centrifuging.

The co-crystals are washed with a little light petroleum, then decomposed with water and the GGA or the GGA derivative is extracted with light petroleum, or another suitable solvent as will be apparent to the skilled artisan.

### Example 3A

### Obtaining Trans and Cis GGA by Co-Crystallization

(5E,9E,13E/5Z,9E,13E)-geranylgeranylacetone (100 mg) containing 39% 5-cis and 61% all trans isomers is added to a small round bottom flak containing methanol (1.6 mL). To this mixture was added thiourea (300 mg), and heated until thiourea was dissolved. The mixture was cooled below 5° C. overnight. The solid formed is filtered (250.4 mg), dissolved in water (1 mL) and extracted with hexane (1 mL). The organic layer is separated and under reduced pressure removed to yield geranylgeranylacetone containing 22% cis and 78% as measured by GC. The supernatant from the filtration is extracted with hexane (1 mL). The organic solvent is removed under reduced pressure to yield geranylgeranylacetone containing 56% cis and 44% trans as measured by GC.

### Example 3B

### Obtaining Trans and Cis GGA by Co-Crystallization

(5E,9E,13E/5Z,9E,13E)-geranylgeranylacetone (100 mg) containing 76% 5-cis and 24% all trans isomers is added to a small round bottom flak containing methanol (0.9 mL). To this mixture was added thiourea (100 mg), and heated until thiourea was dissolved. The mixture was cooled below 5° C. overnight. The solid formed is filtered. The supernatant from the filtration is extracted with extracted with hexane (1 mL). The organic solvent is removed under reduced pressure to yield geranylgeranylacetone containing 86% cis and 14% trans as measured by GC.

### Example 4

### Obtaining Trans and Cis Ethyl Geranylgeranylate by Co-Crystallization

This example illustrates the usefulness of the invention provided herein for obtaining cis and trans forms of a compound having fewer chain carbon atoms than GGA. Ethyl (2E,6E,10E/2Z,6E,10E)-geranylgeranylate (100 mg; 106.2 μL) containing 22% cis and 77% trans isomers is added to a Craig tube containing methanol (1.63 mL). To this mixture was added thiourea (366 mg), and heated until thiourea was dissolved. The mixture was cooled below 5° C. with stirring for 18 h. The solid formed is filtered, dissolved in water (1 mL) and extracted with heptane (1 mL). The organic layer is separated and under reduced pressure removed to yield ethyl (2E,6E,10E/2Z,6E,10E)-geranylgeranylate containing 4% cis and 96% trans. The supernatant from the filtration is extracted with heptane (1 mL). The organic solvent is removed under reduced pressure to yield ethyl (2E,6E,10E/2Z,6E,10E)-geranylgeranylate containing 35% cis and 65% trans.

### Example 5A

### Obtaining Trans and Cis Geranylfarnesylacetone by Co-Crystallization

This example illustrates the usefulness of the invention provided herein for obtaining cis and trans forms of a compound having more chain carbon atoms than GGA. (5E,9E,13E,17E/5Z,9E,13E,17E/5E,9Z,13E,17E/5Z,9Z,13E,17E)-geranylfarnesylacetone (100 mg) containing 45% of a mixture of 5-cis-9-trans, 5-trans-9-cis and 5,9-di-cis isomers and 55% all-trans isomers is added to a Craig tube containing methanol (1.63 mL). To this mixture was added thiourea (366 mg), and heated until thiourea was dissolved. The mixture was cooled below 5° C. (−25° C.) with stirring for 18 h. The solid formed is filtered (312 mgs), dissolved in water (1 mL) and extracted with heptane (1 mL). The organic layer is separated and under reduced pressure removed to yield (5E,9E,13E,17E/5Z,9E,13E,17E/5E,9Z,13E,17E/5Z,9Z,13E,17E)-geranyl-farnesylacetone containing 77% of the all-trans isomer. The supernatant from the filtration is extracted with heptane (1 mL). The organic solvent is removed under reduced pressure to yield (5E,9E,13E,17E/5Z,9E,13E,17E/5E,9Z,13E,17E/5Z,9Z,13E,17E)-geranylfarnesylacetone containing 85% of a mixture of 5-cis-9-trans, 5-trans-9-cis and 5,9-di-cis isomers and 15% of the all-trans isomer.

### Example 5A

### Obtaining Trans and Cis Geranylfarnesylacetone by Co-Crystallization

(5E,9E,13E,17E/5Z,9E,13E,17E/5E,9Z,13E,17E/5Z,9Z,13E,17E)-geranylfarnesylacetone (100 mg) containing 29% of a mixture of 5-cis-9-trans, 5-trans-9-cis and 5,9-di-cis isomers and 71% all-trans isomers is added to a Craig tube containing methanol (1.63 mL). To this mixture was added thiourea (366 mg), and heated until thiourea was dissolved. The mixture was cooled in an ice bath at 0° C. with agitation for 0.5 h. The solid formed is filtered, dissolved in water (1 mL) and extracted with heptane (1 mL). The organic layer is separated and under reduced pressure removed to yield (5E,9E,13E,17E/5Z,9E,13E,17E/5E,9Z,13E,17E/5Z,9Z,13E,17E)-geranyl-farnesylacetone containing 87% of the all-trans isomer. The supernatant from the filtration is extracted with heptane (1 mL). The organic solvent is removed under reduced pressure to yield (5E,9E,13E,17E/5Z,9E,13E,17E/5E,9Z,13E,17E/5Z,9Z,13E,17E)-geranylfarnesylacetone containing 56.5% of a mixture of 5-cis-9-trans, 5-trans-9-cis and 5,9-di-cis isomers and 43.5% of the all-trans isomer.

The examples demonstrate that GGA and GGA derivatives having different functional groups and chain carbon numbers are efficiently enriched into trans and cis forms in accordance to this invention. Furthermore, multiple rounds of crystallization provides enhanced amount of the trans and cis isomers in a cis-trans mixture. Based on the illustrative examples provided herein and based on methods known for other compounds, a skilled artisan can adapt and/or apply such methods for obtaining trans and.or cis forms of GGA and GGA derivatives.

1. A co-crystal or co-precipitate comprising GGA, geranylgeranyl alcohol, or another GGA derivative, and urea and/or thiourea, wherein the GGA, geranylgeranyl alcohol, or the another GGA derivative exists at least 80%, or at least 90%, or at least 95%, or at least 99% in the trans isomer.

2. The co-crystal or co-precipitate of claim 1 admixed with a composition comprising GGA, geranylgeranyl alcohol, or the other GGA derivative, wherein the GGA, geranylgeranyl alcohol, or the another GGA derivative in the composition exists substantially less in the trans form compared to the GGA, geranylgeranyl alcohol, or the another GGA derivative that is complexed as part of the co-crystal or the co-precipitate.

3. The co-crystal or co-precipitate of claim 1, which is crystalline.

4. A crystalline GGA derivative existing in the trans form or substantially in the trans form.

5. A process of preparing a co-crystal or a co-precipitate of GGA, geranylgeranyl alcohol, or another GGA derivative, and urea and/or thiourea, wherein the GGA, geranylgeranyl alcohol, or the another GGA derivative exists at least 80%, or at least 90%, or at least 95%, or at least 99% as the trans isomer, the process comprising contacting a mixture of cis and trans isomers of GGA, geranylgeranyl alcohol, or the another GGA derivative with a composition comprising urea and/or thiourea under conditions sufficient to form the co-crystal or the co-precipitate, to provide the co-crystal or the co-precipitate.

6. The process of claim 5, further comprising isolating the GGA, geranylgeranyl alcohol, or the other GGA derivative from the co-crystal, to provide the GGA, geranylgeranyl alcohol, or the another GGA derivative having at least 80%, or at least 90%, or at least 95%, or at least 99% of the trans isomer.

7. A process of separating GGA, geranylgeranyl alcohol, or another GGA derivative existing in the trans form or substantially in the trans form, the process comprising contacting a co-crystal or co-precipitate comprising GGA, geranylgeranyl alcohol, or the another GGA derivative, and urea and/or thiourea, wherein the GGA, geranylgeranyl alcohol, or the another GGA derivative complexed in the co-crystal or the co-precipitate exists in the trans form or substantially in the trans form, with a solvent that selectively dissolves either the GGA, geranylgeranyl alcohol, or the another GGA derivative, or the urea and/or the thiourea, under conditions sufficient for such dissolution.

8. A process for preparing a compound of formula (XXXI): 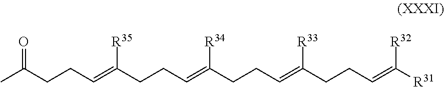
said process comprising:
hydrolyzing a compound of formula (XXXII): 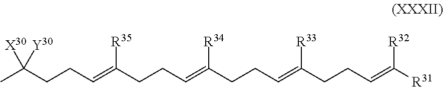
wherein:
X³⁰ and Y³⁰ are each independently OR³⁶, SR³⁶, or X³⁰ and Y³⁰ together with the carbon atom they are attached to form a ring of formula: 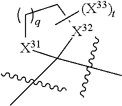
wherein each R³⁶ is independently C₁-C₆ alkyl,
each X³¹ and X³² are independently O, or S; q is 1 or 2; each X³³ is independently C₁-C₆ alkyl; t is 0, 1, 2, or 3, and
each of R³¹, R³², R³³, R³⁴, and R³⁵ is independently H or C₁-C₆ alkyl or R¹ and R² together with the carbon atom they are joined to form a C₅-C₆ cycloalkyl optionally substituted with 1-3 C₁-C₆ alkyl.

9. The process of claim 8, wherein the compound of formula (XXXII): 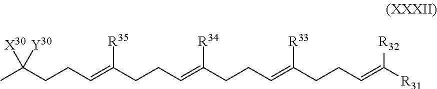
is prepared comprising:
contacting a compound of formula (XXXIII): 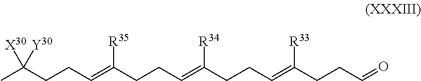
with a reagent of formula: 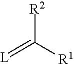
wherein L³⁰ is P(R^{z})₃, P(O)(R^{z})₂, SO₂R^{z}, or Si(R^{z})₃; and wherein R^{z} is a C₁-C₆ alkyl group or an aryl group;
under conditions suitable for olefination of compound of formula (XXXIII) to produce a compound of formula (XXXII).

10. A process for preparing a compound of formula (YXI): 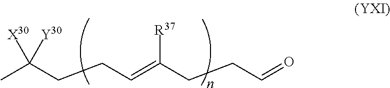
said process comprising:
oxidizing a compound of formula (YXII): 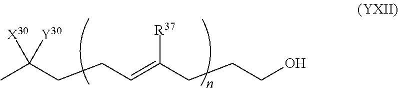
wherein:
X and Y are each independently OR³⁶, SR³⁶, or X³⁰ and Y³⁰ together with the carbon atom they are attached to form a ring of formula: 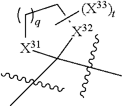
wherein each R³⁶ is independently C₁-C₆ alkyl,
each X³¹ and X³² are independently O, or S; q is 1 or 2; each X³ is independently C₁-C₆ alkyl; t is 0, 1, 2, or 3,
each of R³⁷ independently is H or C₁-C₆ alkyl; and
n is 1-5,
under suitable conditions to provide a compound of formula (YXI).

11. The process of claim 10 wherein the compound of formula (YXII) 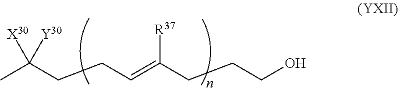
is prepared comprising:
reducing a compound of formula (YXIII) 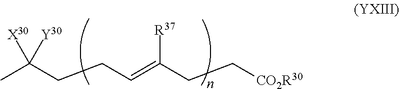
under conditions suitable to provide a compound of formula (YXII).

12. The process of claim 11, wherein the compound of formula (YXIII): 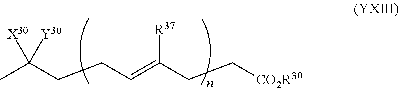
is prepared comprising:
contacting an orthoacetate of formula CH₃C(OR³⁰)₃, wherein R³⁰ is C₁-C₆ alkyl, with a compound of formula (YXIV): 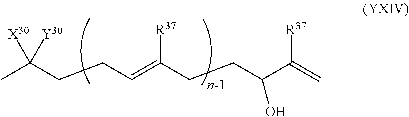
wherein X, Y, and R⁷ are defined as in formula (XXXII).

13. The process of claim 12, wherein the compound of formula (YXIV): 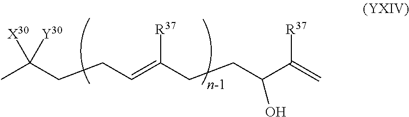
is prepared comprising:
contacting of a compound of formula (YXV): 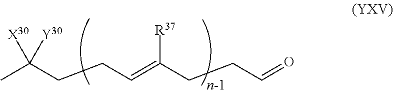
with an anion of formula R³⁷C(—)═CH₂.

14. The process of claim 8 or 9, wherein R³¹-R³⁵ are methyl.

15. The process of any one of claims 10-13, wherein R³⁷ is methyl.

16. A co-crystal or co-precipitate comprising a compound selected from the group consisting of: 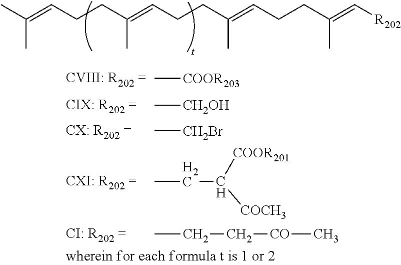
and urea and/or thiourea, wherein the compound exists at least 80%, or at least 90%, or at least 95%, or at least 99% in the trans isomer.

17. A process of preparing GGA, geranylgeranyl alcohol, or another GGA derivative, wherein the GGA, geranylgeranyl alcohol, or the another GGA derivative exists at least 50%, at least 80%, or at least 90%, or at least 95%, or at least 99% as the cis isomer, the process comprising:
contacting a mixture of cis and trans isomers of GGA, geranylgeranyl alcohol, or the another GGA derivative with a composition comprising urea and/or thiourea under conditions sufficient to form the co-crystal or the co-precipitate,
separating the co-crystal or the co-precipitate to obtain a mother liquor comprising the GGA, geranylgeranyl alcohol, or the another GGA derivative existing at least 50%, at least 80%, or at least 90%, or at least 95%, or at least 99% as the cis isomer.

18. The process of claim 17, further comprising isolating the GGA, geranylgeranyl alcohol, or the another GGA derivative existing at least 50%, at least 80%, or at least 90%, or at least 95%, or at least 99% as the cis isomer.

19. The process of claim 17 or 18, wherein the GGA derivative is selected from the group consisting of:

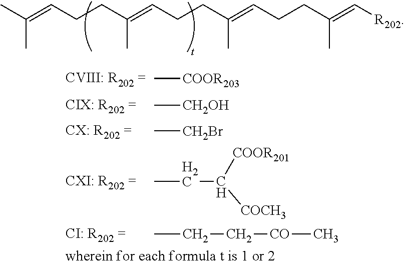

20. The process of claim 17 or 18, further comprising:
contacting the GGA, geranylgeranyl alcohol, or the another GGA derivative existing at least 50%, at least 80%, or at least 90%, or at least 95%, or at least 99% as the cis isomer with a composition comprising urea and/or thiourea under conditions sufficient to form a second co-crystal or a second co-precipitate, and
separating the second co-crystal or the second co-precipitate to obtain a second mother liquor comprising the GGA, geranylgeranyl alcohol, or the another GGA derivative existing at least 50%, at least 80%, or at least 90%, or at least 95%, or at least 99% as the cis isomer.

21. The process of any one of claim 17-20, wherein the GGA, geranylgeranyl alcohol, or the another GGA derivative exists at less than 50% as the cis isomer.
